SEQUENCE LISTING (291 sequences)

SEQ ID NO: 1 - protein, 6 aa, synthetic construct
SGYYWN

SEQ ID NO: 2 - protein, 16 aa, synthetic construct
YISYSGSNNYNPSLKN

SEQ ID NO: 3 - protein, 7 aa, synthetic construct
GGYYFDY

SEQ ID NO: 4 - protein, 17 aa, synthetic construct
KSSQSLLNTNNQKNYLA

SEQ ID NO: 5 - protein, 7 aa, synthetic construct
FASTRES

SEQ ID NO: 6 - protein, 9 aa, synthetic construct
QQHYNAPRT

SEQ ID NO: 7 - protein, 116 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSS

SEQ ID NO: 8 - protein, 113 aa, synthetic construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIK

SEQ ID NO: 9 - protein, 446 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 220 aa, synthetic construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, synthetic construct
IYGMS

SEQ ID NO: 13 - protein, 17 aa, synthetic construct
TISRDASYTYFPDSVKG

SEQ ID NO: 14 - protein, 12 aa, synthetic construct
LGDNDRGYAMDY

SEQ ID NO: 15 - protein, 16 aa, synthetic construct
RSSQSVVHVNANTYLE

SEQ ID NO: 16 - protein, 7 aa, synthetic construct
KVSNRFP

SEQ ID NO: 17 - protein, 9 aa, synthetic construct
FQGSHVPRT

SEQ ID NO: 18 - protein, 121 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSS

SEQ ID NO: 19 - protein, 112 aa, synthetic construct
DVVMTQSPLSLPVTLGQPASISCRSSQSVVHVNANTYLEWYQQRPGQSPRLLIYKVSNRFPGVPDRFSGSGSGTDFTLKISRVEAEDVGVYYCFQGSHVPRTFGGGTKVEIK

SEQ ID NO: 20 - protein, 451 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 451 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 219 aa, synthetic construct
DVVMTQSPLSLPVTLGQPASISCRSSQSVVHVNANTYLEWYQQRPGQSPRLLIYKVSNRFPGVPDRFSGSGSGTDFTLKISRVEAEDVGVYYCFQGSHVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 15 aa, synthetic construct
GGGGSGGGGSGGGGS

SEQ ID NO: 24 - protein, 5 aa, synthetic construct
SNCMG

SEQ ID NO: 25 - protein, 17 aa, synthetic construct
VICTGGGSPSYADSVKG

SEQ ID NO: 26 - protein, 16 aa, synthetic construct
DLLRAGTPLSSYEFNY

SEQ ID NO: 27 - protein, 125 aa, synthetic construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 28 - protein, 586 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 29 - protein, 220 aa, synthetic construct; identical to SEQ ID NO: 11

SEQ ID NO: 30 - protein, 586 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 31 - protein, 220 aa, synthetic construct; identical to SEQ ID NO: 11

SEQ ID NO: 32 - protein, 586 aa, synthetic construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSSGGGGSGGGGSGGGGSQVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 220 aa, synthetic construct; identical to SEQ ID NO: 11

SEQ ID NO: 34 - protein, 446 aa, synthetic construct; identical to SEQ ID NO: 9

SEQ ID NO: 35 - protein, 360 aa, synthetic construct
QVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSSGGGGSGGGGSGGGGSDIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 591 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 37 - protein, 219 aa, synthetic construct; identical to SEQ ID NO: 22

SEQ ID NO: 38 - protein, 591 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 39 - protein, 219 aa, synthetic construct; identical to SEQ ID NO: 22

SEQ ID NO: 40 - protein, 220 aa, synthetic construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 41 - protein, 232 aa, synthetic construct
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 42 - protein, 445 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 445 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 585 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 45 - protein, 585 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 46 - protein, 220 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 220 aa, synthetic construct
CPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 220 aa, synthetic construct
CPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 49 - protein, 220 aa, synthetic construct
CPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 50 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 51 - protein, 220 aa, synthetic construct
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 220 aa, synthetic construct
CPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 220 aa, synthetic construct
CPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 220 aa, synthetic construct
CPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 220 aa, synthetic construct
CPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 220 aa, synthetic construct
CPPCPAPDLLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 5 aa, synthetic construct
SYAIS

SEQ ID NO: 58 - protein, 17 aa, synthetic construct
GIIPIFGTANYAQKFQG

SEQ ID NO: 59 - protein, 22 aa, synthetic construct
DSRKYYYDSSGPALWGFDAFDI

SEQ ID NO: 60 - protein, 11 aa, synthetic construct
RASQSISSYLN

SEQ ID NO: 61 - protein, 7 aa, synthetic construct
AASSLQS

SEQ ID NO: 62 - protein, 9 aa, synthetic construct
QQSYSTLST

SEQ ID NO: 63 - protein, 131 aa, synthetic construct
EVQLLESGAEVKKPGSSVKVSCKASGGTFSSYAISWVRQAPGQGLEWMGGIIPIFGTANYAQKFQGRVTITADESTSTAYMELSSLRSEDTAVYYCARDSRKYYYDSSGPALWGFDAFDIWGQGTMVTVSS

SEQ ID NO: 64 - protein, 107 aa, synthetic construct
NIQLTQSPSSLSASVGDRVTITCRASQSISSYLNWYQQKPGKAPKLLIYAASSLQSGVPSRFSGSGSGTDFTLTISSLQPEDFATYYCQQSYSTLSTFGPGTKVDIK

SEQ ID NO: 65 - protein, 590 aa, synthetic construct
QVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVTAGMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 66 - protein, 219 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCLQATYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 21 aa, synthetic construct
EPPLSNYGDYATEQYYYGMDV

SEQ ID NO: 68 - protein, 590 aa, synthetic construct
QVQLVQSGAEVKKPGASVKVSCKASGYTFTNYVMHWVRQAPGQGLEWMGYINPYSGGIRYNEKFKGRVTMTRDTSTSTVYMELSSLRSEDTAVYYCARRERGVYYGMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 69 - protein, 219 aa, synthetic construct; identical to SEQ ID NO: 66

SEQ ID NO: 70 - protein, 9 aa, synthetic construct
QQSYSTPLT

SEQ ID NO: 71 - protein, 130 aa, synthetic construct
QVQLMQSGAEVKKPGSSVKVSCKASGGTFSSYAISWVRQAPGQGLEWMGGIIPIFGTANYAQKFQGRVTITADESTSTAYMELSSLRSEDTAVYYCAREPPLSNYGDYATEQYYYGMDVWGQGTLVTVSS

SEQ ID NO: 72 - protein, 107 aa, synthetic construct
NIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 73 - protein, 593 aa, synthetic construct
EVQLVESGGGLVKPGGSLRLSCAASGFTFSRYAMSWVRQAPGKRLEWVSSISSGGNTYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 74 - protein, 621 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCYQATYFPYTFGQGTKLEIKGGGGSGGGGSGGGGSQVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMSEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 75 - protein, 20 aa, synthetic construct
APMVRGVPPTPYYYYYGMDV

SEQ ID NO: 76 - protein, 11 aa, synthetic construct
RASQSVSNYLA

SEQ ID NO: 77 - protein, 7 aa, synthetic construct
DASNRAT

SEQ ID NO: 78 - protein, 8 aa, synthetic construct
QQRSNWPS

SEQ ID NO: 79 - protein, 129 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAPMVRGVPPTPYYYYYGMDVWGQGTTVTVSS

SEQ ID NO: 80 - protein, 106 aa, synthetic construct
EIVLTQSPATLSLSPGERATLSCRASQSVSNYLAWYQQKPGQAPRLLIYDASNRATGIPARFSGSGSGTDFTLTISSLEPEDFAVYYCQQRSNWPSFGQGTKVEIK

SEQ ID NO: 81 - protein, 214 aa, synthetic construct
DIQMTQSPSSLSASVGDRVTITCRASQDIYSYLSWFQQKPGKAPKTLIYRENRLVDAVPSRFSGSGSGQDYTLTISSLQPEDFATYYCLQYDEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 593 aa, synthetic construct
EVQLVESGGGLVKPGGSLRLSCAASGFTFRSYAMSWVRQAPGKRLEWVSSISSGGTRYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 83 - protein, 18 aa, synthetic construct
TPELLWFGELGGAYYFDY

SEQ ID NO: 84 - protein, 11 aa, synthetic construct
RASESISSWLA

SEQ ID NO: 85 - protein, 7 aa, synthetic construct
KASTLEN

SEQ ID NO: 86 - protein, 10 aa, synthetic construct
QQYRSHWSST

SEQ ID NO: 87 - protein, 127 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASTPELLWFGELGGAYYFDYWGQGTLVTVSS

SEQ ID NO: 88 - protein, 108 aa, synthetic construct
NIQLTQSPSTLSASVGDRVTITCRASESISSWLAWYQQKPGEAPKLLIYKASTLENGVPSRFSGSGSETEFTLTISSLQPDDFATYYCQQYRSHWSSTFGPGTKVDIK

SEQ ID NO: 89 - protein, 621 aa, synthetic construct; identical to SEQ ID NO: 74

SEQ ID NO: 90 - protein, 214 aa, synthetic construct; identical to SEQ ID NO: 81

SEQ ID NO: 91 - protein, 14 aa, synthetic construct
ANFNIYYYYYGMDV

SEQ ID NO: 92 - protein, 16 aa, synthetic construct
RSSQSLLHSNGYNYLD

SEQ ID NO: 93 - protein, 7 aa, synthetic construct
LGSNRAS

SEQ ID NO: 94 - protein, 9 aa, synthetic construct
MQGTHWPRT

SEQ ID NO: 95 - protein, 123 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARANFNIYYYYYGMDVWGQGTLVTVSS

SEQ ID NO: 96 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGIYYCMQGTHWPRTFGQGTKVEIK

SEQ ID NO: 97 - protein, 5 aa, synthetic construct
SYEMN

SEQ ID NO: 98 - protein, 17 aa, synthetic construct
RIKSKTDGGTTDYAAPV

SEQ ID NO: 99 - protein, 11 aa, synthetic construct
DLAAVAGLFDY

SEQ ID NO: 100 - protein, 11 aa, synthetic construct
QASQDISNYLN

SEQ ID NO: 101 - protein, 7 aa, synthetic construct
DASNLET

SEQ ID NO: 102 - protein, 9 aa, synthetic construct
QQSYSTPWK

SEQ ID NO: 103 - protein, 122 aa, synthetic construct
EVQLLESGGGLVQPGGSLRLSCAASGFTFSSYEMNWVRQAPGKGLEWVGRIKSKTDGGTTDYAAPVKGRFTISRDDSKNTLYLQMNSLKTEDTAVYYCTTDLAAVAGLFDYWGQGTTVTVSS

SEQ ID NO: 104 - protein, 107 aa, synthetic construct
DIQLTQSPSSLSASVGDRVTITCQASQDISNYLNWYQQKPGKAPKLLIYDASNLETGVPSRFSGSGSGTDFTLTISSLQPEDFATYYCQQSYSTPWKFGQGTKVEIK

SEQ ID NO: 105 - protein, 593 aa, synthetic construct; identical to SEQ ID NO: 73

SEQ ID NO: 106 - protein, 17 aa, synthetic construct
RIIPIFGIANYAQKFQG

SEQ ID NO: 107 - protein, 17 aa, synthetic construct
TGDYDILTGSYYYGMDV

SEQ ID NO: 108 - protein, 11 aa, synthetic construct
RASQGIRNDLG

SEQ ID NO: 109 - protein, 621 aa, synthetic construct
DIVMTQTPLSLSVTPGQPASISCKSSQSLLHSNGKTYLNWYLQKPGQSPQLLIYLVSKLSSGVPDRFSGSGSGTDFTLKISRVEAEDVGVYYCLQATYFPYTFGQGTKLEIKGGGGSGGGGSGGGGSQVQLVQSGAEVKKPGASVKVSCKASGYRFTTYVMHWVRQAPGQGLEWMGYINPYNGVIRYNEKFKGRVTMTRDTSTSTVYMELSSLRSEDTAVYYCARRERGVYYGMDEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 110 - protein, 593 aa, synthetic construct
EVQLVESGGGLVKPGGSLRLSCAASGFTFSRYAMSWVRQAPGKRLEWVSSISSGGNTYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 111 - protein, 214 aa, synthetic construct; identical to SEQ ID NO: 81

SEQ ID NO: 112 - protein, 593 aa, synthetic construct
EVQLVESGGGLVKPGGSLRLSCAASGFTFRSYAMSWVRQAPGKRLEWVSSISSGGTRYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 113 - protein, 214 aa, synthetic construct; identical to SEQ ID NO: 81

SEQ ID NO: 114 - protein, 590 aa, synthetic construct
QVQLVQSGAEVKKPGASVKVSCKASGYRFTTYVMHWVRQAPGQGLEWMGYINPYNGVIRYNEKFKGRVTMTRDTSTSTVYMELSSLRSEDTAVYYCARRERGVYYGMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 115 - protein, 219 aa, synthetic construct
DIVMTQTPLSLSVTPGQPASISCKSSQSLLHSNGKTYLNWYLQKPGQSPQLLIYLVSKLSSGVPDRFSGSGSGTDFTLKISRVEAEDVGVYYCLQATYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 590 aa, synthetic construct
QVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMSEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 117 - protein, 219 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCYQATYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 590 aa, synthetic construct
QVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVYYGMSEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 119 - protein, 219 aa, synthetic construct
DIVMTQTPLSLSVTPGQPASISCKSSQSLLHSNDKTYLNWYLQKPGQSPQLLIYLVSKLESGVPDRFSGSGSGTDFTLKISRVEAEDVGVYYCYQATYFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 9 aa, synthetic construct
LQDYNYPFT

SEQ ID NO: 121 - protein, 126 aa, synthetic construct
QVQLVQSGAEVKKPGSSVKVSCKASGGTFSSYAISWVRQAPGQGLEWMGRIIPIFGIANYAQKFQGRVTITADKSTSTAYMELSSLRSEDTAVYYCAITGDYDILTGSYYYGMDVWGQGTTVTVSS

SEQ ID NO: 122 - protein, 107 aa, synthetic construct
NIQLTQSPSSLSASVGDRVTITCRASQGIRNDLGWYQQKPGKAPKLLIYAASSLQSGVPSRFSGSGSGTDFTLTISSLQPEDFATYYCLQDYNYPFTFGPGTKVDIK

SEQ ID NO: 123 - protein, 5 aa, synthetic construct
DYYLS

SEQ ID NO: 124 - protein, 214 aa, synthetic construct; identical to SEQ ID NO: 81

SEQ ID NO: 125 - protein, 22 aa, synthetic construct
GGPHYDFWSGYTPGQHGGAFDI

SEQ ID NO: 126 - protein, 12 aa, synthetic construct
RASQSVSSSYLA

SEQ ID NO: 127 - protein, 7 aa, synthetic construct
GASSRAT

SEQ ID NO: 128 - protein, 9 aa, synthetic construct
QQRSNWRNT

SEQ ID NO: 129 - protein, 131 aa, synthetic construct
EVQLLESGGGLVTPGGSLRLSCTASGFTFSDYYLSWVRQAPGKGLEWMGGIIPIFGTANYAQKFQGRVTITADESTSTAYMELSSLRSEDTAVYYCARGGPHYDFWSGYTPGQHGGAFDIWGQGTMVTVSS

SEQ ID NO: 130 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQRSNWRNTFGQGTRLEIK

SEQ ID NO: 131 - protein, 593 aa, synthetic construct
EVQLVESGGGLVKPGGSLRLSCAASGFTFRSYAMSWVRQAPGKRLEWVSSISSGGTRYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 132 - protein, 621 aa, synthetic construct
DIVMTQTPLSLSVTPGQPASISCKSSQSLLHSNDKTYLNWYLQKPGQSPQLLIYLVSKLESGVPDRFSGSGSGTDFTLKISRVEAEDVGVYYCYQATYFPYTFGQGTKLEIKGGGGSGGGGSGGGGSQVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVYYGMSEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 133 - protein, 19 aa, synthetic construct
DSGSSITMVRGGDYYYMDV

SEQ ID NO: 134 - protein, 11 aa, synthetic construct
RASQSVSSYLA

SEQ ID NO: 135 - protein, 214 aa, synthetic construct; identical to SEQ ID NO: 81

SEQ ID NO: 136 - protein, 9 aa, synthetic construct
QQRSNWPPT

SEQ ID NO: 137 - protein, 128 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSGSSITMVRGGDYYYMDVWGKGTTVTVSS

SEQ ID NO: 138 - protein, 107 aa, synthetic construct [sequence omitted]

SEQ ID NO: 139 - protein, 5 aa, synthetic construct
YHAIS

SEQ ID NO: 140 - protein, 17 aa, synthetic construct
GIIPILGTANYAQKFQG

SEQ ID NO: 141 - protein, 16 aa, synthetic construct
GTTAARYYYYYYYMDV

SEQ ID NO: 142 - protein, 593 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGGSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 143 - protein, 621 aa, synthetic construct
DIVMTQTPLSLSVTPGQPASISCKSSQSLLHSNGKTYLNWYLQKPGQSPQLLIYLVSKLSSGVPDRFSGSGSGTDFTLKISRVEAEDVGVYYCLQATYFPYTFGQGTKLEIKGGGGSGGGGSGGGGSQVQLVQSGAEVKKPGASVKVSCKASGYRFTTYVMHWVRQAPGQGLEWMGYINPYNGVIRYNEKFKGRVTMTRDTSTSTVYMELSSLRSEDTAVYYCARRERGVYYGMDEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 144 - protein, 9 aa, synthetic construct
QQYDNLPLT

SEQ ID NO: 145 - protein, 126 aa, synthetic construct
QVQLVQSGAEVKKPGSSVKVSCKASGGTFSRYHAISWVRQAPGQGLEWMGGIIPILGTANYAQKFQGRVTITADESTSTAYMELSSLRSEDTAVYYCASGTTAARYYYYYYYMDVWGKGTTVTVSS

SEQ ID NO: 146 - protein, 107 aa, synthetic construct
NIQMTQSPSSLSASVGDRVTITCQASQDISNYLNWFHQKPGKAPNLLIYDASNLETGVPSRFSGSGSGTDFTFTISSLQPEDIGTYYCQQYDNLPLTFGGGTKVDIK

SEQ ID NO: 147 - protein, 214 aa, synthetic construct
DIQMTQSPSSLSASVGDRVTITCRASQDIYSYLSWFQQKPGKAPKTLIYRANRLVDAVPSRFSGSGSGQDYTLTISSLQPEDFATYYCLQYDEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 593 aa, synthetic construct; identical to SEQ ID NO: 142

SEQ ID NO: 149 - protein, 27 aa, synthetic construct
SITDYYDSSGYYFRPHFNTGYYYGMDV

SEQ ID NO: 150 - protein, 11 aa, synthetic construct
RASQGINNYLA

SEQ ID NO: 151 - protein, 7 aa, synthetic construct
AASTLQS

SEQ ID NO: 152 - protein, 9 aa, synthetic construct
QQYDTFSET

SEQ ID NO: 153 - protein, 136 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARSITDYYDSSGYYFRPHFNTGYYYGMDVWGQGTTVTVSS

SEQ ID NO: 154 - protein, 107 aa, synthetic construct
NIQLTQSPSSLSASVGDRVTITCRASQGINNYLAWYQQKPGKVPKLLIYAASTLQSGVPSRFSGSGSGTDFTLTITNLQPDDFATYYCQQYDTFSETFGPGTKVEIK

SEQ ID NO: 155 - protein, 214 aa, synthetic construct; identical to SEQ ID NO: 147

SEQ ID NO: 156 - protein, 593 aa, synthetic construct; identical to SEQ ID NO: 142

SEQ ID NO: 157 - protein, 621 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCLQATYFPYTFGQGTKLEIKGGGGSGGGGSGGGGSQVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVTAGMDEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 158 - protein, 11 aa, synthetic construct
RASQSISGWLA

SEQ ID NO: 159 - protein, 7 aa, synthetic construct
RTSYLES

SEQ ID NO: 160 - protein, 9 aa, synthetic construct
QHYDTFSRA

SEQ ID NO: 161 - protein, 131 aa, synthetic construct
EVQLLESGAEVKKPGSSVKVSCKASGGTFSSYAISWVRQAPGQGLEWMGGIIPIFGTANYAQKFQGRVTITADESTSTAYMELSSLRSEDTAVYYCARGGPHYDFWSGYTPGQHGGAFDIWGQGTMVTVSS

SEQ ID NO: 162 - protein, 107 aa, synthetic construct
NIQMTQSPSTLSASLGDRVSITCRASQSISGWLAWYQQKPGKAPKLLIYRTSYLESGVPSRFSAGGSGTEFTLTISSLQPDDFATYYCQHYDTFSRAFGQGTKVDIK

SEQ ID NO: 163 - protein, 214 aa, synthetic construct; identical to SEQ ID NO: 147

SEQ ID NO: 164 - protein, 593 aa, synthetic construct; identical to SEQ ID NO: 142

SEQ ID NO: 165 - protein, 20 aa, synthetic construct
EGPDYGDYSWSMDYYYGMDV

SEQ ID NO: 166 - protein, 12 aa, synthetic construct
RASQSVNSRYLA

SEQ ID NO: 167 - protein, 7 aa, synthetic construct
GASTRAT

SEQ ID NO: 168 - protein, 9 aa, synthetic construct
QQYGTFSIT

SEQ ID NO: 169 - protein, 129 aa, synthetic construct
EVQLVQSGAEVKKPGASVKVSCKASGGTFSSYAISWVRQAPGQGLEWMGGIIPIFGTANYAQKFQGRVTITADESTSTAYMELSSLRSEDTAVYYCAREGPDYGDYSWSMDYYYGMDVWGQGTMVTVSS

SEQ ID NO: 170 - protein, 108 aa, synthetic construct
EIVMTQSPGTLSLSPGERATLSCRASQSVNSRYLAWYQHKPGQAPRLLMYGASTRATGIPDRFSGSGSGTDFTLTISRLEPEDFAVYYCQQYGTFSITFGQGTRLEIK

SEQ ID NO: 171 - protein, 25 aa, synthetic construct
GGGGSGGGGSGGGGSGGGGSGGGGS

SEQ ID NO: 172 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 107 aa, synthetic construct [sequence omitted]

SEQ ID NO: 174 - protein, 611 aa, synthetic construct
EVQLLESGGGLVTPGGSLRLSCTASGFTFSDYYLSWVRQAPGKGLEWMGGIIPIFGTANYAQKFQGRVTITADESTSTAYMELSSLRSEDTAVYYCARGGPHYDFWSGYTPGQHGGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 175 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQRSNWRNTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 611 aa, synthetic construct
EVQLLESGAEVKKPGSSVKVSCKASGGTFSSYAISWVRQAPGQGLEWMGGIIPIFGTANYAQKFQGRVTITADESTSTAYMELSSLRSEDTAVYYCARGGPHYDFWSGYTPGQHGGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 177 - protein, 214 aa, synthetic construct
NIQMTQSPSTLSASLGDRVSITCRASQSISGWLAWYQQKPGKAPKLLIYRTSYLESGVPSRFSAGGSGTEFTLTISSLQPDDFATYYCQHYDTFSRAFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 480 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 179 - protein, 214 aa, synthetic construct
NIQLTQSPSSLSASVGDRVTITCRASQSISSYLNWYQQKPGKAPKLLIYAASSLQSGVPSRFSGSGSGTDFTLTISSLQPEDFATYYCQQSYSTLSTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 606 aa, synthetic construct
QVQLVQSGAEVKKPGSSVKVSCKASGGTFSRYHAISWVRQAPGQGLEWMGGIIPILGTANYAQKFQGRVTITADESTSTAYMELSSLRSEDTAVYYCASGTTAARYYYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 181 - protein, 214 aa, synthetic construct
NIQMTQSPSSLSASVGDRVTITCQASQDISNYLNWFHQKPGKAPNLLIYDASNLETGVPSRFSGSGSGTDFTFTISSLQPEDIGTYYCQQYDNLPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 447 aa, synthetic construct
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 215 aa, synthetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 459 aa, synthetic construct
EVQLVESGGGLEQPGRSLRLSCTASGFAFGDHTMSWVRQAPGKGLEWVGFIRSRAYGGTTEYAASVKGRFTISRDDSKSIAYLQMDSLKTEDTAVYYCTSGGYDSSLHYYYYYHGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 446 aa, synthetic construct
EVQLVESGGGLVQPGGSLRLSCAASGYSITNDYAWNWVRQAPGKGLEWVGYISYSGYTTYNPSIKSRFTISRDTSKNTLYLQMNSLRAEDTAVYYCARWTSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 214 aa, synthetic construct
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 123 aa, synthetic construct
EVKLVESGGGLVKPGGSLKLSCAASGFTFSSYAMSWVRQTPEKRLEWVSSISSGGTTYYPDTVKGRFTISRDNARNILYLQMSSLRSEDTAMYYCARDSLYYGSSLYYAMDYWGQGTSVTVSS

SEQ ID NO: 189 - protein, 107 aa, synthetic construct
DIKMTQSPSSMYASLGERVSITCRASQDIYSYLSWFQQKPGKSPKTLIYRANRLVDGVPSRFSGSGSGQDYSLTISSLEYEDMGIYYCLQYDEFPYTFGGGTKLEIK

SEQ ID NO: 190 - protein, 5 aa, synthetic construct
SYAMS

SEQ ID NO: 191 - protein, 17 aa, synthetic construct
SISSGGTTYYPDTVKGR

SEQ ID NO: 192 - protein, 15 aa, synthetic construct
DSLYYGSSLYYAMDY

SEQ ID NO: 193 - protein, 11 aa, synthetic construct
RASQDIYSYLS

SEQ ID NO: 194 - protein, 8 aa, synthetic construct
RANRLVDG

SEQ ID NO: 195 - protein, 9 aa, synthetic construct
LQYDEFPYT

SEQ ID NO: 196 - protein, 120 aa, synthetic construct
EVQLQQSGPELVKPGASVKMSCKASGYTFTTYVMHWVKQKPGQGLEWIGYINPYNGGIRYNEKFKGKATLTSDKSSSTAYMELSSLTSEDSAVYYCARRERGVYYGMDEWGQGTSVTVSS

SEQ ID NO: 197 - protein, 112 aa, synthetic construct
DVVMTQTPLTLSVTIGQPASISCKSSQSLLHSNGKTYLNWLLQRPGQSPNLLIYLVSKLESGVPDRFSGSGSGTDFTLKISRVEAEDLGVYYCLQATYFPYTFGSGTKLEIK

SEQ ID NO: 198 - protein, 5 aa, synthetic construct
TYVMH

SEQ ID NO: 199 - protein, 17 aa, synthetic construct
YINPYNGGIRYNEKFKG

SEQ ID NO: 200 - protein, 11 aa, synthetic construct
RERGVYYGMDE

SEQ ID NO: 201 - protein, 16 aa, synthetic construct
KSSQSLLHSNGKTYLN

SEQ ID NO: 202 - protein, 8 aa, synthetic construct
LVSKLESG

SEQ ID NO: 203 - protein, 9 aa, synthetic construct
LQATYFPYT

SEQ ID NO: 204 - protein, 119 aa, synthetic construct
EVKLVESGPVLVAPSQSLSITCTVSGFSLTSYGVHWVRQPPGKGLEWLGVIWAGGHTNYNSDLMSRLSISKDNSKSQVFLKMNSLQTDDTAMYYCVKRSIYGDYALDYWGQGTSVTVSS

SEQ ID NO: 205 - protein, 106 aa, synthetic construct
DIVLTQSPAITAASLGQKVTITCSASSSVTYMHWYQQKSGTSPKLWIYDISKLASGVPARFSGSGPGTSYSLTISTMEAEDAAIYYCQQWNYPLMTFGGGTKLEIK

SEQ ID NO: 206 - protein, 5 aa, synthetic construct
SYGVH

SEQ ID NO: 207 - protein, 17 aa, synthetic construct
VIWAGGHTNYNSDLMSR

SEQ ID NO: 208 - protein, 11 aa, synthetic construct
RSIYGDYALDY

SEQ ID NO: 209 - protein, 5 aa, synthetic construct
VTYMH

SEQ ID NO: 210 - protein, 8 aa, synthetic construct
DISKLASG

SEQ ID NO: 211 - protein, 9 aa, synthetic construct
QQWNYPLMT

SEQ ID NO: 212 - protein, 119 aa, synthetic construct
QVQLQQPGAELVMPGASVKMSCKASGYKFTDYWMHWVKQRPGQGLEWIGAIDTSDSSTRNNQKFKGKATLTVDESSSTAYMQLSRLTSEDSAVYYCARGARTGTGFGYWGQGTLVTVSS

SEQ ID NO: 213 - protein, 112 aa, synthetic construct
DVVMTQTPLTLSVAIGQPASISCKSSQSLLHINGKTYLNWLLQRPGQSPKLLIYLVSKLESGVPDRFSGSGSGTDFTLKISRVEAEDLGVYYCLQATHFPYTFGSGTKLEIK

SEQ ID NO: 214 - protein, 5 aa, synthetic construct
DYWMH

SEQ ID NO: 215 - protein, 17 aa, synthetic construct
AIDTSDSSTRNNQKFKG

SEQ ID NO: 216 - protein, 10 aa, synthetic construct
GARTGTGFGY

SEQ ID NO: 217 - protein, 16 aa, synthetic construct
KSSQSLLHINGKTYLN

SEQ ID NO: 218 - protein, 621 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCLQATYFPYTFGQGTKLEIKGGGGSGGGGSGGGGSQVQLVQSGAEVKKPGASVKVSCKASGYTFTNYVMHWVRQAPGQGLEWMGYINPYSGGIRYNEKFKGRVTMTRDTSTSTVYMELSSLRSEDTAVYYCARRERGVYYGMDEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 219 - protein, 9 aa, synthetic construct
LQATHFPYT

SEQ ID NO: 220 - protein, 15 aa, synthetic construct
DALYYGSSLYYAMDY

SEQ ID NO: 221 - protein, 8 aa, synthetic construct
RANRLVDA

SEQ ID NO: 222 - protein, 123 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGSSLYYAMDYWGQGTLVTVSS

SEQ ID NO: 223 - protein, 107 aa, synthetic construct
DIQMTQSPSSLSASVGDRVTITCRASQDIYSYLSWFQQKPGKAPKTLIYRANRLVDAVPSRFSGSGSGQDYTLTISSLQPEDFATYYCLQYDEFPYTFGQGTKLEIK

SEQ ID NO: 224 - protein, 120 aa, synthetic construct
QVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMDEWGQGTLVTVSS

SEQ ID NO: 225 - protein, 112 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCLQATYFPYTFGQGTKLEIK

SEQ ID NO: 226 - protein, 123 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGGSLYYAMDYWGQGTLVTVSS

SEQ ID NO: 227 - protein, 15 aa, synthetic construct
DALYYGGSLYYAMDY

SEQ ID NO: 228 - protein, 123 aa, synthetic construct
EVQLVESGGGLVKPGGSLRLSCAASGFTFSRYAMSWVRQAPGKRLEWVSSISSGGNTYYPDTVKGRFTISRDNAKNSLYLQMNSLRAEDTAVYYCARDALYYGSSLYYAMDYWGQGTLVTVSS

SEQ ID NO: 229 - protein, 107 aa, synthetic construct
DIQMTQSPSSLSASVGDRVTITCRASQDIYSYLSWFQQKPGKAPKTLIYRENRLVDAVPSRFSGSGSGQDYTLTISSLQPEDFATYYCLQYDEFPYTFGQGTKLEIK

SEQ ID NO: 230 - protein, 10 aa, synthetic construct
GFTFSRYAMS

SEQ ID NO: 231 - protein, 17 aa, synthetic construct
SISSGGNTYYPDTVKGR

SEQ ID NO: 232 - protein, 8 aa, synthetic construct
RENRLVDA

SEQ ID NO: 233 - protein, 17 aa, synthetic construct
SISSGGTRYYPDTVKGR

SEQ ID NO: 234 - protein, 123 aa, synthetic construct
EVQLVESGGGLVKPGGSLRLSCAASGFTFRSYAMSWVRQAPGKRLEWVSSISSGGTRYYPDTVKGRFTISRDNAKNSLYLQMNSLRAEDTAVYYCARDALYYGSSLYYAMDYWGQGTLVTVSS

SEQ ID NO: 235 - protein, 120 aa, synthetic construct
QVQLVQSGAEVKKPGASVKVSCKASGYRFTTYVMHWVRQAPGQGLEWMGYINPYNGVIRYNEKFKGRVTMTRDTSTSTVYMELSSLRSEDTAVYYCARRERGVYYGMDEWGQGTLVTVSS

SEQ ID NO: 236 - protein, 112 aa, synthetic construct
DIVMTQTPLSLSVTPGQPASISCKSSQSLLHSNGKTYLNWYLQKPGQSPQLLIYLVSKLSSGVPDRFSGSGSGTDFTLKISRVEAEDVGVYYCLQATYFPYTFGQGTKLEIK

SEQ ID NO: 237 - protein, 214 aa, synthetic construct; identical to SEQ ID NO: 147

SEQ ID NO: 238 - protein, 17 aa, synthetic construct
YINPYNGVIRYNEKFKG

SEQ ID NO: 239 - protein, 8 aa, synthetic construct
LVSKLSSG

SEQ ID NO: 240 - protein, 120 aa, synthetic construct
QVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMSEWGQGTLVTVSS

SEQ ID NO: 241 - protein, 112 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCYQATYFPYTFGQGTKLEIK

SEQ ID NO: 242 - protein, 11 aa, synthetic construct
RERGVYYGMSE

SEQ ID NO: 243 - protein, 9 aa, synthetic construct
YQATYFPYT

SEQ ID NO: 244 - protein, 120 aa, synthetic construct
QVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVYYGMSEWGQGTLVTVSS

SEQ ID NO: 245 - protein, 112 aa, synthetic construct
DIVMTQTPLSLSVTPGQPASISCKSSQSLLHSNDKTYLNWYLQKPGQSPQLLIYLVSKLESGVPDRFSGSGSGTDFTLKISRVEAEDVGVYYCYQATYFPYTFGQGTKLEIK

SEQ ID NO: 246 - protein, 17 aa, synthetic construct
YINPYSGGIRYNEKFKG

SEQ ID NO: 247 - protein, 16 aa, synthetic construct
KSSQSLLHSNDKTYLN

SEQ ID NO: 248 - protein, 120 aa, synthetic construct
QVQLVQSGAEVKKPGASVKVSCKASGYTFTTYVMHWVRQAPGQGLEWMGYINPYSGGIRYNEKFKGRVTMTRDTSTSTVYMELSSLRSEDTAVYYCARRERGVTAGMDEWGQGTLVTVSS

SEQ ID NO: 249 - protein, 11 aa, synthetic construct
RERGVTAGMDE

SEQ ID NO: 250 - protein, 120 aa, synthetic construct
QVQLVQSGAEVKKPGASVKVSCKASGYTFTNYVMHWVRQAPGQGLEWMGYINPYSGGIRYNEKFKGRVTMTRDTSTSTVYMELSSLRSEDTAVYYCARRERGVYYGMDEWGQGTLVTVSS

SEQ ID NO: 251 - protein, 5 aa, synthetic construct
NYVMH

SEQ ID NO: 252 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 5 aa, synthetic construct
GSGGS

SEQ ID NO: 255 - protein, 234 aa, synthetic construct
AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 256 - protein, 234 aa, synthetic construct
AAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 257 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 258 - protein, 107 aa, synthetic construct [sequence omitted]

SEQ ID NO: 259 - protein, 120 aa, synthetic construct
QVQLVQSGAEVKKPGASVKVSCKASGYTFTTYVMHWVRQAPGQCLEWMGYINPYSGGIRYNEKFKGRVTMTRDTSTSTVYMELSSLRSEDTAVYYCARRERGVYYGMSEWGQGTLVTVSS

SEQ ID NO: 260 - protein, 112 aa, synthetic construct
DIVMTQTPLSLSVTPGQPASISCKSSQSLLHSNDKTYLNWYLQKPGQSPQLLIYLVSKLESGVPDRFSGSGSGTDFTLKISRVEAEDVGVYYCYQATYFPYTFGCGTKLEIK

SEQ ID NO: 261 - protein, 593 aa, synthetic construct
EVKLVESGGGLVKPGGSLKLSCAASGFTFSSYAMSWVRQTPEKRLEWVSSISSGGTTYYPDTVKGRFTISRDNARNILYLQMSSLRSEDTAMYYCARDSLYYGSSLYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 262 - protein, 214 aa, synthetic construct
DIKMTQSPSSMYASLGERVSITCRASQDIYSYLSWFQQKPGKSPKTLIYRANRLVDGVPSRFSGSGSGQDYSLTISSLEYEDMGIYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 590 aa, synthetic construct
EVQLQQSGPELVKPGASVKMSCKASGYTFTTYVMHWVKQKPGQGLEWIGYINPYNGGIRYNEKFKGKATLTSDKSSSTAYMELSSLTSEDSAVYYCARRERGVYYGMDEWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 264 - protein, 219 aa, synthetic construct
DVVMTQTPLTLSVTIGQPASISCKSSQSLLHSNGKTYLNWLLQRPGQSPNLLIYLVSKLESGVPDRFSGSGSGTDFTLKISRVEAEDLGVYYCLQATYFPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 589 aa, synthetic construct
QVQLQQPGAELVMPGASVKMSCKASGYKFTDYWMHWVKQRPGQGLEWIGAIDTSDSSTRNNQKFKGKATLTVDESSSTAYMQLSRLTSEDSAVYYCARGARTGTGFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 266 - protein, 219 aa, synthetic construct
DVVMTQTPLTLSVAIGQPASISCKSSQSLLHINGKTYLNWLLQRPGQSPKLLIYLVSKLESGVPDRFSGSGSGTDFTLKISRVEAEDLGVYYCLQATHFPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 589 aa, synthetic construct
EVKLVESGPVLVAPSQSLSITCTVSGFSLTSYGVHWVRQPPGKGLEWLGVIWAGGHTNYNSDLMSRLSISKDNSKSQVFLKMNSLQTDDTAMYYCVKRSIYGDYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 268 - protein, 213 aa, synthetic construct
DIVLTQSPAITAASLGQKVTITCSASSSVTYMHWYQQKSGTSPKLWIYDISKLASGVPARFSGSGPGTSYSLTISTMEAEDAAIYYCQQWNYPLMTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 593 aa, synthetic construct
EVKLVESGGGLVKPGGSLKLSCAASGFTFSSYAMSWVRQTPEKRLEWVSSISSGGTTYYPDTVKGRFTISRDNARNILYLQMSSLRSEDTAMYYCARDSLYYGSSLYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 270 - protein, 621 aa, synthetic construct
DVVMTQTPLTLSVTIGQPASISCKSSQSLLHSNGKTYLNWLLQRPGQSPNLLIYLVSKLESGVPDRFSGSGSGTDFTLKISRVEAEDLGVYYCLQATYFPYTFGSGTKLEIKGGGGSGGGGSGGGGSEVQLQQSGPELVKPGASVKMSCKASGYTFTTYVMHWVKQKPGQGLEWIGYINPYNGGIRYNEKFKGKATLTSDKSSSTAYMELSSLTSEDSAVYYCARRERGVYYGMDEWGQGTSVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 271 - protein, 214 aa, synthetic construct; identical to SEQ ID NO: 262

SEQ ID NO: 272 - protein, 855 aa, synthetic construct
DVVMTQTPLTLSVTIGQPASISCKSSQSLLHSNGKTYLNWLLQRPGQSPNLLIYLVSKLESGVPDRFSGSGSGTDFTLKISRVEAEDLGVYYCLQATYFPYTFGSGTKLEIKGGGGSGGGGSGGGGSEVQLQQSGPELVKPGASVKMSCKASGYTFTTYVMHWVKQKPGQGLEWIGYINPYNGGIRYNEKFKGKATLTSDKSSSTAYMELSSLTSEDSAVYYCARRERGVYYGMDEWGQGTSVTVSSGGGGSGGGGSGGGGSEVKLVESGGGLVKPGGSLKLSCAASGFTFSSYAMSWVRQTPEKRLEWVSSISSGGTTYYPDTVKGRFTISRDNARNILYLQMSSLRSEDTAMYYCARDSLYYGSSLYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 273 - protein, 214 aa, synthetic construct; identical to SEQ ID NO: 262

SEQ ID NO: 274 - protein, 593 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 275 - protein, 214 aa, synthetic construct; identical to SEQ ID NO: 147

SEQ ID NO: 276 - protein, 590 aa, synthetic construct
QVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMDEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 277 - protein, 219 aa, synthetic construct; identical to SEQ ID NO: 66

SEQ ID NO: 278 - protein, 593 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGSSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 279 - protein, 621 aa, synthetic construct
DIVMTQTPLSLSVTPGQPASISCKSSQSLLHSNGKTYLNWYLQKPGQSPQLLIYLVSKLESGVPDRFSGSGSGTDFTLKISRVEAEDVGVYYCLQATYFPYTFGQGTKLEIKGGGGSGGGGSGGGGSQVQLVQSGAEVKKPGASVKVSCKASGYTFTTYVMHWVRQAPGQGLEWMGYINPYNGGIRYNEKFKGRVTMTRDTSTSTVYMELSSLRSEDTAVYYCARRERGVYYGMDEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 280 - protein, 214 aa, synthetic construct; identical to SEQ ID NO: 147

SEQ ID NO: 281 - protein, 593 aa, synthetic construct
EVQLVESGGGLVKPGGSLRLSCAASGFTFSSYAMSWVRQAPGKRLEWVSSISSGGTTYYPDTVKGRFTISRDNAKNSLYLQMNSLRAEDTAVYYCARDALYYGGSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 282 - protein, 214 aa, synthetic construct; identical to SEQ ID NO: 147

SEQ ID NO: 283 - protein, 621 aa, synthetic construct
DIVMTQTPLSLSVTPGQPASISCKSSQSLLHSNDKTYLNWYLQKPGQSPQLLIYLVSKLESGVPDRFSGSGSGTDFTLKISRVEAEDVGVYYCYQATYFPYTFGCGTKLEIKGGGGSGGGGSGGGGSQVQLVQSGAEVKKPGASVKVSCKASGYTFTTYVMHWVRQAPGQCLEWMGYINPYSGGIRYNEKFKGRVTMTRDTSTSTVYMELSSLRSEDTAVYYCARRERGVYYGMSEWGQGTLVTVSSAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGKGGGGSGGGGSGGGGSQVQLQESGGGLVQPGGSLRLSCAASGYTYSSNCMGWFRQAPGKGLEGVAVICTGGGSPSYADSVKGRFTISRDNAKNTLYLQMNSLRAEDTAVYYCAADLLRAGTPLSSYEFNYWGQGTLVTVSS

SEQ ID NO: 284 - protein, 20 aa, synthetic construct
GGGGSGGGGSGGGGSGGGGS

SEQ ID NO: 285 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 287 - protein, 5 aa, synthetic construct
GGGGS

SEQ ID NO: 288 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 289 - protein, 222 aa, synthetic construct
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 290 - protein, 4 aa, synthetic construct
GGGS

SEQ ID NO: 291 - protein, 10 aa, synthetic construct
EPKSSDKTHT

(19) United States
(12) Patent Application Publication
(10) Pub. No.: US 2026/0078196 A1
(43) Pub. Date: Mar. 19, 2026

(54) MULTISPECIFIC CONSTRUCTS AND USES THEREOF

(71) Applicant: TJ BIOPHARMA (SHANGHAI) CO., LTD., Shanghai (CN)

(72) Inventors: Xi CHEN, Shanghai (CN); Jian LI, Shanghai (CN); Zhengyi WANG, Shanghai (CN)

(21) Appl. No.: 19/109,049

(22) PCT Filed: Sep. 6, 2023
(86) PCT No.: PCT/CN2023/117317

§ 371 Date: Mar. 5, 2025

(30) Foreign Application Priority Data
Sep. 6, 2022 (WO) PCT/CN2022/117334

(51) Int. Cl. C07K 16/28; A61K 39/00; A61P 35/00; C07K 16/30

(52) U.S. Cl. CPC C07K 16/2878; A61P 35/00; C07K 16/28; C07K 16/2803; C07K 16/3092; A61K 2039/505; C07K 2317/24; C07K 2317/31; C07K 2317/33; C07K 2317/73; C07K 2317/75; C07K 2317/92

(57) ABSTRACT

Provided are bispecific and multi-specific antibodies that include an agonist anti-4-1BB single-domain antibody capable of effector function-mediated tumor inhibition. In addition, these agonist antibodies, in the absence of tumor antigen-expressing cells, bind to 4-1BB but are unable to activate 4-1BB signaling. In the presence of a tumor antigen-expressing cell, however, these antibodies can trigger tumor antigen-dependent 4-1BB signaling, leading to potent immune response to the tumor antigen-expressing tumor cells.

### FIELD OF THE DISCLOSURE

The present application relates to multispecific molecules, e.g., anti-tumor antigen/anti-4-1BB bispecific antibodies and uses thereof including treating diseases or conditions.

### BACKGROUND

4-1BB (CD137, tumor necrosis factor receptor superfamily 9) is a member of TNF-receptor superfamily (TNFRSF) and is a costimulatory molecule which is expressed following the activation of immune cells, including both innate and adaptive immune cells. 4-1BB plays an important role in modulating the activity of immune cells. Agonists of 4-1BB can enhance immune cell proliferation, survival, secretion of cytokines and cytolytic activity CD8 T cells. Many studies showed that activation of 4-1BB enhances immune response to eliminate tumors in mice, indicating 4-1BB as a promising target molecule in cancer immunology.

The disclosures of all publications, patents, patent applications and published patent applications referred to herein are hereby incorporated herein by reference in their entirety.

### SUMMARY

Prior to the present disclosure, it was conventional knowledge that agonistic anti-4-1BB antibodies should not be ADCC-enabled. This is at least because ADCC-inducing FcγR binding could deplete 4-1BB+ cells, thereby undermining the antibody's agonistic effects and causing toxicities (e.g., liver toxicities) due to FcγR engagement. For instance, the leading anti-4-1BB antibody drug candidate urelumab includes an IgG4 Fc with no or limited effector function.

In an unexpected discovery, the instant inventors demonstrated that when a proprietary anti-4-1BB single domain antibody (represented by SEQ ID NO:27) was used in a bispecific format along with a second antibody moiety targeting a tumor-associated antigen (TAA), the effector function by the use of WT IgG1 actually enhanced its tumor inhibition activity. More specifically, in Experimental Example 4, two different versions of anti-claudin 6 (CLDN6-1)/anti-4-1BB bispecific antibodies were compared. One of them included a wild-type (WT) IgG1 Fc and the other with a silencing mutation (N297A or NA). As shown in FIG. 7B, while both the WT (with effector function) and the NA (no effector function) versions exhibited good anti-tumor activities, the WT version achieved significantly higher and lasting tumor inhibition.

Without being bound by a particular theory, such a unique effector function-assisted property of the bispecific antibody could be attributed to the activity or format (e.g., single-domain) of the instant anti-4-1BB antibody.

In accordance with one embodiment of the present disclosure, therefore, provided is a multispecific construct such as a multispecific antibody, comprising: (1) a first antibody moiety that specifically binds to a tumor antigen; and (2) a second antibody moiety that specifically binds to 4-1BB. In some embodiments, the second antibody moiety's 4-1BB activation activity is dependent upon the first antibody moiety's binding to the corresponding tumor antigen.

In some embodiments, the multispecific construct further comprises a Fc domain with maintained or improved effector function.

In some embodiments, the first antibody moiety is selected from the group consisting of a full-length antibody, a half-antibody, a single chain half-antibody, Fab, Fab′, F(ab′)₂, and scFv.

In some embodiments, the second antibody moiety specifically binds to CRD 3/4 region of 4-1BB. In some embodiments, the second antibody moiety is selected from the group consisting of a full-length antibody, a half-antibody, a single chain half-antibody, Fab, Fab′, F(ab′)₂, scFv, and sdAb. In some embodiments, the second antibody moiety is a sdAb. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27. In some embodiments, the CDR1, CDR2, and CDR3 are according to Kabat number scheme. In some embodiments, the sdAb comprises: (1) a sdAb-CDR1 comprising an amino acid sequence of SEQ ID NO: 24; (2) a sdAb-CDR2 comprising an amino acid sequence of SEQ ID NO: 25; and (3) a sdAb-CDR3 comprising an amino acid sequence of SEQ ID NO: 26. In some embodiments, the second antibody moiety comprises the amino acid sequence of SEQ ID NO:27, or a variant thereof having at least about 80% sequence identity to SEQ ID NO: 27.

In some embodiments, the multispecific construct is a bispecific antibody or a bispecific binding fragment.

In some embodiments, the multispecific construct further comprises an Fc domain. In some embodiments, the Fc domain is derived from anyone selected from the group consisting of IgG1, IgG2, IgG3, and IgG4. In some embodiments, the Fc domain is derived from IgG1. In some embodiments, the Fc domain comprises an amino acid sequence having at least 80% identity with any one of SEQ ID NOs: 46-56, 285-286 and 288-289.

In some embodiments, the activation of 4-1BB by the second antibody moiety is enhanced by at least 10 folds after binding of the first antibody moiety with the tumor antigen. In some embodiments, the activation of the 4-1BB by the second antibody moiety results in an increase in IFNγ level, IL-2 level, or NFκB signaling. In some embodiments, the first antibody moiety has a binding affinity of about 10⁻⁷ M to about 10⁻¹ M.

In some embodiments, the first antibody moiety is fused to the C-terminus of the second antibody moiety. In some embodiments, the first antibody moiety is fused to the N-terminus of the second antibody moiety. In some embodiments, the first antibody moiety and the second antibody moiety are fused to each other via a linker.

In some embodiments, the first antibody moiety is a Fab′ fused to N-terminus of the IgG Fc domain and the second antibody moiety is a sdAb fused to C-terminus of the IgG Fc domain. In some embodiments, the second antibody moiety is fused to the IgG Fc domain via a linker.

In some embodiments, the multispecific construct further comprises a third antibody moiety that specifically binds to a second tumor antigen. In some embodiments, the second tumor antigen is the same as the tumor antigen but has different epitope.

In some embodiments, the third antibody moiety is selected from the group consisting of a full-length antibody, a half-antibody, a single chain half-antibody, Fab, Fab′, F(ab′)₂, and scFv.

In some embodiments, first antibody moiety is a Fab′ fused to N-terminus of the IgG Fc domain and the second antibody moiety is a sdAb fused to C-terminus of the IgG Fc domain, and the third antibody moiety is a scFv fused to N-terminus of the first antibody moiety. In some embodiments, the second antibody moiety is fused to the IgG Fc domain via a linker, and the third antibody moiety is fused to the first antibody moiety via a linker.

In some embodiments, first antibody moiety is a Fab′ fused to N-terminus of a IgG Fc domain and the second antibody moiety is a sdAb fused to C-terminus of the IgG Fc domain, the third antibody moiety is a scFv fused to N-terminus of a pairing IgG Fc domain and the second antibody moiety is a sdAb fused to C-terminus of the pairing IgG Fc domain, wherein the pairing IgG Fc domain forms a heterodimer with the IgG Fc domain. In some embodiments, the second antibody moiety is fused to the IgG Fc domain via a linker, and the second antibody moiety is fused to the pairing IgG Fc domain via a linker. In certain embodiments, the linker comprises an amino acid sequence of SEQ ID NO: 23.

In some embodiments, the third antibody moiety is fused to the N-terminus of the pairing IgG Fc domain via a tether. The tether includes from N- to C-terminus a short linker “AA” and/or a hinge variant of “EPKSSDKTHT” (SEQ ID NO: 291).

In some embodiments, the Fc domain comprises an amino acid sequence of SEQ ID NO: 285 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 285, and the pairing Fc domain comprising an amino acid sequence of SEQ ID NO: 288 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 288.

In some embodiments, the Fc domain comprising an amino acid sequence of SEQ ID NO: 286, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 286, and the pairing Fc domain comprising an amino acid sequence of SEQ ID NO: 289 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 289.

In another aspect, provided herein is a pharmaceutical composition comprising the multispecific construct as described in the present application, and a pharmaceutically acceptable carrier.

In another aspect, provided herein is a nucleic acid encoding the multispecific construct as described in the present application.

In another aspect, provided is a vector comprising the nucleic acid as described in the present application.

In another aspect, provided herein is a host cell comprising the nucleic acid as described in the present application, or the vector as described in the present application.

In another aspect, provided herein is a method of treating a disease or condition in a subject in need thereof, comprising administering to the subject an effective amount of the multispecific construct as described in the present application, or the pharmaceutical composition as described in the present application. In some embodiments, the disease or condition is cancer.

In another aspect, provided herein is use of the multispecific construct as described in the present application in preparing a medicament for treating a disease or condition in a subject in need thereof.

The present disclosure also provides an antibody or antigen-binding fragment thereof having specificity to a human Mucin 16 (MUC16) protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein the HCDR1, HCDR2, HCDR3, LCDR1, LCDR2, and LCDR3 are selected from the group consisting of:

(a)
HCDR1:
(SEQ ID NO: 57)
SYAIS,
HCDR2:
(SEQ ID NO: 58)
GIIPIFGTANYAQKFQG,
HCDR3:
(SEQ ID NO: 59)
DSRKYYYDSSGPALWGFDAFDI,
LCDR1:
(SEQ ID NO: 60)
RASQSISSYLN,
LCDR2:
(SEQ ID NO: 61)
AASSLQS,
and
LCDR3:
(SEQ ID NO: 62)
QQSYSTLST,
(b)
HCDR1:
(SEQ ID NO: 57)
SYAIS,
HCDR2:
(SEQ ID NO: 58)
GIIPIFGTANYAQKFQG,
HCDR3:
(SEQ ID NO: 67)
EPPLSNYGDYATEQYYYGMDV,
LCDR1:
(SEQ ID NO: 60)
RASQSISSYLN,
LCDR2:
(SEQ ID NO: 61)
AASSLQS,
and
LCDR3:
(SEQ ID NO: 70)
QQSYSTPLT,
(c)
HCDR1:
(SEQ ID NO: 57)
SYAIS,
HCDR2:
(SEQ ID NO: 58)
GIIPIFGTANYAQKFQG,
HCDR3:
(SEQ ID NO: 75)
APMVRGVPPTPYYYYYGMDV,
LCDR1:
(SEQ ID NO: 76)
RASQSVSNYLA,
LCDR2:
(SEQ ID NO: 77)
DASNRAT,
and
LCDR3:
(SEQ ID NO: 78)
QQRSNWPS,
(d)
HCDR1:
(SEQ ID NO: 57)
SYAIS,
HCDR2:
(SEQ ID NO: 58)
GIIPIFGTANYAQKFQG,
HCDR3:
(SEQ ID NO: 83)
TPELLWFGELGGAYYFDY,
LCDR1:
(SEQ ID NO: 84)
RASESISSWLA,
LCDR2:
(SEQ ID NO: 85)
KASTLEN,
and
LCDR3:
(SEQ ID NO: 86)
QQYRSHWSST,
(e)
HCDR1:
(SEQ ID NO: 57)
SYAIS,
HCDR2:
(SEQ ID NO: 58)
GIIPIFGTANYAQKFQG,
HCDR3:
(SEQ ID NO: 91)
ANFNIYYYYYGMDV,
LCDR1:
(SEQ ID NO: 92)
RSSQSLLHSNGYNYLD,
LCDR2:
(SEQ ID NO: 93)
LGSNRAS,
and
LCDR3:
(SEQ ID NO: 94)
MQGTHWPRT,
(f)
HCDR1:
(SEQ ID NO: 97)
SYEMN,
HCDR2:
(SEQ ID NO: 98)
RIKSKTDGGTTDYAAPV,
HCDR3:
(SEQ ID NO: 99)
DLAAVAGLFDY,
LCDR1:
(SEQ ID NO: 100)
QASQDISNYLN,
LCDR2:
(SEQ ID NO: 101)
DASNLET,
and
LCDR3:
(SEQ ID NO: 102)
QQSYSTPWK,
(g)
HCDR1:
(SEQ ID NO: 57)
SYAIS,
HCDR2:
(SEQ ID NO: 106)
RIIPIFGIANYAQKFQG,
HCDR3:
(SEQ ID NO: 107)
TGDYDILTGSYYYGMDV,
LCDR1:
(SEQ ID NO: 108)
RASQGIRNDLG,
LCDR2:
(SEQ ID NO: 61)
AASSLQS,
and
LCDR3:
(SEQ ID NO: 120)
LQDYNYPFT,
(h)
HCDR1:
(SEQ ID NO: 123)
DYYLS,
HCDR2:
(SEQ ID NO: 58)
GIIPIFGTANYAQKFQG,
HCDR3:
(SEQ ID NO: 125)
GGPHYDFWSGYTPGQHGGAFDI,
LCDR1:
(SEQ ID NO: 126)
RASQSVSSSYLA,
LCDR2:
(SEQ ID NO: 127)
GASSRAT,
and
LCDR3:
(SEQ ID NO: 128)
QQRSNWRNT,
(i)
HCDR1:
(SEQ ID NO: 57)
SYAIS,
HCDR2:
(SEQ ID NO: 58)
GIIPIFGTANYAQKFQG,
HCDR3:
(SEQ ID NO: 133)
DSGSSITMVRGGDYYYMDV,
LCDR1:
(SEQ ID NO: 134)
RASQSVSSYLA,
LCDR2:
(SEQ ID NO: 77)
DASNRAT,
and
LCDR3:
(SEQ ID NO: 136)
QQRSNWPPT,
(j)
HCDR1:
(SEQ ID NO: 139)
YHAIS,
HCDR2:
(SEQ ID NO: 140)
GIIPILGTANYAQKFQG,
HCDR3:
(SEQ ID NO: 141)
GTTAARYYYYYYYMDV,
LCDR1:
(SEQ ID NO: 100)
QASQDISNYLN,
LCDR2:
(SEQ ID NO: 101)
DASNLET,
and
LCDR3:
(SEQ ID NO: 144)
QQYDNLPLT,
(k)
HCDR1:
(SEQ ID NO: 57)
SYAIS,
HCDR2:
(SEQ ID NO: 58)
GIIPIFGTANYAQKFQG,
HCDR3:
(SEQ ID NO: 149)
SITDYYDSSGYYFRPHENTGYYYGMDV,
LCDR1:
(SEQ ID NO: 150)
RASQGINNYLA,
LCDR2:
(SEQ ID NO: 151)
AASTLQS,
and
LCDR3:
(SEQ ID NO: 152)
QQYDTFSET,
(l)
HCDR1:
(SEQ ID NO: 57)
SYAIS,
HCDR2:
(SEQ ID NO: 58)
GIIPIFGTANYAQKFQG,
HCDR3:
(SEQ ID NO: 125)
GGPHYDFWSGYTPGQHGGAFDI,
LCDR1:
(SEQ ID NO: 158)
RASQSISGWLA,
LCDR2:
(SEQ ID NO: 159)
RTSYLES,
and
LCDR3:
(SEQ ID NO: 160)
QHYDTFSRA,
or
(m)
HCDR1:
(SEQ ID NO: 139)
YHAIS,
HCDR2:
(SEQ ID NO: 38)
SISSGGNTYYPDTVKGR,
HCDR3:
(SEQ ID NO: 165)
EGPDYGDYSWSMDYYYGMDV,
LCDR1:
(SEQ ID NO: 166)
RASQSVNSRYLA,
LCDR2:
(SEQ ID NO: 167)
GASTRAT,
and
LCDR3:
(SEQ ID NO: 168)
QQYGTFSIT.

In some embodiments, antibody or antigen-binding fragment thereof provided herein comprises a heavy chain variable region comprising an amino acid sequence selected from the group consisting of SEQ ID NOs: 63, 71, 79, 87, 95, 103, 121, 129, 137, 145, 153, 161, and 169, or a peptide having at least 90% sequence identity to an amino acid sequence selected from the group consisting of SEQ ID NOs: 63, 71, 79, 87, 95, 103, 121, 129, 137, 145, 153, 161, and 169.

In some embodiments, antibody or antigen-binding fragment thereof provided herein comprises a light chain variable region comprising an amino acid sequence selected from the group consisting of SEQ ID NOs: 64, 72, 80, 88, 96, 104, 122, 130, 138, 146, 154, 162, and 170 or a peptide having at least 90% sequence identity to an amino acid sequence selected from the group consisting of SEQ ID NOs: 64, 72, 80, 88, 96, 104, 122, 130, 138, 146, 154, 162, and 170.

In some embodiments, In some embodiments, antibody or antigen-binding fragment thereof provided herein comprises
(a) a heavy chain variable region comprising the amino acid sequence of SEQ ID NO: 63, and a light chain variable region comprising the amino acid sequence of SEQ ID NO: 64;
(b) a heavy chain variable region comprising the amino acid sequence of SEQ ID NO: 71, and a light chain variable region comprising the amino acid sequence of SEQ ID NO: 72;
(c) a heavy chain variable region comprising the amino acid sequence of SEQ ID NO: 79, and a light chain variable region comprising the amino acid sequence of SEQ ID NO: 80;
(d) a heavy chain variable region comprising the amino acid sequence of SEQ ID NO: 87, and a light chain variable region comprising the amino acid sequence of SEQ ID NO: 88;
(e) a heavy chain variable region comprising the amino acid sequence of SEQ ID NO: 95, and a light chain variable region comprising the amino acid sequence of SEQ ID NO: 96;
(f) a heavy chain variable region comprising the amino acid sequence of SEQ ID NO: 103, and a light chain variable region comprising the amino acid sequence of SEQ ID NO: 104;
(g) a heavy chain variable region comprising the amino acid sequence of SEQ ID NO: 121, and a light chain variable region comprising the amino acid sequence of SEQ ID NO: 122;
(h) a heavy chain variable region comprising the amino acid sequence of SEQ ID NO: 129, and a light chain variable region comprising the amino acid sequence of SEQ ID NO: 130;
(i) a heavy chain variable region comprising the amino acid sequence of SEQ ID NO: 137, and a light chain variable region comprising the amino acid sequence of SEQ ID NO: 138;
(j) a heavy chain variable region comprising the amino acid sequence of SEQ ID NO: 145, and a light chain variable region comprising the amino acid sequence of SEQ ID NO: 146;
(k) a heavy chain variable region comprising the amino acid sequence of SEQ ID NO: 153, and a light chain variable region comprising the amino acid sequence of SEQ ID NO: 154;
(l) a heavy chain variable region comprising the amino acid sequence of SEQ ID NO: 161, and a light chain variable region comprising the amino acid sequence of SEQ ID NO: 162; or
(m) a heavy chain variable region comprising the amino acid sequence of SEQ ID NO: 169, and a light chain variable region comprising the amino acid sequence of SEQ ID NO: 170.

In some embodiments, the antibody is a chimeric antibody or a humanized antibody.

In some embodiments, the antibody or antigen-binding fragment thereof provided herein further comprising a heavy chain constant region, a light chain constant region, an Fc region, or the combination thereof.

In some embodiments, the antibody or antigen-binding fragment thereof provided herein comprising a first antibody moiety having specificity to a human MUC16 protein and a second antibody moiety having specificity to a second protein, wherein the first antigen-binding moiety comprises an antibody or antigen-binding fragment thereof provided herein.

In some embodiments, the second protein is 4-1BB. In some embodiments, the first antibody moiety is a full-length antibody. In some embodiments, the second antibody moiety is a sdAb. In some embodiments, the second antibody moiety is fused to C-terminus of the first antibody moiety. In some embodiments, the first antibody moiety and the second antibody moiety are fused to each other via a linker. In some embodiments, the second antibody moiety comprises HCDR1 of SNCMG (SEQ ID NO: 24), HCDR2 ofVICTGGGSPSYADSVKG (SEQ ID NO: 25), and HCDR3 of DLLRAGTPLSSYEFNY (SEQ ID NO: 26). In some embodiments, the second antibody moiety comprises an amino acid sequence of SEQ ID NO: 27 or a peptide having at least 90% sequence identity to the amino acid sequence of SEQ ID NO: 27.

In one aspect, the present disclosure provides a composition comprising the antibody or antigen-binding fragment thereof provided herein or the bifunctional molecule provided herein, and a pharmaceutically acceptable carrier.

In one aspect, the present disclosure provides an isolated cell comprising one or more polynucleotide encoding the antibody or antigen-binding fragment thereof provided herein or the bifunctional molecule provided herein.

In one aspect, the present disclosure provides a polynucleotide encoding one or more chains of the antibody or antigen-binding fragment thereof provided herein or the bifunctional molecule provided herein.

In one aspect, the present disclosure provides a method of treating a cancer in a patient in need thereof, comprising administering to the patient the antibody or antigen-binding fragment thereof provided herein or the bifunctional molecule provided herein.

In some embodiments, the cancer is selected from the group consisting of ovarian cancer, prostate cancer, cancer of the urinary tract, pancreatic cancer, lung cancer, breast cancer, bladder cancer, colorectal cancer, endometrial cancer, esophageal cancer, head and neck cancer, kidney cancer, leukemia, liver cancer, lymphoma, melanoma, and thyroid cancer.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1C illustrate binding affinity of CLDN6×4-1BB bispecific antibodies (BsAbs) disclosed herein with CLDN6.

FIGS. 2A-2C illustrate binding affinity of CLDN6×4-1BB BsAbs disclosed herein with CLDN6 expressing cells.

FIGS. 3A-3D illustrate binding affinity of CLDN6×4-1BB BsAbs disclosed herein with 4-1BB.

FIGS. 4A-4B illustrate binding of CLDN6×4-1BB BsAbs disclosed herein with soluble 4-1BB and cells expressing 4-1BB.

FIGS. 5A-5D illustrate CLDN6-dependent 4-1BB activation of CLDN6×4-1BB BsAbs disclosed herein.

FIGS. 6A-6H illustrate activation of PBMC by CLDN6×4-1BB BsAbs disclosed herein.

FIGS. 7A-7B illustrate tumor inhibition in hu4-1BB mice after treatment with CLDN6×4-1BB BsAbs disclosed herein.

FIGS. 8A-8B illustrate liver function after treatment of the CLDN6×4-1BB BsAbs of the present application.

FIGS. 9A-9C illustrate binding activity of the 13 selected monoclonal antibodies to human MUC16 protein as measured by ELISA.

FIG. 10 illustrates binding activity of the 13 selected monoclonal antibodies to cynomolgus monkey MUC16 protein as measured by ELISA.

FIGS. 11A-11C illustrate cell-based binding of the 13 selected monoclonal antibodies to human MUC16 positive and negative cells.

FIGS. 12A-12C illustrate cell-based binding activity of the 13 selected monoclonal antibodies to human MUC16 in OVCAR3, SNU216 and MUC16 overexpressed HEK293 cells, respectively.

FIGS. 13A-13D illustrate cell-based binding activity of 5 selected monoclonal antibodies to human MUC16 in OVCAR3 cell lines in the presence or absence of CA125.

FIGS. 14A-14B illustrate the kinetics of the binding activity of 3 selected monoclonal antibodies to human MUC16.

FIGS. 15A-15E illustrate MUC16-dependent 4-1BB activation in a 4-1BB NFκB reporter assay for cell lines with different MUC16 expression levels.

FIGS. 16A-16F illustrate MUC16-dependent 4-1BB activation for cell lines with different MUC16 expression levels, which induces T cell costimulatory activity in the PBMCs to release human IFNγ and IL-2 cytokine.

FIGS. 17A-17F illustrate MUC16-dependent 4-1BB activation for cell lines with different MUC16 expression levels, which induces T cell costimulatory activity in the CD8+ T cells to release human IFNγ and IL-2 cytokine.

FIGS. 18A-18B show the cellular binding activity of the bi-specific anti-ROR1/anti-4-1BB antibodies as measured by FACS.

FIGS. 19A-19C show the ROR1 dependent 4-1BB activation of the bi-specific anti-ROR1/anti-4-1BB antibodies in a reporter gene assay.

FIGS. 20A-20C show the epitope binning of the anti-ROR1 antibodies. A. The epitope binning workflow; B-C. Epitope binning results of 3C5 and 8F5, respectively.

FIGS. 21A-21C show the ROR1 dependent 4-1BB activation of the bi-paratopic anti-ROR1/anti-4-1BB antibodies in a reporter gene assay.

FIGS. 22A-22D show (A-B) the cellular binding activity to ROR1 of the anti-ROR1 mAb as measured by FACS, before and after humanization, and (C-D) ROR1 dependent 4-1BB activation in a reporter gene assay of the bi-specific anti-ROR1/anti-4-1BB antibodies, before and after humanization.

FIGS. 23A-23F show (A-C) cellular binding activity of the bi-specific and bi-paratopic anti-ROR1/anti-4-1BB antibodies as measured by FACS and (D-F) ROR1 dependent 4-1BB activation of the bi-specific and bi-paratopic anti-ROR1/anti-4-1BB antibodies in a reporter gene assay using ROR1 positive and negative tumor cell lines.

FIGS. 24A-24C show SPR results of the humanized bi-specific and bi-paratopic anti-ROR1/anti-4-1BB antibodies.

FIGS. 25A-25B show the in vivo efficacy study of the humanized bi-specific and bi-paratopic anti-ROR1/anti-4-1BB antibodies. (A) study design; (B) in vivo anti-tumor efficacy in 4-1BB knock-in mice.

FIGS. 26A-26E show the ROR1 dependent 4-1BB activation of the affinity matured bi-specific anti-ROR1/anti-4-1BB antibodies in a reporter gene assay.

FIGS. 27A-27C show the ROR1 dependent 4-1BB activation of the affinity matured bi-paratopic anti-ROR1/anti-4-1BB antibodies in a reporter gene assay.

FIGS. 28A-28B show the ROR1 dependent 4-1BB induced cytokine release of the affinity matured bi-specific and bi-paratopic anti-ROR1/anti-4-1BB antibodies in a reporter gene assay.

FIGS. 29A-29C show the in vivo efficacy study of the affinity matured bi-paratopic anti-ROR1/anti-4-1BB antibodies. (A) study design; (B) in vivo anti-tumor efficacy in 4-1BB knock-in C57 mice inoculated with hROR1 expressing MC38 cells; (C) summarized tumor growth inhibition (TGI) for each treatment group.

### DETAILED DESCRIPTION OF THE APPLICATION

### Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (73 heavy chain), IgG4 (γ4 heavy chain), IgA1 (α1 heavy chain), or IgA2 (α2 heavy chain).

The term “half antibody” as used herein refers to one immunoglobulin heavy chain associated with one immunoglobulin light chain. One skilled in the art will readily appreciate that a half-antibody may encompass a fragment thereof and may also have an antigen binding domain consisting of a single variable domain, e.g., originating from a camelidae. In certain embodiments, the half antibody comprises a Fab′ and a Fc domain. The Fab′ is fused to N-terminus of the Fc domain.

The term “single chain half-antibody” as used herein refers to a single chain polypeptide comprising a VL domain, optionally a CL domain, a tether, a VH domain, optionally a CH1 domain, a hinge domain, a CH2 domain and a CH3 domain, wherein said domains are positioned relative to each other in an N-terminal to C-terminal direction as follows: VL-tether-VH-hinge-CH2-CH3, VL-tether-VH-partial hinge-CH2-CH3, VL-tether-VH-hinge variant -CH2-CH3, or VL-CL-tether-VH-CH1-hinge-CH2-CH3.

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870). When the sdAb contains only a heavy chain, it can be exchangable used with “VHH” or “single heavy chain variable domain antibody” or “nanobody”.

The term “antigen-binding fragment” as used herein refers to an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody (e.g., a camelized single domain antibody), a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

A “Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

A “Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

A “F(ab)₂” refers to a dimer of Fab′ that comprises two light chains and part of the two heavy chains.

“Single-chain Fv,” also abbreviated as “sFv” or “scFv,” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra with minor modification. Briefly, we added 5 more residues in super variable loop before the heavy chain CDR1. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C3” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region”, “Fc domain” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof.

The C-terminal lysine (residue 447 according to the EU numbering system) of the Fc region may be removed, for example, during production or purification of the antibody, or by recombinantly engineering the nucleic acid encoding a heavy chain of the antibody.

Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As use herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or 10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BIACORE™-tests and peptide scans.

An “isolated” antibody (or construct) is one that has been identified, separated and/or recovered from a component of its production environment (e.g., natural or recombinant). Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The terms “agonizing” or “agonize” refer to an increase or enhancement of any phenotypic characteristic, or the incidence, degree, or likelihood of that characteristic. To “increase” or “enhance” is to decrease, reduce or arrest an activity, function, and/or amount as compared to that of a reference. In certain embodiments, by “increase” or “enhance” is meant the ability to cause an overall increase in, e.g., activity, function, and/or amount, of at least about 1-fold or greater. In another embodiment, by “increase” or “enhance” is meant the ability to cause an overall increase in, e.g., activity, function, and/or amount, of at least about 5-fold or greater as compared to a reference. In yet another embodiment, by “increase” or “enhance” is meant the ability to cause an overall increase in, e.g., activity, function, and/or amount, of at least about any one of 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold, as compared to a reference, including any range in between these values, or greater than about 100-fold, as compared to a reference.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

As used herein, “based upon” includes assessing, determining, or measuring the individual's characteristics as described herein (and preferably selecting an individual suitable for receiving treatment). When the status of a claudin-18 aberration is “used as a basis” for selection, assessing, measuring, or determining method of treatment as described herein, the CLDN6 aberration determined before and/or during treatment, and the status (including presence, absence, expression level, activity level and/or phosphorylation level of CLDN6) obtained is used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; or (g) predicting likelihood of clinical benefits.

The terms “subject,” “individual,” and “patient” are used interchangeably herein to refer to a mammal, including, but not limited to, human, bovine, horse, feline, canine, rodent, or primate. In some embodiments, the individual is a human.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### Antibody Binding Affinity

Binding specificity of the antibody moieties of the multispecific constructs described herein can be determined experimentally by methods known in the art. Such methods comprise, but are not limited to Western blots, ELISA-, RIA-, ECL-, IRMA-, EIA-, BIACORE™-tests and peptide scans.

In some embodiments, the binding affinity is measured by the dissociation constant, K_{D}. Dissociation constants may be determined through any analytical technique known in the art, including biochemical or biophysical techniques such as fluorescent activated cell sorting (FACS), flow cytometry, enzyme-linked immunosorbent assay (ELISA), surface plasmon resonance (SPR), BioLayer interferometry (see, e.g., Octet System by ForteBio), meso scale discover assays (see, e.g., MSD-SET), isothermal titration calorimetry (ITC), differential scanning calorimetry (DSC), circular dichroism (CD), stopped-flow analysis, and colorimetric or fluorescent protein melting analyses; or a cell binding assay.

In some embodiments, the K_{D} of the binding between the antibody moiety and the 4-1BB is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻¹ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and the 4-1BB is stronger than about any one of 10⁻⁷ M, 10⁻⁸ M, 10⁻⁹ M, 10⁻¹⁰ M, 10⁻¹¹ M, 10⁻² or 10⁻¹³ M. In some embodiments, the 4-1BB is a human antigen.

In some embodiments, the Kₒₙ of the binding between the antibody moiety and the 4-1BB is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and the 4-1BB is about 10³ M⁻¹s⁻¹ to about 10⁵ M^{-l}s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and the 4-1BB is no more than about any one of 10³ M⁻¹s⁻¹, 10⁴ M⁻¹s^{-1, 10}^{⁵} M⁻¹s⁻¹, 10⁶ M⁻¹s⁻¹, 10⁷ M⁻¹s⁻¹ or 10⁸ M⁻¹s⁻¹. In some embodiments, the 4-1BB is human antigen.

In some embodiments, the K_{off} of the binding between the antibody moiety and the 4-1BB is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻³ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s¹ to about 10⁻⁵ s⁻¹, or about 10⁻³ s⁻¹ to about 10⁻⁵ s⁻¹. In some embodiments, the K_{off} of the binding between the antibody moiety and the 4-1BB is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, the 4-1BB is human antigen.

In some embodiments, the K_{D} of the binding between the antibody moiety and the tumor antigen is about 10⁻⁷ M to about 10⁻² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹ M, about 10⁻¹ M to about 10⁻² M, about 10⁻⁷ M to about 10⁻² M, about 10⁻⁸ M to about 10⁻² M, about 10⁻⁹ M to about 10⁻² M, about 10⁻¹⁰ M to about 10⁻² M, about 10⁻⁸ M to about 10⁻¹ M, about 10⁻⁸ M to about 10⁻¹¹ M, about 10⁻⁹ M to about 10⁻¹² M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and the tumor antigen is stronger than about any one of 10⁻⁷ M, 10⁻⁸ M, 10⁻⁹ M, 10⁻¹⁰ M, 10⁻¹¹ M, or 10⁻¹² M. In some embodiments, the tumor antigen is a human antigen.

In some embodiments, the Kₒₙ of the binding between the antibody moiety and the tumor antigen is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M^{-l}s⁻¹, or about 10⁷ M⁻¹s⁻¹ to about 10⁸ M⁻¹s⁻¹. In some embodiments, the Kₒₙ of the binding between the antibody moiety and the tumor antigen is about 10³ M⁻¹s⁻¹ to about 10⁵ M⁻¹s⁻¹, about 10⁴ M⁻¹s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M^{-l}s⁻¹ to about 10⁸ M⁻¹s⁻¹. In some embodiments, the Kₒₙ of the binding between the antibody moiety and the tumor antigen is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹. In some embodiments, the tumor antigen is human antigen.

In some embodiments, the K_{off} of the binding between the antibody moiety and the tumor antigen is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 10⁻⁴s⁻¹ to about 10⁻⁶ s⁻¹, about 10⁻² s⁻¹ to about 10⁻⁶ s⁻¹, about 10⁻³ s⁻¹ to about 10⁻⁶ s⁻¹ about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹.

In some embodiments, the K_{off} of the binding between the antibody moiety and the tumor antigen is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, the tumor antigen is human antigen.

### Chimeric or Humanized Antibodies

In some embodiments, one or more of the antibody moieties of the multispecific constructs of the present application is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from mouse) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In some embodiments, one or more of the antibody moieties of the multispecific constructs of the present application is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

### Anti-MUC16 Antibodies

MUC 16 is a tumor associated antigen polypeptide, expressed by the human ocular surface epithelia in the mucosa of the bronchus, fallopian tube, and uterus. One proposed function of MUC 16 is to provide a protective, lubricating barrier against particles and infectious agents at mucosal surfaces. Highly polymorphic, MUC 16 is composed of three domains, a Ser-/Thr-rich N-terminal domain, a repeat domain of between eleven and more than 60 partially conserved tandem repeats of on average 156 amino acids each, and a C-terminal non-repeating domain containing a transmembrane sequence and a short cytoplasmic tail. MUC 16 is heavily 0-glycosylated and N-glycosylated. MUC16 is reported to be heavily overexpressed in certain types of human cancerous ovarian, breast and pancreatic tumors as compared to the corresponding normal human ovarian, breast and pancreatic tissues, respectively. Due to its overexpression in certain human tumors, the MUC 16 polypeptide and the nucleic acid encoding that polypeptide are targets for quantitative and qualitative comparisons among various mammalian tissue samples. The unique expression profiles of MUC 16 polypeptide, and the nucleic acid encoding that polypeptide, can be exploited for the diagnosis and therapeutic treatment of certain types of cancerous tumors in mammals.

The present disclosure provides antibodies, including antibodies or antigen-binding fragments thereof, that have binding specificity to the human MUC16 protein. As demonstrated in the experimental examples, 13 anti-human MUC16 antibodies were obtained, having high binding affinity to the human MUC16 protein. The antibody clones, D57, B218, C25 and D100, were selected for further multispecific antibody construction. The human antibodies bound human MUC16 with high affinity and the bispecific antibodies efficiently induced MUC16 dependent 4-1BB activation in T cells.

In accordance with one embodiment of the present disclosure, provided are antibodies or antigen-binding fragments thereof that include the heavy chain and light chain variable domains with the CDR regions of the antibodies prepared in the experimental examples. The CDRs and variable regions are summarized in Table 4 of the EXAMPLES (Kabat numbering).

In some embodiments, the VH CDR1, CDR2, and CDR3 are selected from any set of VH CDR1, CDR2, and CDR3 shown in Table 1, and the VL CDR1, CDR2, and CDR3 are selected from any set of VL CDR1, CDR2, and CDR3 shown in Table 1. In some embodiments, the VH CDR1, CDR2, and CDR3 and the VL CDR1, CDR2, and CDR3 are selected from those derived from the same antibody in the examples.

In some embodiments, at least one, or two, or three, or four, or five, or six of the VH CDR1, CDR2, and CDR3 and the VL CDR1, CDR2, and CDR3 of the above are modified by one, two or three amino acid additions, deletions, substitutions, or the combinations thereof.

The CDRs, heavy chain variable regions and light chain variable regions of the present disclosure can be further modified. In some embodiments, the modified heavy chain variable region or light chain variable region retains at least about 70%, 75%, 80%, 85%, 90%, 95%, 98% or 99% sequence identity and is still capable of binding to MUC16.

In some embodiments, the modification is substitution at no more than one hot spot position from each of the CDRs. In some embodiments, the modification is substitution at one, two or three such hot spot positions. In one embodiment, the modification is substitution at one of the hot spot positions. Such substitutions, in some embodiments, are conservative substitutions.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody provided herein further comprises a heavy chain constant region, a light chain constant region, an Fc region, or the combination thereof.

In certain embodiments, the present disclosure provides a bifunctional molecule, comprising a first antigen-binding portion having specificity to a human MUC16 protein and a second portion having specificity to a second protein, wherein the first antigen-binding portion comprises an anti-MUC16 antibody or fragment thereof as provided herein.

In certain embodiments, the second portion is an antibody or an antigen binding fragment thereof. In certain embodiments, the second portion has specificity to immune checkpoints. In certain embodiments, the second portion has specificity to other tumor antigens.

In certain embodiments, the second portion is a 4-1BB antigen-binding moiety.

In certain embodiments, the first antigen-binding portion provided herein has an full-length antibody/IgG format and the second antigen-binding moiety provided herein has a single domain antibody (sdAb) format.

In certain embodiments, the sdAb is fused to N-terminus or C-terminus of the IgG. In certain embodiments, the sdAb is fused to N-terminus of the IgG heavy chain variable region.

In certain embodiments, the sdAb is fused to C-terminus of the IgG heavy chain constant region (IgG(CH)) that comprises a Fc domain.

In certain embodiments, the multi-specific construct provided herein include a first polypeptide comprising from N- to C-terminus: VH(MUC16)-IgG(CH)-VHH(4-1BB), and a second polypeptide comprising from N- to C-terminus: VL(MUC16)-IgG light chain constant region (IgG(CL)). The first and second polypeptides are paired via the VH(MUC16)-VL(MUC16) pairing.

In certain embodiments, the multi-specific construct provided herein include two first polypeptides comprising from N- to C-terminus: VH(MUC16)-IgG(CH)-VHH(4-1BB), and two second polypeptides comprising from N- to C-terminus: VL(MUC16)-IgG light chain constant region (IgG(CL)). The first and second polypeptides are paired via the VH(MUC16)-VL(MUC16) pairing. The two first polypeptides can be paired via the IgG(CH) pairing.

The first antigen-binding portion and the second antigen-binding portion are fused via a linker.

### Substitution, Insertion, and Deletion Variants

In some embodiments, antibody variants comprising one or more amino acid substitutions are included in the multispecific constructs or antibodies described herein. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs. In some embodiments of the variant VHH sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In some embodiments, one or more antibody moieties of the multispecific construct or antibodies of the present application is altered to increase or decrease the extent to which the construct is glycosylated. Addition or deletion of glycosylation sites to an antibody may be conveniently accomplished by altering the amino acid sequence such that one or more glycosylation sites is created or removed.

Where the antibody moiety comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the antibody moiety has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the antibody moiety has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fc Domain

In some embodiments, the first antibody moiety or the second antibody moiety comprises an Fc region (also referred to herein as “Fc fragment”). In some embodiments, the Fc region is an Fc domain, namely, an Fc region possessing some or all effector functions, including for example complement-dependent cytotoxicity (CDC) and antibody-dependent cell mediated cytotoxicity (ADCC) functions. In some embodiments, the Fc domain is derived from IgG1 or IgG3.

In some embodiments, the Fc region is maintained or improved with effector function, such as ADCC and/or CDC.

In some embodiments, one or more amino acid modifications may be introduced into the Fc domain, thereby generating an Fc domain variant. The Fc domain variant may comprise a human Fc domain sequence (e.g., derived from human IgG1, IgG2, IgG3 or IgG4 Fc region) comprising an amino acid modification (e.g. a substitution) at one or more amino acid positions.

In some embodiments, the Fc domain variant change one or more functional and/or pharmacokinetic properties of the antibody.

In some embodiments, the Fc domain possesses some but not all effector functions, which make it a desirable candidate for applications in which the half-life of the antibody moiety in vivo is important yet certain effector functions (such as CDC and ADCC) are unnecessary or deleterious.

In vitro and/or in vivo cytotoxicity assays can be conducted to analyze CDC and/or ADCC activities of the Fe region. For example, Fe receptor (FcR) binding assays can be conducted to determine whether the antibody possesses FcγR binding (hence likely ADCC activity), and/or retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fe mutant with substitution of residues 265 and 297 to alanine (U.S. Pat. No. 7,332,581). In some embodiments, the Fc region of the multispecific construct does not comprises a mutation that reduces its effector function, such as one or more mutations described herein. In some embodiments, the Fc region of the multispecific construct comprises one or more of these mutations.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, the Fc region of the multispecific construct does not comprise a variant that with improved or diminished binding to FcRs. In some embodiments, the Fc region of the multispecific construct comprises a variant with improved binding to FcγRI. In some embodiments, the Fc region of the multispecific construct comprises a variant with improved binding to FcγRII. In some embodiments, the Fe region of the multispecific construct comprises a variant with improved binding to FcγRIII. In some embodiments, the Fc region of the multispecific construct comprises a variant with diminished binding to FcγRI. In some embodiments, the Fe region of the multispecific construct comprises a variant with diminished binding to FcγRII. In some embodiments, the Fc region of the multispecific construct comprises a variant with diminished binding to FcγRIII.

In some embodiments, the Fc domain is derived from human IgG1. In some embodiments, the human IgG1 derived Fc domain does not comprise L234A mutation and/or a L235A mutation. In some embodiments, the human IgG1 derived Fc domain comprises a L234A mutation and/or a L235A mutation. In some embodiments, the Fc domain is derived from human IgG3. In some embodiments, the Fc domain is derived from human IgG2 or IgG4. In some embodiments, the Fc domain is derived from human IgG4. In some embodiments, the human IgG4 derived Fc domain comprises a S228P, F234A, and/or a L235A mutation. In some embodiments, the human IgG4 derived Fc domain does not comprise a S228P, F234A, and/or a L235A mutation.

In some embodiments, the multispecific construct comprises an Fc domain with one or more amino acid substitutions which improve ADCC. In some embodiments, the one or more substitutions are at positions 298, 333, and/or 334 of the Fc region (EU numbering of residues).

In some embodiments, alterations are made in the Fc domain that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the multispecific construct comprises a variant Fc domain comprising one or more amino acid substitutions which alters half-life and/or changes binding to the neonatal Fe receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fe region to FcRn. Such Fe variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fe region variants.

In some embodiments, the multispecific construct comprises a Fc domain comprising an amino acid sequence having at least 80%, at least 85%, at least 90%, at least 95%, at least 96%, at least 97%, at least 98%, or at least 99% identity with anyone selected from the group consisting of SEQ ID NOs: 46-56. In some embodiments, the multispecific construct comprises a Fc domain comprising an amino acid sequence of anyone selected from the group consisting of SEQ ID NOs: 46-56.

### Cysteine Engineered Antibody Variants and Heterodimers

In some embodiments, it may be desirable to create cysteine engineered antibody moieties, e.g., “thioMAbs,” in which one or more residues of one or more of the antibody moieties in a multispecific construct herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

### Heterodimers

Usually, two identical Fc regions form a homodimer. However, two different Fe regions can form a heterodimer with mutations to the one or two individual chain via, for example, a knob-into-hole (KIH), a disulfide bond (—S—S—), or via hydrophobic interaction, electrostatic interaction, hydrophilic interaction, or increased flexibility.

The term “knob-into-hole” or “KIH” technology as used herein refers to the technology directing the pairing of two polypeptides together in vitro or in vivo by introducing a protuberance (knob) into one polypeptide and a cavity (hole) into the other polypeptide at an interface in which they interact. For example, KIHs have been introduced in the Fc:Fc binding interfaces, CL:CH1 interfaces or VH/VL interfaces of antibodies (see, e.g., US 201 1/0287009, US2007/0178552, WO 96/02701 1, WO 98/050431, Zhu et al, 1997, Protein Science 6:781-788, and WO2012/106587). In some embodiments, KIHs drive the pairing of two different heavy chains together during the manufacture of multispecific antibodies. For example, multispecific antibodies having KIH in their Fc regions can further comprise single variable domains linked to each Fc region, or further comprise different heavy chain variable domains that pair with similar or different light chain variable domains. KIH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

In some embodiments, a knob mutation in an IgG1 constant region is T366W (EU numbering). In some embodiments, a hole mutation in an IgG1 constant region comprises one or more mutations selected from T366S, L368A, and Y407V (EU numbering).

In some embodiments, a knob mutation in an IgG1 constant region is S354C and T366W (EU numbering). In some embodiments, a hole mutation in an IgG1 constant region comprises one or more mutations selected from Y349C, T366S, L368A, and Y407V (EU numbering).

Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al, Science, 229: 81 (1985)); using leucine zippers to produce bi-specific antibodies (see, e.g., Kostelny et al, J. Immunol, 148(5): 1547-1553 (1992)); using “diabody” technology for making bispecific antibody fragments (see, e.g., Hollinger et al, Proc. Natl. Acad. Sci. USA, 90:6444-6448 (1993)); and using single-chain Fv (sFv) dimers (see, e.g. Gruber et al, J.Immunol, 152:5368 (1994)); and preparing trispecific antibodies as described, e.g., in Tutt et al. J. Immunol. 147: 60 (1991).

In some embodiments, the Fc domain provided herein comprises a knob mutation and the pairing Fc domain comprises hole mutation(s), or vice versa.

In some embodiments, the multispecific construct comprises a Fc domain comprising an amino acid sequence of any one of SEQ ID NOs: 285-286 and 288-289.

In some embodiments, the multispecific construct comprises a Fc domain comprising an amino acid sequence of SEQ ID NO: 285 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 285, and a pairing Fc domain comprising an amino acid sequence of SEQ ID NO: 288 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 288.

In some embodiments, the multispecific construct comprises a Fc domain comprising an amino acid sequence of SEQ ID NO: 286, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 286, and a pairing Fc domain comprising an amino acid sequence of SEQ ID NO: 289 or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 289.

In some embodiments, the Fc domain and/or the pairing Fc domain provided herein are IgG Fc domain. In some embodiments, the IgG is IgG1, IgG2, IgG3 or IgG4.

### Anti-4-1BB Antibody Moiety

The anti-4-1BB antibody moieties of the multispecific constructs described in the present application include any antibody moieties that specifically bind to 4-1BB. In some embodiments, the 4-1BB is a human 4-1BB (“h4-1BB”). h4-1BB is a type I transmembrane receptor with four extracellular cysteine-rich domains (“CRDs”, i.e., CRD1, CDR2, CRD3, and CRD4) followed by a short transmembrane domain and a C-terminal cytoplasmic region. CRD2 and CRD3 of h4-1BB interact with the ligand 4-1BBL (Bitra et al. (2018) J Biol Chem. 293(26): 9958-9969. In contrast to other TNFRs, h4-1BB exists as a disulfide-linked dimer, and Dimerization likely occurs through an unpaired cysteine (Cysm¹²¹) found within CRD4 of h4-1BB. In some embodiments, the h4-1BB comprises the sequence set forth in SEQ ID NO: 41 or a variant thereof (e.g., a post translationally modified variant and/or conformation variant). In some embodiments, the anti-4-1BB antibody moiety binds to the CRD3/CRD4 region of 4-1BB.

The anti-4-1BB antibody moiety can be any suitable format known in the art. In some embodiments, the anti-4-1BB antibody moiety is selected from the group consisting of full-length antibody, Fab, Fab′, F(ab′)₂, scFv, and sdAb. In some embodiments, the anti-4-1BB antibody moiety comprises a single-domain antibody that binds to 4-1BB.

### Exemplary Anti-4-1BB Antibody Moieties

In some embodiments, the anti-4-1BB antibody moiety comprises a single domain antibody (sdAb) comprising a sdAb-CDR1, a sdAb-CDR2, and a sdAb-CDR3, respectively comprising the amino acid sequence of a CDR1, a CDR2, and a CDR3 within a single monomeric variable antibody domain having the amino acid sequence set forth in SEQ ID NO: 27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27.

In some embodiments, the anti-4-1BB antibody moiety comprises a single domain antibody (sdAb) comprising a sdAb-CDR1, a sdAb-CDR2, and a sdAb-CDR3, respectively comprising the amino acid sequence of a CDR1, a CDR2, and a CDR3 within a single monomeric variable antibody domain having the amino acid sequence set forth in SEQ ID NO: 27, and the CDR1, a CDR2, and a CDR3 are according to Kabat numbering scheme. In some embodiments, the anti-4-1BB antibody moiety comprises a single domain antibody (sdAb) comprising a sdAb-CDR1, a sdAb-CDR2, and a sdAb-CDR3, respectively comprising the amino acid sequence of a CDR1, a CDR2, and a CDR3 within a single monomeric variable antibody domain having the amino acid sequence set forth in SEQ ID NO: 27, and the CDR1, a CDR2, and a CDR3 are according to IMGT numbering scheme. In some embodiments, the anti-4-1BB antibody moiety comprises a single domain antibody (sdAb) comprising a sdAb-CDR1, a sdAb-CDR2, and a sdAb-CDR3, respectively comprising the amino acid sequence of a CDR1, a CDR2, and a CDR3 within a single monomeric variable antibody domain having the amino acid sequence set forth in SEQ ID NO: 27, and the CDR1, a CDR2, and a CDR3 are according to Kabat numbering scheme.

In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

In some embodiments, the anti-4-1BB antibody moiety comprises a single domain antibody (sdAb) comprising: a) a sdAb-CDR1 comprising an amino acid sequence of SEQ ID NO: 24, or a variant thereof comprising up to about 3 (such as any of about 1, 2, 3) amino acid substitutions in the sdAb-CDR1; b) a sdAb-CDR2 comprising an amino acid sequence of SEQ ID NO: 25, or a variant thereof comprising up to about 3 (such as any of about 1, 2, 3) amino acid substitutions in the sdAb-CDR2; and c) a sdAb-CDR3 comprising an amino acid sequence of SEQ ID NO: 26, or a variant thereof comprising up to about 3 (such as any of about 1, 2, 3) amino acid substitutions in the sdAb-CDR3.

In some embodiments, the anti-4-1BB antibody moiety comprises a single domain antibody (sdAb) comprising a) a sdAb-CDR1 comprising an amino acid sequence of SEQ ID NO: 24, a sdAb-CDR2 comprising an amino acid sequence of SEQ ID NO: 25, and a sdAb-CDR3 comprising an amino acid sequence of SEQ ID NO: 26.

In some embodiments, the anti-4-1BB antibody moiety comprises a single domain antibody (sdAb) comprising the amino acid sequence of SEQ ID NOs: 27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27.

### Tumor Antigens

The tumor antigen that the first moiety (such as the first antibody moiety) specifically binds to can be any suitable tumor antigen known in the art. In some embodiments, the tumor antigen is selected from the group consisting of MUC16, ENPP3, ROR1, SLC7A11, DLL3, B7H4, EPHA2, CD318 and CLDN6.

In some embodiments, the tumor antigen is selected from the group comprising HER2, Nectin-4, 5T4, GPC3, MSLN, FAP, CLDN18.2, PD-L1, PD-L2, ILT-4, B7-H3, CS1, CD19, CD2, CD4, CD5, CD7, CD8, CD20, CD22, CD25, CD28, CD30, CD33, CD38, CD44V6, CD47, CD52, CD56, CD57, CD58, CD79b, CD81, CD123, CD133, CD151, CD171, CD276, CLL1, BCMA, VEGFR-2, GPC3, PMSA, CEACAM6, c-Met, ErbB3, HER3, ErbB4/HER-4, IGF1R, GD2, O-acetyl GD2, O-acetyl GD3, GHRHR, GHR, Flt1, KDR, Flt4, Flt3, CEA, BTLA, TGFBR1, TGFBR2, TGFBR1, IL6R, gp130, Lewis, TNFR1, TNFR2, PD1, PSCA, HVEM, PSMA, RANK, TNFRSF4, TWEAK-R, LTPR, LIFRP, LRP5, MUC1, PTCH1, WT-1, Robo1, Frizzled, Notch-1-4, APRIL, MAGE3, Folate receptor α, Folate receptor β, GPC2, CD70, BAFF-R, and TROP-2.

In some embodiments, the tumor antigen is mucin 16 (MUC16). MUC16 is a member of the mucin family of glycoproteins and is also known as mucin CA125. MUC16 has been shown to play a role in tumorigenesis and tumor proliferation. MUC16 is thought to participate in cell-cell interactions that enable metastasis through binding of mesothelin. MUC16 may also play a role in promoting cell motility and invasion through its C-terminal domain. Exemplary protein sequences of MUC16 can be found, for example, at UniProtKB Q8WX17.

In some embodiments, the first antibody moiety binds to MUC16. Any anti-MUC16 antibody whose binding to MUC16 triggers the second antibody moiety to activate 4-1BB may be used in the present invention. Suitable anti-MUC16 antibodies include, but are not limited to, those described in Bast et al. (J. Clin. Invest. 1981; 68(5):1331-1337), Nustad et al. (Tumour Biol, 1996; 17(4):196-219), Lloyd et al. (Lloyd et al., Int. J. Cancer, 1997; 71(5):842-850), Marcos-Silava et al. (Glycobiology, 2015; 25(11:1172-1182), Chen et al. (Cancer Res., 2007; 67(10):4924-4932), Aithal et al. (PLoS One, 2018; 13(4):e01293907), Gipson et al. (Glycobiology, 2017; 27(1):920-926), Davies et al. (nt. J. Biochem. Cell Biolo., 2007; 39(1):1943-1954), WO 2002/092836, WO 2020/102555, WO 2020/227538, WO 2016/149368, U.S. Pat. Pub. 2021/0309758, U.S. Pat. Pub. 2020/0317810, U.S. Pat. No. 10,941,208, WO 2007/001851, U.S. Pat. No. 7,078,188, WO 2007/001851, WO 2019/213747, and WO 2008/141044, which are hereby incorporated by reference in their entirety. In some embodiments, the anti-MUC16 antibody is selected from the group comprising Mab-AR-9.6 (Quest PharmaTech), Oregovamab (Quest PharmaTech), RG-7458 (Genentech), RG-7882 (Genentech), EDO-772P (Mundipharma EDO GmbH), Abagovomab, NAV-005 (Navrogen). In some embodiments, the anti-MUC16 antibody binds to the N-terminal tandem repeat region of MUC16. In some embodiments, the anti-MUC16 antibody binds to the MUC16 carboxy-terminal region. In some embodiments, the anti-MUC16 antibody binds to the juxtamembrande domain, the cytoplasmic tail, or a region C-terminal to the mucin repeat domain.

In some embodiments, the tumor antigen is ENPP3 (ectonucleotide pyrophosphatase/phosphodiesterase family member3). ENPP3 is also known as NPP3, PDNP3, CD203c, and PD-IBETA. ENPP3 is an ectoenzyme, a class of transmembrane protein that is involved in the hydrolysis of extracellular nucleotides and has been found to be expressed in several cancers and cancerous cells, including neoplastic mast cells, acute basophilic leukemia, colon cancer, renal cell carcinoma, hepatocellular carcinoma, and neoplastic cell bile duct cells. Exemplary protein sequences of ENPP3 can be found, for example, at UniProtKB 014638.

In some embodiments, the first antibody moiety binds to ENPP3. Any anti-ENPP3 antibody whose binding to ENPP3 triggers the second antibody moiety to activate 4-1BB may be used in the present invention. Suitable anti-ENPP3 antibodies include, but are not limited to, those described in Donate et al, (Clin Cancer Res (2016) 22 (8): 1989-1999), U.S. Pat. No. 7,427,399, U.S. Pub. No. 2010/0099111, U.S. Pat. No. 8,562,989, U.S. Pub. No. 2016/0176977, and U.S. Pub. No. 2019/0092874, which are hereby incorporated by reference in their entirety.

In some embodiments, the tumor antigen is tyrosine-protein kinase transmembrane receptor (ROR1). ROR1 is also known as NTRKR1. ROR1 is thought to play a role in tumor cell survival, proliferation, migration, and chemotaxis, and has been to be highly expressed in several cancer types, including chronic lymphocytic leukemia, mantle cell lymphoma, ovarian, breast, prostate, lung, melanoma, and colorectal cancers. Exemplary protein sequences of ROR2 can be found, for example, at UniProtKB Q01973.

In some embodiments, the first antibody moiety binds to ROR1. Any anti-ROR1 antibody whose binding to ROR1 triggers the second antibody moiety to activate 4-1BB may be used in the present invention. Suitable anti-ROR1 antibodies include, but are not limited to, those described in U.S. Pat. Nos. 9,758,591, 10,618,959, WO 2010/124188, WO 2016/187220, WO 2012/045085, WO 2017/072361, WO 2019/008377, WO 2019/005636, WO 2014/031174, and WO 2017/127664 which are hereby incorporated by reference in their entirety. In some embodiments, the first antibody moiety is cirmtuzumab.

In some embodiments, the tumor antigen is SLC7A11. SLC7A11 is a cysteine/glutamate transporter also known as CCBR1, xCT, and solute carrier family 7 member 11. Overexpression of SLC7A11 is thought to promote tumor growth, in part through suppressing ferroptosis. Exemplary protein sequences of SLC7A11 can be found, for example, at UniProtKB Q9UPY5.

In some embodiments, the first antibody moiety binds to SLC7A11. Any anti-SLC7A11 antibody whose binding to SLC7A11 triggers the second antibody moiety to activate 4-1BB may be used in the present invention. Suitable anti-SLC7A11 antibodies include, but are not limited to those described in WO 2018/204278 and WO 2020/227640, which are hereby incorporated by reference in their entirety.

In some embodiments, the tumor antigen is Delta-like 3 (DLL3). DLL3 is a member of the delta protein ligand family that functions as a Notch ligand. High DLL3 expression has been observed in several cancer types, especially in neuroendocrine-related tumors, and has been investigated as a potential target for several cancer types including small cell lung cancer, non-small cell lung cancer, and large cell neuroendocrine carcinoma. High expression of DLL3 is part Exemplary protein sequences of DLL3 can be found, for example, at UniProtKB Q9NYJ7.

In some embodiments, the first antibody moiety binds to DLL3. Any anti-DLL3 antibody whose binding to DLL3 triggers the second antibody moiety to activate 4-1BB may be used in the present invention. Suitable anti-DLL3 antibodies include, but are not limited to, those described in Sauders et al. (Sci Transl Med., 2015; 7(302): 302ra136), WO 2017/031458, WO 2019/217145, WO 2013/126746, WO 2015/127407, WO 2011/093097, and WO 2021/007371, which are hereby incorporated by reference in their entirety. In some embodiments the anti-DLL3 antibody is rovalpituzumab.

In some embodiments, the tumor antigen is B7H4, also known as V-set domain-containing T-cell activation inhibitor 1 (VTCN1) and B7x. B7H4 belongs to the immunoglobulin superfamily and is involved in regulating T-cell proliferation and expansion. B7H4 is highly expressed in several cancers, including ovarian, renal cell cancer, pancreatic cancer, hepatocellular carcinoma, gastric cancer, lung cancer, glioma, breast, prostate cancer, urothelial cancer, cervical cancer and melanoma, and this increased expression is thought to help tumors evade the immune system. Exemplary protein sequences of B7H4 can be found, for example, at UniProtKB Q7Z7D3.

In some embodiments, the first antibody moiety binds to B7H4. Any anti-B7H4 antibody whose binding to B7H4 triggers the second antibody moiety to activate 4-1BB may be used in the present invention. Suitable anti-B7H4 antibodies include, but are not limited to, those described in WO 2019/040780, U.S. Pat. No. 9,562,099, WO 2012/145568, WO 2013/067492, and WO 2014/100483 which are hereby incorporated by reference in their entirety. In some embodiments, the antibody is Alsevalimab (FP150).

In some embodiments, the tumor antigen is EPH receptor A2 (EPHA2). EPHA2 is also known as ECK, CPTA, ARCC2 CTPP1, and CTRCT6. EPHA2 is a member of the ephrin receptor subfamily of the protein tyrosine kinase family. EPHA2 is thought to play a role in tumor growh, invasion, metastatic progression, and drug resistance, and has been reported as overexpressed in several cancer types, including prostate, lung, esophageal, colorectal, cervical, ovarian, breast, and skin cancer. Exemplary protein sequences of EPHA2 can be found, for example, at UniProtKB P29317.

In some embodiments, the first antibody moiety binds to EPHA2. Any anti-EPHA2 antibody whose binding to EPHA2 triggers the second antibody moiety to activate 4-1BB may be used in the present invention. Suitable anti-EPHA2 antibodies include, but are not limited to, those described in Kinch et al. (Cancer Res. 2002; 62(10):2840-2847), Coffman et al. (Cancer Res. 2003; 63(22):7907-7912), Goldgur et al. (Growth Factors. 2014; 32(6):214-222), Sakamoto et al. (Anticancer Res. 2018; 38(6):3273-3282), Bruckheimer et al. (Neoplasia. 2009, 11(6):509-517), Hasegawa et al. (Cancer Biol. Ther., 2016; 17(11): 1158-1167), U.S. Pat. Nos. 7,101,976, 7,776,327, WO 2004/014292, U.S. Pat. Pub. 2007/0086943, U.S. Pat. Pub. 2010/0298545, WO 2016/081601, U.S. Pat. No. 7,659,374, WO 2006/023403, U.S. Pat. No. 10,406,225, and U.S. Pat. Pub. 2016/0031987, which are hereby incorporated by reference in their entirety.

In some embodiments, the tumor antigen is CD318. CD318 is also known as CUB domain-containing protein 1 (CDCP1), SIMA135, and TRASK. CD318 is a transmembrane glycoprotein with an extracellular domain containing two CUB domains. Phosphorylation of CD318 has been observed in many cancer types, including pre-invasive cancers, invasive cancers, and tumor metastases. Exemplary protein sequences of CD318 can be found, for example, at UniProtKB Q9H5V8.

In some embodiments, the first antibody moiety binds to CD318. Any anti-CD318 antibody whose binding to CD318 triggers the second antibody moiety to activate 4-1BB may be used in the present invention. Suitable anti-CD318 antibodies include, but are not limited to, those described in WO 2011/023389, WO 2018/112334, WO 2019/084319, WO 2021/132427, and WO 2011/023390, which are hereby incorporated by reference in their entirety.

In some embodiments, the tumor antigen is Claudin 6 (CLDN6). Claudin-6 (CLDN6) is a member of the claudin family and serves as a tight junction molecule, which plays a vital role in cell-to-cell adhesion in epithelial or endothelial cell sheets. It encodes the tetraspan membrane protein, with the size of 220 amino acids and molecular mass of 23,292 Da. CLDN6 has been identified to be the origination of cell adhesion signaling taking part in the regulation of nuclear receptor activity through targeting molecules of the nuclear receptor superfamily and managing their gene expression (Sugimoto et al. (2019). “Cell adhesion signals regulate the nuclear receptor activity.” Proc. Natl. Acad. Sci. U.S.A. 116, 24600-24609). CLDN6 appears to be significantly upregulated in 20 types of human cancers (Zhang et al. (2021) Front. Cell. Dev. Biol. 9: 726656). In some embodiments, the CLDN6 is a human CLDN6 (“hCLDN6”). In some embodiments, the hCLDN6 comprises an amino acid sequence set forth in SEQ ID NO: 40 or a variant thereof (e.g., a post translationally modified variant and/or conformation variant).

In some embodiments, the first antibody moiety binds to CLDN6. Any anti-CLDN6 antibody whose binding to CLDN6 triggers the second antibody moiety to activate 4-1BB may be used in the present invention. Suitable anti-CLDN6 antibodies include, but are not limited to, any one of the anti-CLDN6 antibodies recited herein, U.S. Pat. No. 9,274,119, WO 2012/156018, WO 2019/056023, and U.S. Pat. No. 10,053,511, which are hereby incorporated by reference in their entirety.

In some embodiments, the tumor antigen is programmed death-ligand 1 (PD-L1), also known as CD274 and B7-H1. PD-L1 is a transmembrane protein that plays a role in suppressing the adaptive immune system. PD-L1 binds to PD-1, which is expressed on T cells, B cells, and myeloid cells. Overexpression of PD-L1 on tumor cells is thought to help cancers evade the immune system. Exemplary protein sequences of PD-L1 can be found, for example, at UniProtKB Q9NZ17.

In some embodiments, the first antibody moiety binds to PD-L1. Any anti-PD-L1 antibody whose binding to PD-L1 triggers the second antibody moiety to activate 4-1BB may be used in the present invention. Suitable anti-PD-L1 antibodies include, but are not limited to, Atezolizumab, Avelumab, Durvalumab, atezolizumab (e.g., Tecentriq®), avelumab (e.g., Bavencio®), and durvalumab (e.g., IMFINZI™)

### Multispecific Constructs

In one aspect, provided herein is a multispecific construct comprising: a first moiety (such as first antibody moiety) that specifically binds to a tumor antigen; and a second antibody moiety that specifically binds to 4-1BB, wherein binding of the first antibody moiety with the tumor antigen triggers the second antibody moiety to activate 4-1BB. In some embodiments, the second antibody moiety specifically binds to CRD 3/4 region of 4-1BB.

In some embodiments, provided herein is a multispecific construct comprising a first antibody moiety that specifically binds to a tumor antigen; and a second antibody moiety that specifically binds to 4-1BB, wherein binding of the first antibody moiety with the tumor antigen triggers the second antibody moiety to activate 4-1BB. In some embodiments, the activation of 4-1BB by the second antibody moiety is enhanced by at least about any one of 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen, including any range in between these values. In some embodiments, without binding to the tumor antigen, the multispecific construct does activate 4-1BB signaling. In some embodiments, without binding to the tumor antigen, the second moiety does not activate 4-1BB signaling.

In some embodiments of the multispecific construct of the present application, the second antibody moiety is a sdAb. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27. In some embodiments, the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27, and the CDR1, a CDR2, and a CDR3 are according to Kabat numbering scheme.

In some embodiments, the sdAb comprises a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3.

In some embodiments, the sdAb comprises the amino acid sequence of SEQ ID NO:27, or a variant thereof having at least about 80% (including for example at least about any of 80%, 85%, 87%, 89%, 90%, 91%, 92%, 93%, 94%, 95%, 96%, 97%, 98%, or 99%, or more than 99%) sequence identity to the sequence set forth in SEQ ID NO: 27. In some embodiments, the affinity of such sdAb for 4-1BB (e.g., human 4-1BB) is comparable (e.g., the same as) to that that of an sdAb comprising SEQ ID NO: 27.

In some embodiments, the tumor antigen that the first antibody moiety specifically binds to can be any suitable tumor antigen known in the art. In some embodiments, the tumor antigen is selected from the group consisting of MUC16, ENPP3, ROR1, SLC7A11, DLL3, B7H4, EPHA2, CD318 and CLDN6.

In some embodiments, the present application provides multispecific constructs that bind to both MUC16 and 4-1BB. In some embodiments, the multispecific construct described herein is a bispecific antibody comprising an anti-MUC16 antibody moiety and an anti-4-1BB antibody moiety. Anti-MUC16 antibody moieties and anti-4-1BB antibody moieties can be any of those described herein.

In some embodiments, the present application provides multispecific constructs that bind to both ENPP3 and 4-1BB. In some embodiments, the multispecific construct described herein is a bispecific antibody comprising an anti-ENPP3 antibody moiety and an anti-4-1BB antibody moiety. Anti-ENPP3 antibody moieties and anti-4-1BB antibody moieties can be any of those described herein.

In some embodiments, the present application provides multispecific constructs that bind to both ROR1 and 4-1BB. In some embodiments, the multispecific construct described herein is a bispecific antibody comprising an anti-ROR1 antibody moiety and an anti-4-1BB antibody moiety. Anti-ROR1 antibody moieties and anti-4-1BB antibody moieties can be any of those described herein.

In some embodiments, the present application provides multispecific constructs that bind to both SLC7A11 and 4-1BB. In some embodiments, the multispecific construct described herein is a bispecific antibody comprising an anti-SLC7A11 antibody moiety and an anti-4-1BB antibody moiety. Anti-SLC7A11 antibody moieties and anti-4-1BB antibody moieties can be any of those described herein.

In some embodiments, the present application provides multispecific constructs that bind to both DLL3 and 4-1BB. In some embodiments, the multispecific construct described herein is a bispecific antibody comprising an anti-DLL3 antibody moiety and an anti-4-1BB antibody moiety. Anti-DLL3 antibody moieties and anti-4-1BB antibody moieties can be any of those described herein.

In some embodiments, the present application provides multispecific constructs that bind to both B7H4 and 4-1BB. In some embodiments, the multispecific construct described herein is a bispecific antibody comprising an anti-B7H4 antibody moiety and an anti-4-1BB antibody moiety. Anti-B7H4 antibody moieties and anti-4-1BB antibody moieties can be any of those described herein.

In some embodiments, the present application provides multispecific constructs that bind to both EPHA2 and 4-1BB. In some embodiments, the multispecific construct described herein is a bispecific antibody comprising an anti-EPHA2 antibody moiety and an anti-4-1BB antibody moiety. Anti-EPHA2 antibody moieties and anti-4-1BB antibody moieties can be any of those described herein.

In some embodiments, the present application provides multispecific constructs that bind to both CD318 and 4-1BB. In some embodiments, the multispecific construct described herein is a bispecific antibody comprising an anti-CD318 antibody moiety and an anti-4-1BB antibody moiety. Anti-CD318 antibody moieties and anti-4-1BB antibody moieties can be any of those described herein.

In some embodiments, the present application provides multispecific constructs that bind to both CLDN6 and 4-1BB. In some embodiments, the multispecific construct described herein is a bispecific antibody comprising an anti-CLDN6 antibody moiety and an anti-4-1BB antibody moiety. Anti-CLDN6 antibody moieties and anti-4-1BB antibody moieties can be any of those described herein.

In some embodiments, multispecific construct is bi-paratopic and comprises a third antibody moiety that specifically binds to the same tumor antigen as the first antibody moiety but with a different epitope.

In some embodiments, the anti-4-1BB antibody moiety comprises a sdAb comprising: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the anti-4-1BB antibody moiety comprises an sdAb comprising the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27.

In some embodiments, the multispecific construct described herein is a multispecific construct (e.g., a bispecific or bi-paratopic or trispecific antibody) comprising an anti-tumor antigen antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-tumor antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a third antibody moiety that specifically binds to the same tumor antigen as the first antibody moiety but with a different epitope. In some embodiments, the first antibody moiety (and/or the third antibody moiety) is selected from the group consisting of an scFv, a Fab, a half-antibody, a single chain half-antibody and a full length antibody. The format of the third antibody moiety can be the same or different from the first antibody moiety. For example, both the first and third antibody moiety can be full-length antibody format. For another example, the first antibody moiety is a full-length antibody and the third antibody moiety is a scFv, or vice versa. For yet another example, the first antibody moiety is a half-antibody and the third antibody moiety is a single chain half-antibody, or vice versa.

In some embodiments, the first antibody moiety (and/or the third antibody moiety) binds to the tumor antigen with an affinity of about 10⁻⁷ M to about 10⁻¹³ M. In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with the tumor antigen triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-MUC16 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-MUC16 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-MUC16 moiety that specifically binds to the same tumor antigen as the first anti-MUC16 moiety but with a different epitope. In some embodiments, the first antibody moiety (and/or the third antibody moiety) is selected from the group consisting of an scFv, a Fab, a half-antibody, a single chain half-antibody and a full length antibody. The format of the second anti-MUC16 moiety can be the same or different from the first anti-MUC16 moiety. For example, both the first and third anti-MUC16 moiety can be full-length antibody format. For another example, the first anti-MUC16 moiety is a full-length antibody and the second anti-MUC16 moiety is a scFv, or vice versa. For yet another example, the first anti-MUC16 moiety is a half-antibody and the second anti-MUC16 moiety is a single chain half-antibody, or vice versa.

In some embodiments, the first antibody moiety (and/or the third antibody moiety) binds to MUC16 with an affinity of about 10⁻⁷ M to about 10⁻¹³ M. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with MUC16 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ENPP3 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-ENPP3 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-ENPP3 moiety that specifically binds to the same tumor antigen as the first anti-ENPP3 moiety but with a different epitope. The format of the second anti-ENPP3 moiety can be the same or different from the first anti-ENPP3 moiety. For example, both the first and third anti-ENPP3 moiety can be full-length antibody format. For another example, the first anti-ENPP3 moiety is a full-length antibody and the second anti-ENPP3 moiety is a scFv, or vice versa. For yet another example, the first anti-ENPP3 moiety is a half-antibody and the second anti-ENPP3 moiety is a single chain half-antibody, or vice versa.

In some embodiments, the first antibody moiety (and/or the third antibody moiety) binds to ENPP3 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full-length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with ENPP3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ROR1 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-ROR1 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-ROR1 moiety that specifically binds to the same tumor antigen as the first anti-ROR1 moiety but with a different epitope. The format of the second anti-ROR1 moiety can be the same or different from the first anti-ROR1 moiety. For example, both the first and third anti-ROR1 moiety can be full-length antibody format. For another example, the first anti-ROR1 moiety is a full-length antibody and the second anti-ROR1 moiety is a scFv, or vice versa. For yet another example, the first anti-ROR1 moiety is a half-antibody and the second anti-ROR1 moiety is a single chain half-antibody, or vice versa.

In some embodiments, the first antibody moiety (and/or the third antibody moiety) binds to ROR1 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with ROR1 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-SLC7A11 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-SLC7A11 antigen antibody moiety and an anti-4-1BB antibody moiety, and the anti-4-1BB antibody moiety comprises a sdAb. In some embodiments, multispecific constructs is bi-paratopic and thus comprises a second anti-SLC7A11 moiety that specifically binds to the same tumor antigen as the first anti-SLC7A11 moiety but with a different epitope. The format of the second anti-SLC7A11 moiety can be the same or different from the first anti-SLC7A11 moiety. For example, both the first and third anti-SLC7A11 moiety can be full-length antibody format. For another example, the first anti-SLC7A11 moiety is a full-length antibody and the second anti-SLC7A11 moiety is a scFv, or vice versa. For yet another example, the first anti-SLC7A11 moiety is a half-antibody and the second anti-SLC7A11 moiety is a single chain half-antibody, or vice versa.

In some embodiments, the first antibody moiety is not an sdAb. In some embodiments, the first antibody moiety (and/or the third antibody moiety) binds to SLC7A11 with an affinity of about 10⁻⁷ M to about 10⁻³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with SLC7A11 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-DLL3 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-DLL3 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-DLL3 moiety that specifically binds to the same tumor antigen as the first anti-DLL3 moiety but with a different epitope. The format of the second anti-DLL3 moiety can be the same or different from the first anti-DLL3 moiety. For example, both the first and third anti-DLL3 moiety can be full-length antibody format. For another example, the first anti-DLL3 moiety is a full-length antibody and the second anti-DLL3 moiety is a scFv, or vice versa. For yet another example, the first anti-DLL3 moiety is a half-antibody and the second anti-DLL3 moiety is a single chain half-antibody, or vice versa.

In some embodiments, the first antibody moiety (and/or the third antibody moiety) binds to DLL3 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with DLL3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-B7H4 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-B7H4 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-B7H4 moiety that specifically binds to the same tumor antigen as the first anti-B7H4 moiety but with a different epitope. The format of the second anti-B7H4 moiety can be the same or different from the first anti-B7H4 moiety. For example, both the first and third anti-B7H4 moiety can be full-length antibody format. For another example, the first anti-B7H4 moiety is a full-length antibody and the second anti-B7H4 moiety is a scFv, or vice versa. For yet another example, the first anti-B7H4 moiety is a half-antibody and the second anti-B7H4 moiety is a single chain half-antibody, or vice versa.

In some embodiments, the first antibody moiety (and/or the third antibody moiety) binds to B7H4 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full-length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with B7H4 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-EPHA2 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-EPHA2 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-EPHA2 moiety that specifically binds to the same tumor antigen as the first anti-EPHA2 moiety but with a different epitope. The format of the second anti-EPHA2 moiety can be the same or different from the first anti-EPHA2 moiety. For example, both the first and third anti-EPHA2 moiety can be full-length antibody format. For another example, the first anti-EPHA2 moiety is a full-length antibody and the second anti-EPHA2 moiety is a scFv, or vice versa. For yet another example, the first anti-EPHA2 moiety is a half-antibody and the second anti-EPHA2 moiety is a single chain half-antibody, or vice versa.

In some embodiments, the first antibody moiety (and/or the third antibody moiety) binds to EPHA2 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with EPHA2 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-CD318 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-CD318 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-CD318 moiety that specifically binds to the same tumor antigen as the first anti-CD318 moiety but with a different epitope. The format of the second anti-CD318 moiety can be the same or different from the first anti-CD318 moiety. For example, both the first and third anti-CD318 moiety can be full-length antibody format. For another example, the first anti-CD318 moiety is a full-length antibody and the second anti-CD318 moiety is a scFv, or vice versa. For yet another example, the first anti-CD318 moiety is a half-antibody and the second anti-CD318 moiety is a single chain half-antibody, or vice versa.

In some embodiments, the first antibody moiety (and/or the third antibody moiety) binds to CD318 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with CD318 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-CLDN6 antibody moiety and an anti-4-1BB antibody moiety, and the anti-4-1BB antibody moiety specifically binds to CRD 3/4 region of 4-1BB. In some embodiments, the multispecific construct described herein is a multispecific construct (e.g., a bispecific antibody) comprising an anti-CLDN6 antigen antibody moiety and an anti-4-1BB antibody moiety, and the anti-4-1BB antibody moiety comprises a sdAb. In some embodiments, the first antibody moiety is not an sdAb. In some embodiments, multispecific constructs is bi-paratopic and thus comprises a second anti-CLDN6 moiety that specifically binds to the same tumor antigen as the first anti-CLDN6 moiety but with a different epitope. The format of the second anti-CLDN6 moiety can be the same or different from the first anti-CLDN6 moiety. For example, both the first and third anti-CLDN6 moiety can be full-length antibody format. For another example, the first anti-CLDN6 moiety is a full-length antibody and the second anti-CLDN6 moiety is a scFv, or vice versa. For yet another example, the first anti-CLDN6 moiety is a half-antibody and the second anti-CLDN6 moiety is a single chain half-antibody, or vice versa.

In some embodiments, the first antibody moiety (and/or the third antibody moiety) binds to CLDN6 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1 or IgG3 domain). In some embodiments, the first antibody moiety (and/or the third antibody moiety) and the second antibody moiety are connected via an Fc domain (such as an Fc domain derived from IgG1 or IgG3 domain). In some embodiments, the binding of the first binding moiety (and/or the third antibody moiety) with CLDN6 triggers the second antibody moiety to activate 4-1BB, for example an activation that is enhanced by at least 10 folds, or an activation that results an in increase in IFNγ, IL-2, or NFκB production or activity.

In some embodiments, multispecific constructs is bi-paratopic and thus comprises a second anti-CLDN6 moiety that specifically binds to the same tumor antigen as the first anti-CLDN6 moiety but with a different epitope. The format of the second anti-CLDN6 moiety can be the same or different from the first anti-CLDN6 moiety. For example, both the first and third anti-CLDN6 moiety can be full-length antibody format. For another example, the first anti-CLDN6 moiety is a full-length antibody and the second anti-CLDN6 moiety is a scFv, or vice versa. For yet another example, the first anti-CLDN6 moiety is a half-antibody and the second anti-CLDN6 moiety is a single chain half-antibody, or vice versa.

In some embodiments, the multispecific construct described herein is a multispecific construct (e.g., a bispecific or bi-paratopic or tri-specific antibody) comprising an anti-tumor antigen antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-tumor antigen moiety that specifically binds to the same tumor antigen as the first anti-tumor antigen moiety but with a different epitope. The format of the second anti-tumor antigen moiety can be the same or different from the first anti-tumor antigen moiety. For example, both the first and third anti-tumor antigen moiety can be full-length antibody format. For another example, the first anti-tumor antigen moiety is a full-length antibody and the second anti-tumor antigen moiety is a scFv, or vice versa. For yet another example, the first anti-tumor antigen moiety is a half-antibody and the second anti-TUMOR ANTIGEN moiety is a single chain half-antibody, or vice versa.

In some embodiments, the first antibody moiety (and/or the third antibody moiety) binds to the tumor antigen with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full length antibody. In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third binding moiety) with the tumor antigen triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-MUC16 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-MUC16 moiety that specifically binds to the same tumor antigen as the first anti-MUC16 moiety but with a different epitope. The format of the second anti-MUC16 moiety can be the same or different from the first anti-MUC16 moiety. For example, both the first and third anti-MUC16 moiety can be full-length antibody format. For another example, the first anti-MUC16 moiety is a full-length antibody and the second anti-MUC16 moiety is a scFv, or vice versa. For yet another example, the first anti-MUC16 moiety is a half-antibody and the second anti-MUC16 moiety is a single chain half-antibody, or vice versa.

In some embodiments, the first antibody moiety (and/or the third antibody moiety) binds to MUC16 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with MUC16 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-ENPP3 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-ENPP3 moiety that specifically binds to the same tumor antigen as the first anti-ENPP3 moiety but with a different epitope. The format of the second anti-ENPP3 moiety can be the same or different from the first anti-ENPP3 moiety. For example, both the first and third anti-ENPP3 moiety can be full-length antibody format. For another example, the first anti-ENPP3 moiety is a full-length antibody and the second anti-ENPP3 moiety is a scFv, or vice versa. For yet another example, the first anti-ENPP3 moiety is a half-antibody and the second anti-ENPP3 moiety is a single chain half-antibody, or vice versa.

In some embodiments, the first antibody moiety (and/or the third antibody moiety) binds to ENPP3 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with ENPP3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-ROR1 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-ROR1 moiety that specifically binds to the same tumor antigen as the first anti-ROR1 moiety but with a different epitope. The format of the second anti-ROR1 moiety can be the same or different from the first anti-ROR1 moiety. For example, both the first and third anti-ROR1 moiety can be full-length antibody format. For another example, the first anti-ROR1 moiety is a full-length antibody and the second anti-ROR1 moiety is a scFv, or vice versa. For yet another example, the first anti-ROR1 moiety is a half-antibody and the second anti-ROR1 moiety is a single chain half-antibody, or vice versa.

In some embodiments, the first antibody moiety (and/or the third antibody moiety) binds to ROR1 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with ROR1 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-SLC7A11 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-SLC7A11 moiety that specifically binds to the same tumor antigen as the first anti-SLC7A11 moiety but with a different epitope. The format of the second anti-SLC7A11 moiety can be the same or different from the first anti-SLC7A11 moiety. For example, both the first and third anti-SLC7A11 moiety can be full-length antibody format. For another example, the first anti-SLC7A11 moiety is a full-length antibody and the second anti-SLC7A11 moiety is a scFv, or vice versa. For yet another example, the first anti-SLC7A11 moiety is a half-antibody and the second anti-SLC7A11 moiety is a single chain half-antibody, or vice versa.

In some embodiments, the first antibody moiety (and/or the third antibody moiety) binds to SLC7A11 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with SLC7A11 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-DLL3 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-DLL3 moiety that specifically binds to the same tumor antigen as the first anti-DLL3 moiety but with a different epitope. The format of the second anti-DLL3 moiety can be the same or different from the first anti-DLL3 moiety. For example, both the first and third anti-DLL3 moiety can be full-length antibody format. For another example, the first anti-DLL3 moiety is a full-length antibody and the second anti-DLL3 moiety is a scFv, or vice versa. For yet another example, the first anti-DLL3 moiety is a half-antibody and the second anti-DLL3 moiety is a single chain half-antibody, or vice versa.

In some embodiments, the first antibody moiety (and/or the third antibody moiety) binds to DLL3 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with DLL3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-B7H4 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-B7H4 moiety that specifically binds to the same tumor antigen as the first anti-B7H4 moiety but with a different epitope. The format of the second anti-B7H4 moiety can be the same or different from the first anti-B7H4 moiety. For example, both the first and third anti-B7H4 moiety can be full-length antibody format. For another example, the first anti-B7H4 moiety is a full-length antibody and the second anti-B7H4 moiety is a scFv, or vice versa. For yet another example, the first anti-B7H4 moiety is a half-antibody and the second anti-B7H4 moiety is a single chain half-antibody, or vice versa.

In some embodiments, the first antibody moiety (and/or the third antibody moiety) binds to B7H4 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with B7H4 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-EPHA2 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-EPHA2 moiety that specifically binds to the same tumor antigen as the first anti-EPHA2 moiety but with a different epitope. The format of the second anti-EPHA2 moiety can be the same or different from the first anti-EPHA2 moiety. For example, both the first and third anti-EPHA2 moiety can be full-length antibody format. For another example, the first anti-EPHA2 moiety is a full-length antibody and the second anti-EPHA2 moiety is a scFv, or vice versa. For yet another example, the first anti-EPHA2 moiety is a half-antibody and the second anti-EPHA2 moiety is a single chain half-antibody, or vice versa.

In some embodiments, the first antibody moiety (and/or the third antibody moiety) binds to EPHA2 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with EPHA2 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-CD318 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-CD318 moiety that specifically binds to the same tumor antigen as the first anti-CD318 moiety but with a different epitope. The format of the second anti-CD318 moiety can be the same or different from the first anti-CD318 moiety. For example, both the first and third anti-CD318 moiety can be full-length antibody format. For another example, the first anti-CD318 moiety is a full-length antibody and the second anti-CD318 moiety is a scFv, or vice versa. For yet another example, the first anti-CD318 moiety is a half-antibody and the second anti-CD318 moiety is a single chain half-antibody, or vice versa.

In some embodiments, the first antibody moiety (and/or the third antibody moiety) binds to CD318 with an affinity of about 10⁻⁷ M to about 10⁻¹ M. In some embodiments, the first antibody moiety (and/or the third antibody moiety) is selected from the group consisting of an scFv, a Fab, a half-antibody, a single chain half-antibody and a full length antibody. In some embodiments, the multispecific construct comprises an Fc domain (such as an Fc domain derived from IgG1, IgG2, IgG3 or IgG4). In some embodiments, the first antibody moiety and the second antibody moiety are connected via an Fc domain (such as an Fc domain derived from IgG1, IgG2, IgG3 or IgG4). In some embodiments, the binding of the first binding moiety (and/or the third antibody moiety) with CD318 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-CLDN6 antibody moiety and an anti-4-1BB antibody moiety, wherein the anti-4-1BB antibody moiety comprises a sdAb and the sdAb comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NOs: 27, or a variant thereof having at least about 80% sequence identity (e.g., at least about any one of 80%, 85%, 87%, 89%, 90%, 91%, 92%, 93%, 94%, 95%, 96%, 97%, 98%, 99%, or more than 99% sequence identity, including any range in between these values) to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-CLDN6 moiety that specifically binds to the same tumor antigen as the first anti-CLDN6 moiety but with a different epitope. The format of the second anti-CLDN6 moiety can be the same or different from the first anti-CLDN6 moiety. For example, both the first and third anti-CLDN6 moiety can be full-length antibody format. For another example, the first anti-CLDN6 moiety is a full-length antibody and the second anti-CLDN6 moiety is a scFv, or vice versa. For yet another example, the first anti-CLDN6 moiety is a half-antibody and the second anti-CLDN6 moiety is a single chain half-antibody, or vice versa.

In some embodiments, the first antibody moiety (and/or the third antibody moiety) binds to CLDN6 with an affinity of about 10⁻⁷ M to about 10⁻¹³ M. In some embodiments, the first antibody moiety (and/or the third antibody moiety) is selected from the group consisting of an scFv, a Fab, a half-antibody, a single chain half-antibody and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with CLDN6 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or tri-specific antibody) comprising an anti-tumor antigen antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-tumor antigen moiety that specifically binds to the same tumor antigen as the first anti-tumor antigen moiety but with a different epitope. The format of the second anti-tumor antigen moiety can be the same or different from the first anti-tumor antigen moiety. For example, both the first and third anti-tumor antigen moiety can be full-length antibody format. For another example, the first anti-tumor antigen moiety is a full-length antibody and the second anti-tumor antigen moiety is a scFv, or vice versa. For yet another example, the first anti-tumor antigen moiety is a half-antibody and the second anti-tumor antigen moiety is a single chain half-antibody, or vice versa.

In some embodiments, the first antibody moiety (and/or the third antibody moiety) binds to the tumor antigen with an affinity of about 10⁻⁷ M to about 10⁻¹³ M. In some embodiments, the first antibody moiety (and/or the third antibody moiety) is selected from the group consisting of an scFv, a Fab, a half-antibody and a full length antibody. In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with the tumor antigen triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-MUC16 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-MUC16 moiety that specifically binds to the same tumor antigen as the first anti-MUC16 moiety but with a different epitope. The format of the second anti-MUC16 moiety can be the same or different from the first anti-MUC16 moiety. For example, both the first and third anti-MUC16 moiety can be full-length antibody format. For another example, the first anti-MUC16 moiety is a full-length antibody and the second anti-MUC16 moiety is a scFv, or vice versa. For yet another example, the first anti-MUC16 moiety is a half-antibody and the second anti-MUC16 moiety is a single chain half-antibody, or vice versa.

In some embodiments, the first antibody moiety (and/or the third antibody moiety) binds to MUC16 with an affinity of about 10⁻⁷ M to about 10⁻¹ M. In some embodiments, the first antibody moiety is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with MUC16 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ENPP3 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-ENPP3 moiety that specifically binds to the same tumor antigen as the first anti-ENPP3 moiety but with a different epitope. The format of the second anti-ENPP3 moiety can be the same or different from the first anti-ENPP3 moiety. For example, both the first and third anti-ENPP3 moiety can be full-length antibody format. For another example, the first anti-ENPP3 moiety is a full-length antibody and the second anti-ENPP3 moiety is a scFv, or vice versa. For yet another example, the first anti-ENPP3 moiety is a half-antibody and the second anti-ENPP3 moiety is a single chain half-antibody, or vice versa.

In some embodiments, the first antibody moiety (and/or the third antibody moiety) binds to ENPP3 with an affinity of about 10⁻⁷ M to about 10⁻¹³ M. In some embodiments, the first antibody moiety is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as ones an Fc domain derived from IgG1, IgG2, IgG3 or IgG4). In some embodiments, the binding of the first binding moiety (and/or the third antibody moiety) with ENPP3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ROR1 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-ROR1 moiety that specifically binds to the same tumor antigen as the first anti-ROR1 moiety but with a different epitope. The format of the second anti-ROR1 moiety can be the same or different from the first anti-ROR1 moiety. For example, both the first and third anti-ROR1 moiety can be full-length antibody format. For another example, the first anti-ROR1 moiety is a full-length antibody and the second anti-ROR1 moiety is a scFv, or vice versa. For yet another example, the first anti-ROR1 moiety is a half-antibody and the second anti-ROR1 moiety is a single chain half-antibody, or vice versa.

In some embodiments, the first antibody moiety (and/or the third antibody moiety) binds to ROR1 with an affinity of about 10⁻⁷ M to about 10⁻¹³ M. In some embodiments, the first antibody moiety is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety (and/or the third antibody moiety) with ROR1 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-SLC7A11 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-SLC7A11 moiety that specifically binds to the same tumor antigen as the first anti-SLC7A11 moiety but with a different epitope. The format of the second anti-SLC7A11 moiety can be the same or different from the first anti-SLC7A11 moiety. For example, both the first and third anti-SLC7A11 moiety can be full-length antibody format. For another example, the first anti-SLC7A11 moiety is a full-length antibody and the second anti-SLC7A11 moiety is a scFv, or vice versa. For yet another example, the first anti-SLC7A11 moiety is a half-antibody and the second anti-SLC7A11 moiety is a single chain half-antibody, or vice versa.

In some embodiments, the first antibody moiety (and/or the third antibody moiety) binds to SLC7A11 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with SLC7A11 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-DLL3 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-DLL3 moiety that specifically binds to the same tumor antigen as the first anti-DLL3 moiety but with a different epitope. The format of the second anti-DLL3 moiety can be the same or different from the first anti-DLL3 moiety. For example, both the first and third anti-DLL3 moiety can be full-length antibody format. For another example, the first anti-DLL3 moiety is a full-length antibody and the second anti-DLL3 moiety is a scFv, or vice versa. For yet another example, the first anti-DLL3 moiety is a half-antibody and the second anti-DLL3 moiety is a single chain half-antibody, or vice versa.

In some embodiments, the first antibody moiety (and/or the third antibody moiety) binds to DLL3 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with DLL3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-B7H4 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-B7H4 moiety that specifically binds to the same tumor antigen as the first anti-B7H4 moiety but with a different epitope. The format of the second anti-B7H4 moiety can be the same or different from the first anti-B7H4 moiety. For example, both the first and third anti-B7H4 moiety can be full-length antibody format. For another example, the first anti-B7H4 moiety is a full-length antibody and the second anti-B7H4 moiety is a scFv, or vice versa. For yet another example, the first anti-B7H4 moiety is a half-antibody and the second anti-B7H4 moiety is a single chain half-antibody, or vice versa.

In some embodiments, the first antibody moiety (and/or the third antibody moiety) binds to B7H4 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with B7H4 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-EPHA2 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-EPHA2 moiety that specifically binds to the same tumor antigen as the first anti-EPHA2 moiety but with a different epitope. The format of the second anti-EPHA2 moiety can be the same or different from the first anti-EPHA2 moiety. For example, both the first and third anti-EPHA2 moiety can be full-length antibody format. For another example, the first anti-EPHA2 moiety is a full-length antibody and the second anti-EPHA2 moiety is a scFv, or vice versa. For yet another example, the first anti-EPHA2 moiety is a half-antibody and the second anti-CD318 moiety is a single chain half-antibody, or vice versa.

In some embodiments, the first antibody moiety (and/or the third antibody moiety) binds to EPHA2 with an affinity of about 10⁻⁷ M to about 10⁻¹ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with EPHA2 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-CD318 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-CD318 moiety that specifically binds to the same tumor antigen as the first anti-CD318 moiety but with a different epitope. The format of the second anti-CD318 moiety can be the same or different from the first anti-CD318 moiety. For example, both the first and third anti-CD318 moiety can be full-length antibody format. For another example, the first anti-CD318 moiety is a full-length antibody and the second anti-CD318 moiety is a scFv, or vice versa. For yet another example, the first anti-CD318 moiety is a half-antibody and the second anti-CD318 moiety is a single chain half-antibody, or vice versa.

In some embodiments, the first antibody moiety (and/or the third antibody moiety) binds to CD318 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with CD318 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-CLDN6 antibody moiety and an anti-4-1BB antibody moiety, wherein the anti-4-1BB antibody moiety comprises a sdAb and the sdAb comprises: a sdAb-CDR1 comprising an amino acid sequence of SEQ ID NO: 24, or a variant thereof comprising up to about 3 (such as any of about 1, 2, 3) amino acid substitutions in the sdAb-CDR1; a sdAb-CDR2 comprising an amino acid sequence of SEQ ID NO: 25, or a variant thereof comprising up to about 3 (such as any of about 1, 2, 3) amino acid substitutions in the sdAb-CDR2; and a sdAb-CDR3 comprising an amino acid sequence of SEQ ID NO: 26, or a variant thereof comprising up to about 3 (such as any of about 1, 2, 3) amino acid substitutions in the sdAb-CDR3. In some embodiments, the first antibody moiety is not an sdAb. In some embodiments, multispecific constructs is bi-paratopic and thus comprises a second anti-CLDN6 moiety that specifically binds to the same tumor antigen as the first anti-CLDN6 moiety but with a different epitope. The format of the second anti-CLDN6 moiety can be the same or different from the first anti-CLDN6 moiety. For example, both the first and third anti-CLDN6 moiety can be full-length antibody format. For another example, the first anti-CLDN6 moiety is a full-length antibody and the second anti-CLDN6 moiety is a scFv, or vice versa. For yet another example, the first anti-CLDN6 moiety is a half-antibody and the second anti-CLDN6 moiety is a single chain half-antibody, or vice versa.

In some embodiments, the first antibody moiety (and/or the third antibody moiety) binds to CLDN6 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with CLDN6 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-tumor antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-tumor antigen moiety that specifically binds to the same tumor antigen as the first anti-tumor antigen moiety but with a different epitope. The format of the second anti-tumor antigen moiety can be the same or different from the first anti-tumor antigen moiety. For example, both the first and third anti-tumor antigen moiety can be full-length antibody format. For another example, the first anti-tumor antigen moiety is a full-length antibody and the second anti-tumor antigen moiety is a scFv, or vice versa. For yet another example, the first anti-tumor antigen moiety is a half-antibody and the second anti-tumor antigen moiety is a single chain half-antibody, or vice versa.

In some embodiments, the first antibody moiety (and/or the third antibody moiety) binds to the tumor antigen with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the tumor antigen triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-MUC16 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-MUC16 moiety that specifically binds to the same tumor antigen as the first anti-MUC16 moiety but with a different epitope. The format of the second anti-MUC16 moiety can be the same or different from the first anti-MUC16 moiety. For example, both the first and third anti-MUC16 moiety can be full-length antibody format. For another example, the first anti-MUC16 moiety is a full-length antibody and the second anti-MUC16 moiety is a scFv, or vice versa. For yet another example, the first anti-MUC16 moiety is a half-antibody and the second anti-MUC16 moiety is a single chain half-antibody, or vice versa.

In some embodiments, the first antibody moiety (and/or the third antibody moiety) binds to MUC16 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the MUC16 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ENPP3 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-ENPP3 moiety that specifically binds to the same tumor antigen as the first anti-ENPP3 moiety but with a different epitope. The format of the second anti-ENPP3 moiety can be the same or different from the first anti-ENPP3 moiety. For example, both the first and third anti-ENPP3 moiety can be full-length antibody format. For another example, the first anti-ENPP3 moiety is a full-length antibody and the second anti-ENPP3 moiety is a scFv, or vice versa. For yet another example, the first anti-ENPP3 moiety is a half-antibody and the second anti-ENPP3 moiety is a single chain half-antibody, or vice versa.

In some embodiments, the first antibody moiety (and/or the third antibody moiety) binds to ENPP3 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the ENPP3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-ROR1 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-ROR1 moiety that specifically binds to the same tumor antigen as the first anti-ROR1 moiety but with a different epitope. The format of the second anti-ROR1 moiety can be the same or different from the first anti-ROR1 moiety. For example, both the first and third anti-ROR1 moiety can be full-length antibody format. For another example, the first anti-ROR1 moiety is a full-length antibody and the second anti-ROR1 moiety is a scFv, or vice versa. For yet another example, the first anti-ROR1 moiety is a half-antibody and the second anti-ROR1 moiety is a single chain half-antibody, or vice versa.

In some embodiments, the first antibody moiety (and/or the third antibody moiety) binds to ROR1 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the ROR1 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-SLC7A11 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-SLC7A11 moiety that specifically binds to the same tumor antigen as the first anti-SLC7A11 moiety but with a different epitope. The format of the second anti-SLC7A11 moiety can be the same or different from the first anti-SLC7A11 moiety. For example, both the first and third anti-SLC7A11 moiety can be full-length antibody format. For another example, the first anti-SLC7A11 moiety is a full-length antibody and the second anti-SLC7A11 moiety is a scFv, or vice versa. For yet another example, the first anti-SLC7A11 moiety is a half-antibody and the second anti-SLC7A11 moiety is a single chain half-antibody, or vice versa.

In some embodiments, the first antibody moiety (and/or the third antibody moiety) binds to SLC7A11 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the SLC7A11 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-DLL3 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-DLL3 moiety that specifically binds to the same tumor antigen as the first anti-DLL3 moiety but with a different epitope. The format of the second anti-DLL3 moiety can be the same or different from the first anti-DLL3 moiety. For example, both the first and third anti-DLL3 moiety can be full-length antibody format. For another example, the first anti-DLL3 moiety is a full-length antibody and the second anti-DLL3 moiety is a scFv, or vice versa. For yet another example, the first anti-DLL3 moiety is a half-antibody and the second anti-DLL3 moiety is a single chain half-antibody, or vice versa.

In some embodiments, the first antibody moiety (and/or the third antibody moiety) binds to DLL3 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the DLL3 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-B7H4 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-B7H4 moiety that specifically binds to the same tumor antigen as the first anti-B7H4 moiety but with a different epitope. The format of the second anti-B7H4 moiety can be the same or different from the first anti-B7H4 moiety. For example, both the first and third anti-B7H4 moiety can be full-length antibody format. For another example, the first anti-B7H4 moiety is a full-length antibody and the second anti-B7H4 moiety is a scFv, or vice versa. For yet another example, the first anti-B7H4 moiety is a half-antibody and the second anti-B7H4 moiety is a single chain half-antibody, or vice versa.

In some embodiments, the first antibody moiety (and/or the third antibody moiety) binds to B7H4 with an affinity of about 10⁻⁷ M to about 10⁻¹ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the B7H4 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-EPHA2 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-EPHA2 moiety that specifically binds to the same tumor antigen as the first anti-EPHA2 moiety but with a different epitope. The format of the second anti-EPHA2 moiety can be the same or different from the first anti-EPHA2 moiety. For example, both the first and third anti-EPHA2 moiety can be full-length antibody format. For another example, the first anti-EPHA2 moiety is a full-length antibody and the second anti-EPHA2 moiety is a scFv, or vice versa. For yet another example, the first anti-EPHA2 moiety is a half-antibody and the second anti-EPHA2 moiety is a single chain half-antibody, or vice versa.

In some embodiments, the first antibody moiety (and/or the third antibody moiety) binds to EPHA2 with an affinity of about 10⁻⁷ M to about 10⁻¹³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the EPHA2 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific or bi-paratopic or trispecific antibody) comprising an anti-CD318 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-CD318 moiety that specifically binds to the same tumor antigen as the first anti-CD318 moiety but with a different epitope. The format of the second anti-CD318 moiety can be the same or different from the first anti-CD318 moiety. For example, both the first and third anti-CD318 moiety can be full-length antibody format. For another example, the first anti-CD318 moiety is a full-length antibody and the second anti-CD318 moiety is a scFv, or vice versa. For yet another example, the first anti-CD318 moiety is a half-antibody and the second anti-CD318 moiety is a single chain half-antibody, or vice versa.

In some embodiments, the first antibody moiety (and/or the third antibody moiety) binds to CD318 with an affinity of about 10⁻⁷ M to about 10⁻¹ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the CD318 triggers the second antibody moiety to activate 4-1BB, for example an activation that is enhanced by at least 10 folds, or an activation that results an in increase in IFNγ, IL-2, or NFκB production or activity.

In some embodiments, the multispecific construct described herein is a multispecific construct (e.g., a bispecific antibody) comprising an anti-CLDN6 antigen antibody moiety and an anti-4-1BB antibody moiety, wherein the anti-4-1BB antibody moiety comprises a sdAb and the sdAb comprises the amino acid sequence of SEQ ID NO:27, or a variant thereof having at least about 80% (e.g., at least about any one of 80%, 85%, 87%, 89%, 90%, 91%, 92%, 93%, 94%, 95%, 96%, 97%, 98%, 99%, or more than 99% sequence identity, including any range in between these values) sequence identity to SEQ ID NO: 27. In some embodiments, the affinity of such anti-4-1BB antibody moiety for 4-1BB (e.g., human 4-1BB) is comparable (e.g., the same as) to that that of an anti-4-1BB antibody moiety comprising SEQ ID NO: 27. In some embodiments, the first antibody moiety is not an sdAb. In some embodiments, multispecific constructs is bi-paratopic and thus comprises a second anti-CLDN6 moiety that specifically binds to the same tumor antigen as the first anti-CLDN6 moiety but with a different epitope. The format of the second anti-CLDN6 moiety can be the same or different from the first anti-CLDN6 moiety. For example, both the first and third anti-CLDN6 moiety can be full-length antibody format. For another example, the first anti-CLDN6 moiety is a full-length antibody and the second anti-CLDN6 moiety is a scFv, or vice versa. For yet another example, the first anti-CLDN6 moiety is a half-antibody and the second anti-CLDN6 moiety is a single chain half-antibody, or vice versa.

In some embodiments, the first antibody moiety (and/or the third antibody moiety) binds to CLDN6 with an affinity of about 10⁻⁷ M to about 10⁻³ M. In some embodiments, the first antibody moiety (and/or the third antibody moiety) is selected from the group consisting of an scFv, a Fab, and a full length antibody. In some embodiments, the multispecific construct comprises an Fc domain (such as an Fc domain derived from IgG1, IgG2, IgG3 or IgG4). In some embodiments, the first antibody moiety (and/or the third antibody moiety) and the second antibody moiety are connected via an Fc domain (such as an Fc domain derived from IgG1, IgG2, IgG3 or IgG4). In some embodiments, the binding of the first binding moiety with the CLDN6 triggers the second antibody moiety to activate 4-1BB, for example an activation that is enhanced by at least 10 folds, or an activation that results an in increase in IFNγ, IL-2, or NFκB production or activity.

In some embodiments, there is provided a multispecific construct (e.g., bispecific antibody) that comprises an anti-tumor antigen antibody moiety comprising an antibody (e.g., a full-length antibody) that specifically binds to the tumor antigen (e.g., human tumor antigen) and an anti-4-1BB antibody moiety that specifically binds 4-1BB, wherein the anti-4-1BB antibody moiety is fused to the N-terminus of the one or both heavy chains of the anti-tumor antigen antibody (e.g., scFv, Fab, or full-length anti-tumor antigen antibody). In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises, from N to C-terminus: the second antibody moiety (e.g., an sdAb), an optional first linker, an optional Fc domain, an optional second linker, and a heavy chain of the first antibody moiety. In some embodiments, the multispecific construct comprises, from N to C-terminus: the second antibody moiety (e.g., an sdAb), an optional first linker, and a heavy chain of the first antibody moiety (e.g., a full length antibody, such as a full length antibody comprising an Fc domain).

In some embodiments, there is provided a multispecific construct (e.g., bispecific antibody) that comprises an anti-tumor antigen antibody moiety comprising an antibody (e.g., scFv, Fab, or a full-length antibody) that specifically binds to the tumor antigen and an anti-4-1BB antibody moiety that specifically binds 4-1BB, wherein the anti-4-1BB antibody moiety is fused to the C-terminus of the one or both heavy chains of the anti-tumor antigen antibody (e.g., the scFv, Fab, or full-length anti-tumor antigen antibody). In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises, from N to C-terminus: a heavy chain of the first antibody moiety (e.g., a sdAb, scFv, or Fab), an optional first linker, an optional Fc domain, an optional second linker, and the second antibody moiety (e.g., an sdAb). In some embodiments, the multispecific construct comprises, from N to C-terminus: a heavy chain of the first antibody moiety (e.g., a full length antibody, such as a full length antibody comprising an Fc domain), an optional first linker, and the second antibody moiety (e.g., an sdAb).

In some embodiments, there is provided a multispecific construct (e.g., bispecific antibody) that comprises an anti-tumor antigen antibody moiety comprising an antibody (e.g., a scFv, Fab, or full-length antibody) that specifically binds to tumor antigen and an anti-4-1BB antibody moiety that specifically binds 4-1BB, wherein the anti-4-1BB antibody moiety is fused to the N-terminus of the one or both light chains of the anti-tumor antigen antibody (e.g., the scFv, Fab, or full-length anti-tumor antigen antibody). In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises, from N to C-terminus: the second antibody moiety (e.g., an sdAb), an optional first linker, an optional Fc domain, an optional second linker, and a light chain of the first antibody moiety. In some embodiments, the multispecific construct comprises, from N to C-terminus: the second antibody moiety (e.g., an sdAb), an optional first linker, and a light chain of the first antibody moiety (e.g., a full length antibody, such as a full length antibody comprising an Fc domain).

In some embodiments, there is provided a multispecific construct (e.g., bispecific antibody) that comprises an anti-tumor antigen antibody moiety comprising an antibody (e.g., a scFv, Fab, or full-length antibody) that specifically binds to tumor antigen and an anti-4-1BB antibody moiety, wherein the anti-4-1BB antibody moiety is fused to the C-terminus of the one or both light chains of the anti-tumor antigen antibody (e.g., the scFv, Fab, or full-length anti-tumor antigen antibody). In some embodiments, the tumor antigen is selected from the group consisting of MUC16, ENPP3, ROR1, SLC7A11, DLL3, B7H4, EPHA2, CD318 and CLDN6. In some embodiments, the multispecific construct comprises, from N to C-terminus: a light chain of the first antibody moiety (e.g., a sdAb, scFv, or Fab), an optional first linker, an optional Fc domain, an optional second linker, and the second antibody moiety (e.g., an sdAb). In some embodiments, the multispecific construct comprises, from N to C-terminus: a light chain of the first antibody moiety (such as a full length antibody comprising an Fc domain), an optional first linker, and the second antibody moiety (e.g., an sdAb).

In some embodiments, there is provided a multispecific construct (e.g., bispecific antibody) that comprises an anti-tumor antigen antibody moiety comprising an antibody (e.g., a full-length antibody) that specifically binds to tumor antigen (e.g., human tumor antigen) and an anti-4-1BB antibody moiety comprising a single domain antibody that binds to 4-1BB (e.g., human 4-1BB), wherein the single domain antibody is fused to the N-terminus of the one or both heavy chains of the anti-tumor antigen antibody (e.g., scFv, Fab, or full-length anti-CLDN6 antibody). In some embodiments, there is provided a multispecific construct (e.g., bispecific antibody) that comprises an anti-tumor antigen antibody moiety comprising an antibody (e.g., a scFv, Fab, or full-length antibody) that specifically binds to tumor antigen and an anti-4-1BB antibody moiety comprising a single domain antibody that binds to 4-1BB, wherein the single domain antibody is fused to the C-terminus of the one or both heavy chains of the anti-tumor antigen antibody (e.g., the scFv, Fab, or full-length anti-CLDN6 antibody).

In some embodiments, there is provided a multispecific construct (e.g., bispecific antibody) that comprises an anti-tumor antigen antibody moiety comprising an antibody (e.g., a scFv, Fab, or full-length antibody) that specifically binds to tumor antigen and an anti-4-1BB antibody moiety comprising a single domain antibody that binds to 4-1BB, wherein the single domain antibody is fused to the N-terminus of the one or both light chains of the anti-tumor antigen antibody (e.g., the scFv, Fab, or full-length anti-CLDN6 antibody). In some embodiments, the tumor antigen is selected from the group consisting of MUC16, ENPP3, ROR1, SLC7A11, DLL3, B7H4, EPHA2, CD318 and CLDN6. In some embodiments, there is provided a multispecific construct (e.g., bispecific antibody) that comprises an anti-tumor antigen antibody moiety comprising an antibody (e.g., a scFv, Fab, or full-length antibody) that specifically binds to tumor antigen and an anti-4-1BB antibody moiety comprising a single domain antibody that binds to 4-1BB, wherein the single domain antibody is fused to the C-terminus of the one or both light chains of the anti-tumor antigen antibody (e.g., the scFv, Fab, or full-length anti-tumor antigen antibody).

In some embodiments, the anti-4-1BB antibody moiety is fused to the anti-tumor antigen antibody moiety via a linker. In some embodiments, the linker is a peptide linker. In some embodiments, the linker has a length of about four to about fifty amino acids. In some embodiments, the linker is selected from the group consisting of (GS)n, (GGGS)n (SEQ ID NO: 290), (GGGGS)n (SEQ ID NO: 287), and (GSGGS)n (SEQ ID NO: 254). In some embodiments, the tumor antigen is selected from the group consisting of MUC16, ENPP3, ROR1, SLC7A11, DLL3, B7H4, EPHA2, CD318 and CLDN6. In some embodiments, the n is 0-8. In some embodiments, the linker comprises an amino acid sequence of GGGGSGGGGSGGGGS (SEQ ID NO: 23). In some embodiments, the linker comprises an amino acid sequence of GGGGSGGGGSGGGGSGGGGS (SEQ ID NO:284) or GGGGSGGGGSGGGGSGGGGSGGGGS (SEQ ID NO:171).

The multispecific construct of the present disclosure may also include a third antibody moiety which binds the same antigen as one of the other two moieties but at different sites as bi-paratopic. For example, the present disclosure provides a bi-paratopic (tri-specific) construct comprising a first antibody moiety and a third antibody moiety that both binds the same tumor antigen. The first antibody moiety and the third antibody moiety are the same antigen but with a different epitope. In certain embodiments, both the first and third antibody moieties provided herein have a full-length antibody/IgG format, respectively, and the second antibody moiety which is an anti-4-1BB antigen-binding moiety provided herein has a single domain antibody (sdAb)/VHH/nanobody format.

In certain embodiments, the first antibody moiety provided herein has an IgG format and the third antibody moiety provided herein has a scFv format, and the anti-4-1BB antigen-binding moiety provided herein has a single domain antibody (sdAb)/VHH/nanobody format. In certain embodiments, The third antibody moiety is fused to N-terminus of the first antibody moiety, and the second antibody moiety is fused to C-terminus of the first antibody moiety. The first antibody moiety and the third antibody is fused via a linker. The first antibody moiety and the second antibody is fused via a linker.

In certain embodiments, the first antibody moiety provided herein has an full-length antibody/IgG format, the third antibody moiety provided herein has a single chain half-antibody format, and the anti-4-1BB antigen-binding moiety provided herein has a single domain antibody (sdAb)/VHH/nanobody format. In certain embodiments, The second antibody moiety is fused to C-terminus of the first and third antibody moiety, respectively. The second antibody moiety and the third antibody is fused via a linker. The first antibody moiety and the second antibody is fused via a linker. In such case, the first antibody moiety and the third antibody moiety can form a heterodimer via Fe region pairing.

The heterodimer via the Fc region pairing can be achieved by forming a knob-into-hole (KIH), a disulfide bond (—S—S—), or by hydrophobic interaction, electrostatic interaction, hydrophilic interaction, or increased flexibility.

In some embodiments, provided herein is a multispecific construct comprising a first antibody moiety that specifically binds to tumor antigen; and a second antibody moiety that specifically binds to 4-1BB, wherein binding of the first antibody moiety with tumor antigen triggers the second antibody moiety to activate 4-1BB. In some embodiments, the tumor antigen is selected from the group consisting of MUC16, ENPP3, ROR1, SLC7A11, DLL3, B7H4, EPHA2, CD318 and CLDN6. In some embodiments, the activation of 4-1BB by the second antibody moiety is enhanced by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen. In some embodiments, without binding to the antigen, the multispecific construct does not activate 4-1BB signaling. In some embodiments, without binding to the tumor antigen, the second moiety does activate 4-1BB signaling. 4-1BB signaling activation is the expected mechanism for agonist antibodies, such as utomilumab (PF-05082566) and urelumab (BMS-663513). The anti-4-1BB portions of some of the presently disclosed antibodies, however, do not require such an activity. Actually, in some embodiments, it is preferred that the anti-4-1BB portions of the present antibodies are not capable of independently activating 4-1BB in the absence of tumor antigen binding. As the experimental examples demonstrated, interestingly, when the anti-tumor antigen portion binds to tumor antigen on a cell, such binding can trigger 4-1BB signaling activation.

Compared to the known anti-4-1BB agonist antibodies which are commonly associated with dose-limiting on-target hepatotoxicities, the antibodies of the present disclosure are contemplated to be much safer. Because tumor antigen is not expressed in healthy condition, the antibodies of the present disclosure are not expected to trigger cytotoxic immune response as they cannot activate 4-1BB signaling. In a tumor tissue where the tumor antigen is expressed and/or accessible, by contrast, the present antibodies can initiate potent immune response to the tumor cells. Accordingly, unlike those anti-4-1BB antibodies currently being developed clinically which suffer on-target/inherent toxicities, the presently disclosed antibodies can be potent and safe at the same time in treating cancer.

In some embodiments, provided herein is a multispecific construct comprising a first antibody moiety and/or a third antibody moiety that specifically binds to tumor antigen; and a second antibody moiety that specifically binds to 4-1BB, wherein binding of the first antibody moiety and/or the third antibody moiety with tumor antigen triggers the second antibody moiety to activate the immune system. In some embodiments, binding of the first antibody moiety and/or the third antibody moiety with tumor antigen triggers the second antibody moiety to increase the level of one or more cytokines. In some embodiments, the cytokine is IFNγ or IL-2. In some embodiments, binding of the first antibody moiety and/or the third antibody moiety with tumor antigen triggers the second antibody moiety to increase NFκB signaling.

In some embodiments, provided herein is a multispecific construct comprising a first antibody moiety and/or the third antibody moiety that specifically binds to tumor antigen; and a second antibody moiety that specifically binds to 4-1BB, wherein binding of the first antibody moiety and/or the third antibody moiety with tumor antigen triggers the second antibody moiety to activate NFκB signaling.

In some embodiments, activation of NFκB signaling is assessed by measuring changes in the expression of downstream targets of NFκB signaling, such as cytokines, growth factors, adhesion molecules, and/or anti-apoptotic genes. In some embodiments, changes in the expression of downstream targets of NFκB signaling are measured by determining the level of RNA transcript expression of the downstream target of NFκB signaling. Suitable methods of measuring RNA transcript levels in a sample are known in the art, including, for example, by Northern blot analysis, nuclease protection assays, in situ hybridization, PCR analysis (e.g., qPCR, RT-PCR, RT-qPCR, etc.), and next generation sequencing (e.g., RNAseq). In some embodiments, changes in the expression of downstream targets of NFκB signaling are measured by determining the level of protein expression of the downstream target of NFκB signaling. Suitable methods of measuring protein expression in a sample are known in the art, including, for example, immunoassays (e.g., Meso Scale Discovery or MSD assay), immunohistochemistry (IHC), PET imaging, Western blotting, enzyme-linked immunosorbent assays (ELISAs), flow cytometry, and mass spectrometry. In some embodiments, activation of NFκB signaling is assessed by measuring the activation of one or more components of the NFκB signaling cascade, such as by measuring the level of activated IκB kinase and/or IκBα.

In some embodiments, activation of NFκB signaling is assessed by measuring the level of cytoplasmic and/or nuclear NFκB.

In some embodiments, the activation of NFκB signaling by the second antibody moiety is enhanced by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen. In some embodiments, without binding to the antigen, the multispecific construct does not activate NFκB signaling.

In some embodiments, provided herein is a multispecific construct comprising a first antibody moiety and/or the third antibody moiety that specifically binds to tumor antigen; and a second antibody moiety that specifically binds to 4-1BB, wherein binding of the first antibody moiety and/or the third antibody moiety with tumor antigen triggers the activation of the second antibody moiety to increase the level of one or more cytokines.

In some embodiments, the level of the one or more cytokines is measured by determining the level of RNA transcript expression of the one or more cytokines. Suitable methods of measuring RNA transcript levels in a sample are known in the art, including, for example, by Northern blot analysis, nuclease protection assays, in situ hybridization, PCR analysis (e.g., qPCR, RT-PCR, RT-qPCR, etc.), and next generation sequencing (e.g., RNAseq). In some embodiments, the level of transcript expression of the biomarker is measured by RT-PCR, in situ hybridization, and/or RNAseq.

In some embodiments, the level of the one or more cytokines is measured by determining the level of protein expression of the one or more cytokines. Suitable methods of measuring protein expression in a sample are known in the art, including, for example, immunoassays (e.g., Meso Scale Discovery or MSD assay), immunohistochemistry (IHC), PET imaging, Western blotting, enzyme-linked immunosorbent assays (ELISAs), flow cytometry, and mass spectrometry. In some embodiments, the level of protein expression of the biomarker is measured by immunoassay, Western blotting, ELISA, IHC, and/or flow cytometry.

In some embodiments, the level of the one or more cytokines is increased by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety with the tumor antigen. In some embodiments, without binding to the antigen, the multispecific construct does not lead to an increase in the level of the one or more cytokines. In some embodiments, the cytokine is IFNγ and/or IL-12.

In some embodiments, provided herein is a multispecific construct comprising a first antibody moiety and/or the third antibody moiety that specifically binds to tumor antigen; and a second antibody moiety that specifically binds to 4-1BB, wherein binding of the first antibody moiety and/or the third antibody moiety with tumor antigen triggers the activation of the second antibody moiety to increase the level of IFNγ. In some embodiments, the level of IFNγ is increased by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety and/or the third antibody moiety with the tumor antigen. In some embodiments, without binding to the antigen, the multispecific construct does not lead to an increase in the level of IFNγ.

In some embodiments, provided herein is a multispecific construct comprising a first antibody moiety and/or the third antibody moiety that specifically binds to tumor antigen; and a second antibody moiety that specifically binds to 4-1BB, wherein binding of the first antibody moiety and/or the third antibody moiety with tumor antigen triggers the activation of the second antibody moiety to increase the level of IL-2. In some embodiments, the level of IL-2 is increased by at least 2-fold, 3-fold, 4-fold, 5-fold, 6-fold, 7-fold, 8-fold, 9-fold, 10-fold, 20-fold, 50-fold, 100-fold, 200-fold, 500-fold, or 1000-fold after binding of the first antibody moiety and/or the third antibody moiety with the tumor antigen. In some embodiments, without binding to the antigen, the multispecific construct does not lead to an increase in the level of IL-2.

In some embodiments, the level of IFNγ, IL-2 and/or NFκB signaling is measured in one or more (e.g., one or more, two or more, three or more, four or more, etc.) samples obtained from a subject. Any suitable sample in the form of tissues and/or fluids that are known or believed to contain diseased cells and/or a target of interest may be used in the methods described herein, including, for example, sputum, pleural fluid, lymph fluid, bone marrow, blood, plasma, serum, urine, tissue samples (including samples known or expected to contain cancer cells), tumor samples, tumor biopsies, etc. In some embodiments, the sample is a blood sample. In some embodiments, the sample is a serum sample. In some embodiments, the sample is a tumor sample. In some embodiments, the sample is a tumor biopsy. In some embodiments, the sample comprises one or more cancer cells.

Methods of obtaining suitable tissue and/or fluid samples (e.g., methods that are appropriate for obtaining a representative sample from a particular type, location, disease tissue, etc.) are well known to one of ordinary skill in the art, including, for example, by resection, bone marrow biopsy or bone marrow aspiration, endoscopic biopsy or endoscopic aspiration (e.g., cystoscopy, bronchoscopy, colonoscopy, etc.), needle biopsy or needle aspiration (e.g., fine needle aspiration, core needle biopsy, vacuum-assisted biopsy, image-guided biopsy, etc.) skin biopsy (e.g., shave biopsy, punch biopsy, incisional biopsy, excisional biopsy, etc.), various other surgical tissue (e.g., tumor tissue) biopsy and/or excision strategies, and fluid collections (e.g., collecting urine, blood, serum, plasma, sputum, etc.).

### Nucleic Acids

Nucleic acid molecules encoding the multispecific constructs or various antibody moieties described herein are also contemplated. In some embodiments, there is provided a nucleic acid (or a set of nucleic acids) encoding one or more polypeptides of the multispecific constructs or various antibody moieties. In some embodiments, there is provided a nucleic acid (or a set of nucleic acids) encoding a multispecific construct (e.g., an anti-tumor antigen/anti-4-1BB bispecific antibody, bi-paratopic antibody or trispecific antibody), or polypeptide portion thereof.

Also contemplated here are isolated host cell comprising a multispecific construct (e.g., an anti-tumor antigen/anti-4-1BB bispecific antibody, bi-paratopic antibody or trispecific antibody), nucleic acid(s) encoding the polypeptide components of the multispecific construct, or a vector comprising a nucleic acid encoding the polypeptide components of the multispecific construct (e.g., an anti-tumor antigen/anti-4-1BB bispecific antibody, bi-paratopic antibody or trispecific antibody) described herein.

The present application also includes variants to these nucleic acid sequences. For example, the variants include nucleotide sequences that hybridize to the nucleic acid sequences encoding the multispecific construct (e.g., an anti-tumor antigen/anti-4-1BB bispecific antibody, bi-paratopic antibody or trispecific antibody) or various antibody moieties described herein under at least moderately stringent hybridization conditions.

The present application also provides vectors in which a nucleic acid of the present application is inserted.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

### Methods of Treatment

Also provided here are methods of treating a disease or condition in an individual. The methods comprise administering a multispecific construct (e.g., an anti-tumor antigen/anti-4-1BB bispecific antibody) described herein into individuals (e.g., mammals such as humans). In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc.

In some embodiments of the method, the disease or condition is a proliferative disorder. In some embodiments the cell proliferative disorder is cancer. In some embodiments, the cancer is solid tumor, melanoma, renal cancer, ovarian cancer, colorectal cancer, Squamous cell carcinoma of head and neck (SCCHN), non-small cell lung cancer, or non-Hodgkin lymphoma (NHL).

### Compositions, Kits and Articles of Manufacture

Also provided herein are compositions (such as formulations) comprising any one of the multispecific constructs (e.g., an anti-tumor antigen/anti-4-1BB bispecific antibody, bi-paratopic antibody or trispecific antibody) described herein, a nucleic acid encoding any of the multispecific constructs or a portion thereof, a vector comprising the nucleic acid encoding one of the multispecific constructs, or a host cell comprising the nucleic acid or vector.

Suitable formulations of the multispecific construct (e.g., an anti-tumor antigen/anti-4-1BB bispecific antibody, bi-paratopic antibody or trispecific antibody) described herein can be obtained by mixing the multispecific construct having the desired degree of purity with optional pharmaceutically acceptable carriers, excipients or stabilizers (Remington's Pharmaceutical Sciences 16th edition, Osol, A. Ed. (1980)).

Also provided are kits comprising any one of the multispecific constructs (e.g., an anti-tumor antigen/anti-4-1BB bispecific antibody, bi-paratopic antibody or trispecific antibody) described herein. The kits may be useful for any of the methods of treatment described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle).

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. The examples below are intended to be purely exemplary of the application and should therefore not be considered to limit the application in any way. The following examples and detailed description are offered by way of illustration and not by way of limitation.

### Example 1: Generation of CLDN6×4-1BB Bispecific Antibodies

The exemplary CLDN6×4-1BB bispecific antibodies shown in Table 3 below were designed and generated.

TABLE 3
Exemplary CLDN6 x 4-1BB Bispecific Antibodies
1. Anti-CLDN6-1/Anti-4-1BB
CLDN6-1 IgG1 N297A X 4-1BB (CLDN6-1 X 4-1BB NA, 4-1BB sdAb fused to C-terminus of
heavy chain of CLDN-6-1 antibody moiety, with N297A in IgG1 Fc region)

| | | |
|---|---|---|
| Heavy chain | Heavy chain | QVQLQESGPGLVKPSETLSLTCTVSGYSITSGYYWNWIRQPPGKGLEWMGYIS |
| component | of CLDN6-1 | YSGSNNYNPSLKNRVTISRDTSKNQFSLKLSSVTAADTAVYYCARGGYYFDYW |
| (SEQ ID NO: | (SEQ ID NO: | GQGTLVTVSSASTKGPSVFPLAPSSKSTSGGTAALGCLVKDYFPEPVTVSWNS |
| 28) | 10) | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTKPREEQYASTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLEGVAVICT |
| | (SEQ ID NO: | GGGSPSYADSVKGRFTISRDNAKNTLYLQMNSLRAEDTAVYYCAADLLRAGTP |
| | 27) | LSSYEFNYWGQGTLVTVSS |
| Light chain | Light chain of | DIVMTQSPDSLAVSLGERATINCKSSQSLLNTNNQKNYLAWYQQKPGQPPKLL |
| component | CLDN6-1 | IYFASTRESGVPDRFSGSGSGTDFTLTISSLQAEDVAVYFCQQHYNAPRTFGG |
| (SEQ ID NO: | (SEQ ID NO: | GTKVEIKRTVAAPSVFIFPPSDEQLKSGTASVVCLLNNFYPREAKVQWKVDNA |
| 29) | 11) | LQSGNSQESVTEQDSKDSTYSLSSTLTLSKADYEKHKVYACEVTHQGLSSPVT |
| | | KSFNRGEC |

CLDN6-1 IgG1 WT X 4-1BB (CLDN6-1 X 4-1BB WT, 4-1BB sdAb fused to C-terminus of
heavy chain of CLDN-6-1 antibody moiety, with wild type IgG1)

| | | |
|---|---|---|
| Heavy chain | Heavy chain | QVQLQESGPGLVKPSETLSLTCTVSGYSITSGYYWNWIRQPPGKGLEWMGYIS |
| component | of CLDN6-1 | YSGSNNYNPSLKNRVTISRDTSKNQFSLKLSSVTAADTAVYYCARGGYYFDYW |
| (SEQ ID NO: | (SEQ ID NO: | GQGTLVTVSSASTKGPSVFPLAPSSKSTSGGTAALGCLVKDYFPEPVTVSWNS |
| 30) | 9 | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLEGVAVICT |
| | (SEQ ID NO: | GGGSPSYADSVKGRFTISRDNAKNTLYLQMNSLRAEDTAVYYCAADLLRAGTP |
| | 27) | LSSYEFNYWGQGTLVTVSS |
| Light chain | Light chain of | DIVMTQSPDSLAVSLGERATINCKSSQSLLNTNNQKNYLAWYQQKPGQPPKLL |
| component | CLDN6-1 | IYFASTRESGVPDRFSGSGSGTDFTLTISSLQAEDVAVYFCQQHYNAPRTFGG |
| (SEQ ID NO: | (SEQ ID NO: | GTKVEIKRTVAAPSVFIFPPSDEQLKSGTASVVCLLNNFYPREAKVQWKVDNA |
| 31) | 11) | LQSGNSQESVTEQDSKDSTYSLSSTLTLSKADYEKHKVYACEVTHQGLSSPVT |
| | | KSFNRGEC |

4-1BB X CLDN6-1 IgG1 WT (4-1BB X CLDN6-1 WT, 4-1BB sdAb fused to N-terminus of
heavy chain of CLDN-6-1 antibody moiety, with wild type IgG1)

| | | |
|---|---|---|
| Heavy chain | 4-1BB sdAb | QVQLQESGGGLVQPGGSLRLSCAASGYTYSSNCMGWFRQAPGKGLEGVAVICT |
| component | (SEQ ID NO: | GGGSPSYADSVKGRFTISRDNAKNTLYLQMNSLRAEDTAVYYCAADLLRAGTP |
| (SEQ ID NO: | 27) | LSSYEFNYWGQGTLVTVSS |
| 32) | Linker (SEQ | GGGGSGGGGSGGGGS |
| | ID NO: 23) | |
| | Heavy chain | QVQLQESGPGLVKPSETLSLTCTVSGYSITSGYYWNWIRQPPGKGLEWMGYIS |
| | of CLDN6-1 | YSGSNNYNPSLKNRVTISRDTSKNQFSLKLSSVTAADTAVYYCARGGYYFDYW |
| | (SEQ ID NO: | GQGTLVTVSSASTKGPSVFPLAPSSKSTSGGTAALGCLVKDYFPEPVTVSWNS |
| | 9) | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPGK |
| Light chain | Light chain of | DIVMTQSPDSLAVSLGERATINCKSSQSLLNTNNQKNYLAWYQQKPGQPPKLL |
| component | CLDN6 (SEQ | IYFASTRESGVPDRFSGSGSGTDFTLTISSLQAEDVAVYFCQQHYNAPRTFGG |
| (SEQ ID NO: | ID NO: 11) | GTKVEIKRTVAAPSVFIFPPSDEQLKSGTASVVCLLNNFYPREAKVQWKVDNA |
| 33) | | LQSGNSQESVTEQDSKDSTYSLSSTLTLSKADYEKHKVYACEVTHQGLSSPVT |
| | | KSFNRGEC |

4-1BB X CLDN6-1 IgG1 WT (4-1BB X CLDN6-1 L WT, 4-1BB sdAb fused to N-terminus
of light chain of CLDN-6-1 antibody moiety, with wild type IgG1)

| | | |
|---|---|---|
| Heavy chain | Heavy chain | QVQLQESGPGLVKPSETLSLTCTVSGYSITSGYYWNWIRQPPGKGLEWMGYIS |
| component | of CLDN6-1 | YSGSNNYNPSLKNRVTISRDTSKNQFSLKLSSVTAADTAVYYCARGGYYFDYW |
| (SEQ ID NO: | (SEQ ID NO: | GQGTLVTVSSASTKGPSVFPLAPSSKSTSGGTAALGCLVKDYFPEPVTVSWNS |
| 34) | 9) | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKEY |
| | | KCKVSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPGK |
| Light chain | 4-1BB sdAb | QVQLQESGGGLVQPGGSLRLSCAASGYTYSSNCMGWFRQAPGKGLEGVAVICT |
| component | (SEQ ID NO: | GGGSPSYADSVKGRFTISRDNAKNTLYLQMNSLRAEDTAVYYCAADLLRAGTP |
| (SEQ ID NO: | 27) | LSSYEFNYWGQGTLVTVSS |
| 35) | Linker (SEQ | GGGGSGGGGSGGGGS |
| | ID NO: 23) | |
| | Light chain of | DIVMTQSPDSLAVSLGERATINCKSSQSLLNTNNQKNYLAWYQQKPGQPPKLL |
| | CLDN6-1 | IYFASTRESGVPDRFSGSGSGTDFTLTISSLQAEDVAVYFCQQHYNAPRTFGG |
| | (SEQ ID NO: | GTKVEIKRTVAAPSVFIFPPSDEQLKSGTASVVCLLNNFYPREAKVQWKVDNA |
| | 11) | LQSGNSQESVTEQDSKDSTYSLSSTLTLSKADYEKHKVYACEVTHQGLSSPVT |
| | | KSFNRGEC |

CLDN6-3 IgG1 N297A X 4-1BB (CLDN6-3 X 4-1BB NA, 4-1BB sdAb fused to C-terminus
of heavy chain of CLDN-6-3 antibody moiety, with N297A in IgG1 Fc region)

| | | |
|---|---|---|
| Heavy chain | Heavy chain | EVQLVESGGGLVQPGGSLRLSCAASGFTFSIYGMSWVRQAPGKGLEWVATISR |
| component | of CLDN6-3 | DASYTYFPDSVKGRFTISRDNAKNSLYLQMNSLRAEDTAVYYCVRLGDNDRGY |
| (SEQ ID NO: | (SEQ ID NO: | AMDYWGQGTLVTVSSASTKGPSVFPLAPSSKSTSGGTAALGCLVKDYFPEPVT |
| 36) | 21) | VSWNSGALTSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMISRTPEVTC |
| | | VVVDVSHEDPEVKFNWYVDGVEVHNAKTKPREEQYASTYRVVSVLTVLHQDWL |
| | | NGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQG |
| | | NVFSCSVMHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLEGVAVICT |
| | (SEQ ID NO: | GGGSPSYADSVKGRFTISRDNAKNTLYLQMNSLRAEDTAVYYCAADLLRAGTP |
| | 27) | LSSYEFNYWGQGTLVTVSS |
| Light chain | Light chain of | DVVMTQSPLSLPVTLGQPASISCRSSQSVVHVNANTYLEWYQQRPGQSPRLLI |
| component | CLDN6-3 | YKVSNRFPGVPDRFSGSGSGTDFTLKISRVEAEDVGVYYCFQGSHVPRTFGGG |
| (SEQ ID NO: | (SEQ ID NO: | TKVEIKRTVAAPSVFIFPPSDEQLKSGTASVVCLLNNFYPREAKVQWKVDNAL |
| 37) | 22) | QSGNSQESVTEQDSKDSTYSLSSTLTLSKADYEKHKVYACEVTHQGLSSPVTK |
| | | SFNRGEC |

CLDN6-3 IgG1 WT X 4-1BB (CLDN6-3 X 4-1BB WT, 4-1BB sdAb fused to C-terminus of
heavy chain of CLDN-6-3 antibody moiety, with wild type IgG1)

| | | |
|---|---|---|
| Heavy chain | Heavy chain | EVQLVESGGGLVQPGGSLRLSCAASGFTFSIYGMSWVRQAPGKGLEWVATISR |
| component | of CLDN6-3 | DASYTYFPDSVKGRFTISRDNAKNSLYLQMNSLRAEDTAVYYCVRLGDNDRGY |
| (SEQ ID NO: | (SEQ ID NO: | AMDYWGQGTLVTVSSASTKGPSVFPLAPSSKSTSGGTAALGCLVKDYFPEPVT |
| 38) | 20) | VSWNSGALTSGVHTFPAVLQSSGLYSLSSVVTVPSSSLGTQTYICNVNHKPSN |
| | | TKVDKKVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQG |
| | | NVFSCSVMHEALHNHYTQKSLSLSPGK |
| | Linker (SEQ | GGGGSGGGGSGGGGS |
| | ID NO: 23) | |
| | 4-1BB sdAb | QVQLQESGGGLVQPGGSLRLSCAASGYTYSSNCMGWFRQAPGKGLEGVAVICT |
| | (SEQ ID NO: | GGGSPSYADSVKGRFTISRDNAKNTLYLQMNSLRAEDTAVYYCAADLLRAGTP |
| | 27) | LSSYEFNYWGQGTLVTVSS |
| Light chain | Light chain of | DVVMTQSPLSLPVTLGQPASISCRSSQSVVHVNANTYLEWYQQRPGQSPRLLI |
| component | CLDN6-3 | YKVSNRFPGVPDRFSGSGSGTDFTLKISRVEAEDVGVYYCFQGSHVPRTFGGG |
| (SEQ ID NO: | (SEQ ID NO: | TKVEIKRTVAAPSVFIFPPSDEQLKSGTASVVCLLNNFYPREAKVQWKVDNAL |
| 39) | 22) | QSGNSQESVTEQDSKDSTYSLSSTLTLSKADYEKHKVYACEVTHQGLSSPVTK |
| | | SENRGEC |

### Example 2. Antigen Binding Activity of the CLDN6×4-1BB BsAbs

2.1 Binding Affinity of the CLDN6×4-1BB BsAhs with CLDN6

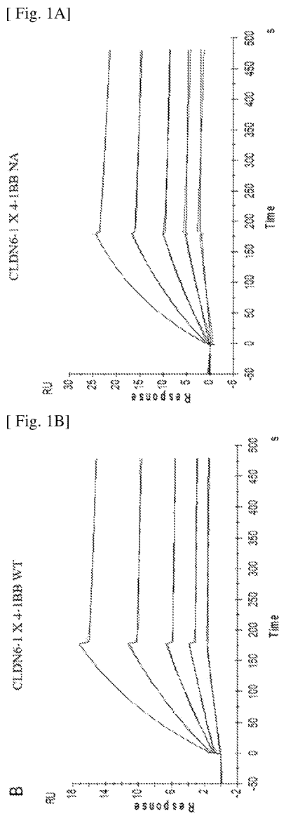

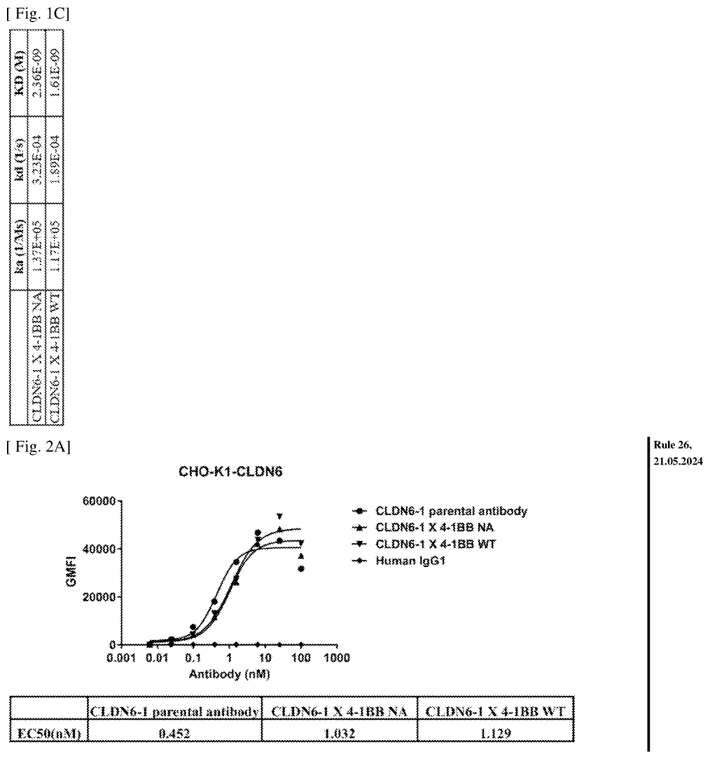

Binding affinity of CLDN6-1×4-1BB NA and CLDN6-1×4-1BB WT (as prepared in Example 1) towards human CLDN6 was measured by surface plasmon resonance (SPR). As shown in FIGS. 1A-1C, CLDN6-1×4-1BB NA and CLDN6-1×4-1BB WT bound to CLDN6 virus-like particles (VLP) with K_{D}s of 1.61×10⁻⁹ M and 2.36×10⁻⁹ M, respectively.

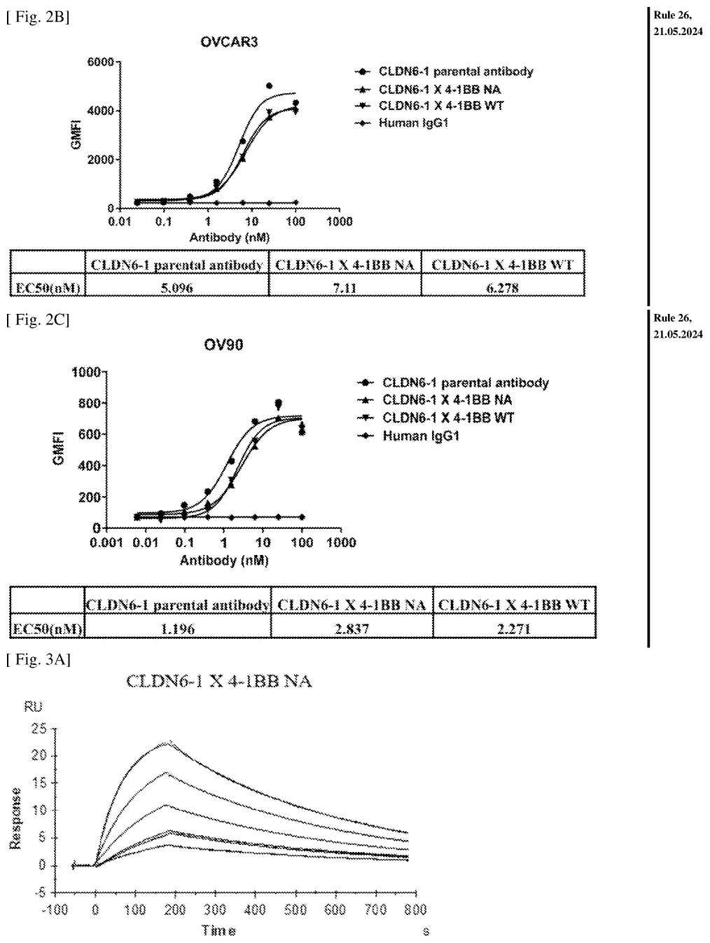

A CHO-K1 cell line stably expressing human CLDN6 (CHO-K1-CLDN6) was prepared to evaluate the binding capability of CLDN6-1×4-1BB NA and CLDN6-1×4-1BB WT towards CLDN6. The parental CLDN6-1 antibody was used as control. Briefly, CHO-K1-CLDN6 cells were incubated with the BsAbs at different concentrations for 30 minutes at 4° C. in FACS buffer. Then, phycoerythrin (PE) conjugated-anti-human IgG antibody was added after washing, and the cells were further incubated at 4° C. for 30 minutes. Mean fluorescence intensity (MFI) of PE was evaluated by FACS. As shown in FIG. 2A, CLDN6-1×4-1BB NA and CLDN6-1×4-1BB WT both bound to CLDN6-expressed cells in a concentration-dependent manner.

OVCAR3 and OV90 are human ovarian cancer cell lines with endogenous CLDN6 expression levels. As shown in FIGS. 2B and 2C, CLDN6-1×4-1BB NA and CLDN6-1×4-1BB WT were both capable of binding to OVCAR3 and OV90. Overall, the binding affinities of the BsAbs comprising an anti-CLDN6-1 antibody moiety for human CLDN6 are comparable to the binding affinity of the parental CLDN6-1 antibody for human CLDN6. The binding signal was well correlated with CLDN6 expression levels on the surfaces of the OVCAR3 and OV90 cells.

2.2 Binding Affinity of CLDN6×4-1BB BsAbs with 4-1BB

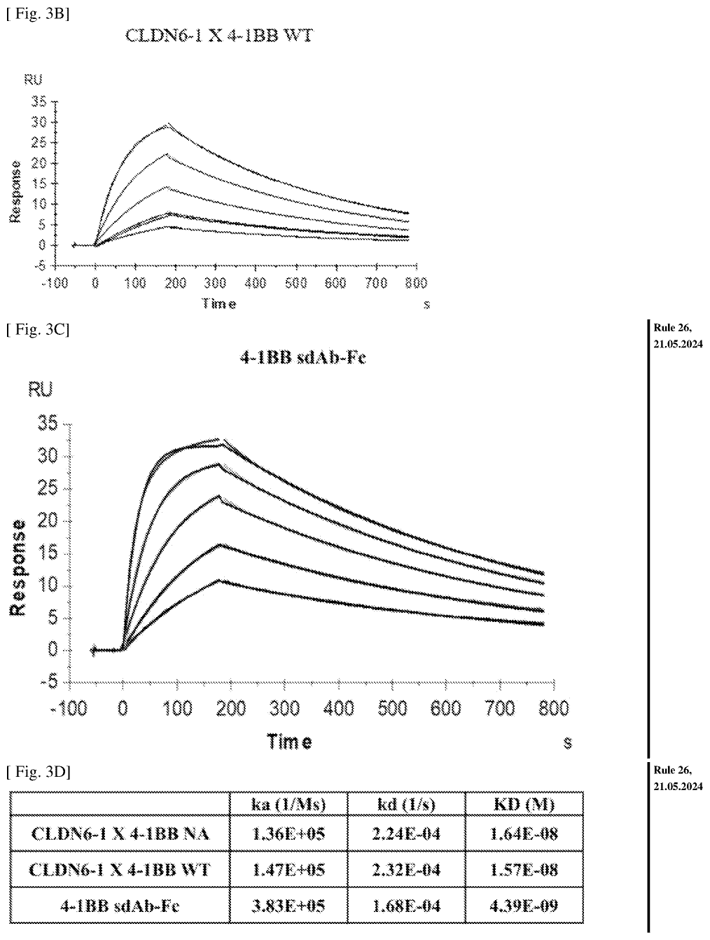

The binding affinity of CLDN6-1×4-1BB NA and CLDN6-1×4-1BB WT towards human 4-1BB was measured by SPR. As shown in FIGS. 3A-3D, CLDN6-1×4-1BB NA and CLDN6-1×4-1BB WT bound to monomeric human 4-1BB with K_{D}s of 1.640×10⁻⁸ M and 1.573×10⁻⁸ M, respectively. The affinity of the parental anti-4-1BB sdAb antibody conjugated with IgG1 Fc fragment (4-1BB sdAb-Fc) for 4-1BB was measured in parallel and was found to have a K_{D} of 4.393×10⁻⁹ M, suggesting that the affinity of the 4-1BB antibody moiety in the BsAbs for 4-1BB is comparable to that of the affinity of 4-1BB sdAb-Fc for 4-1BB.

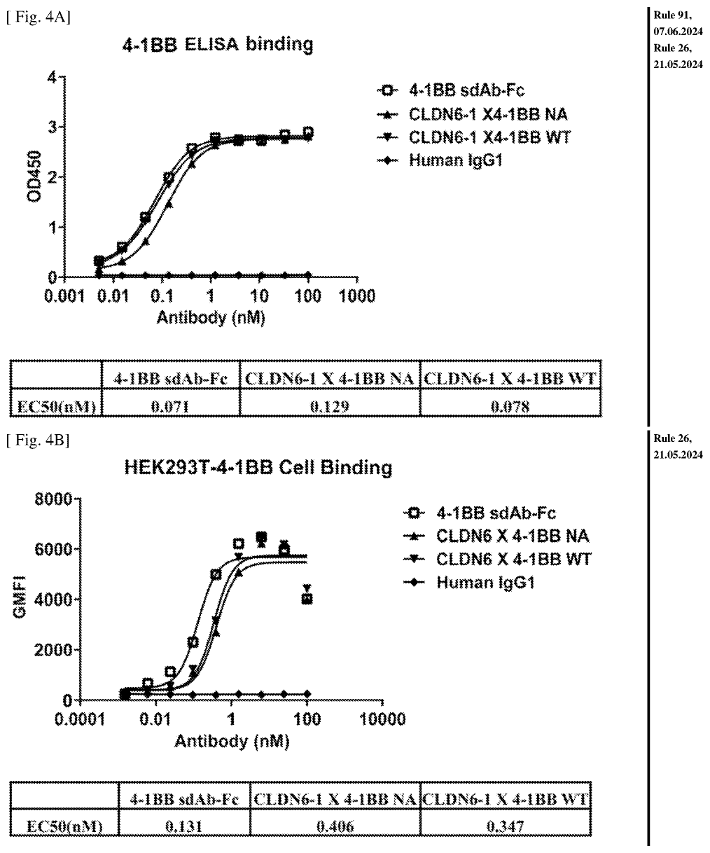

Binding of CLDN6-1×4-1BB NA and CLDN6-1×4-1BB WT with soluble recombinant human 4-1BB was analyzed via ELISA. As shown in FIG. 4A, CLDN6-1×4-1BB NA and CLDN6-1×4-1BB WT both bound to recombinant human 4-1BB in a concentration-dependent manner, with EC50s of 0.129 nM and 0.078 nM, respectively. Such EC50s were comparable with the EC50 of 4-1BB sdAb-Fc. In addition, the binding of CLDN6-1×4-1BB NA and CLDN6-1×4-1BB WT with HEK293 cells expressing 4-1BB was evaluated by FACS. As shown in FIG. 4B, CLDN6-1×4-1BB NA and CLDN6-1×4-1BB WT were both capable of binding to 4-1BB with EC50s of 0.406 nM and 0.347 nM, respectively. Such EC50s were comparable with the EC50 of 4-1BB sdAb-Fc.

### Example 3. Functional Activity of CLDN6-1×4-1BB BsAbs

### 3.1 Cell-Line Based Functional Characterization of CLDN6×4-1BB BsAbs

To test the ability of the CLDN6×4-1BB bispecific antibodies to activate 4-1BB signal, a GloResponse™ NFκB-luc2/4-1BB Jurkat cell line stably expressing 4-1BB and NFκB luciferase reporter was used as effector cells and CLDN6-expressing cells (CHO-K1 CLDN6, OVCAR3 or OV90) were used as target cells. RKO colon carcinoma cells, which do not express CLDN6, expression were used as negative control.

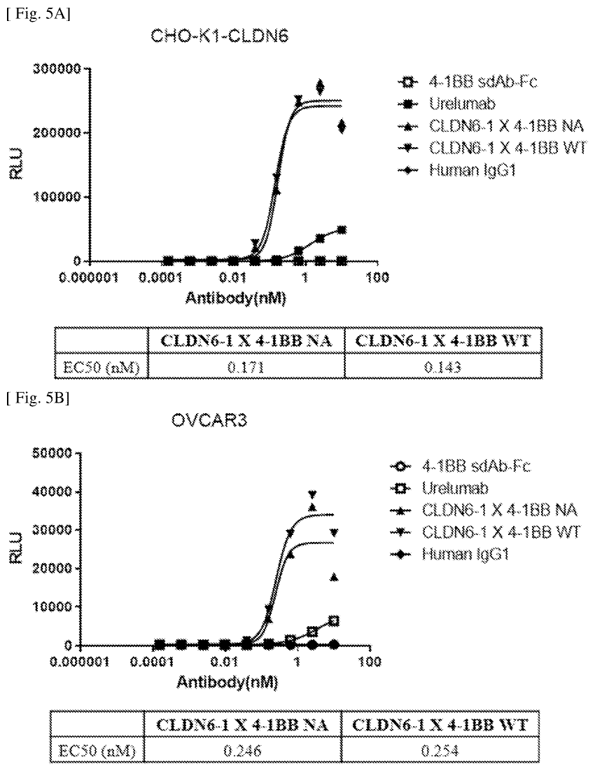

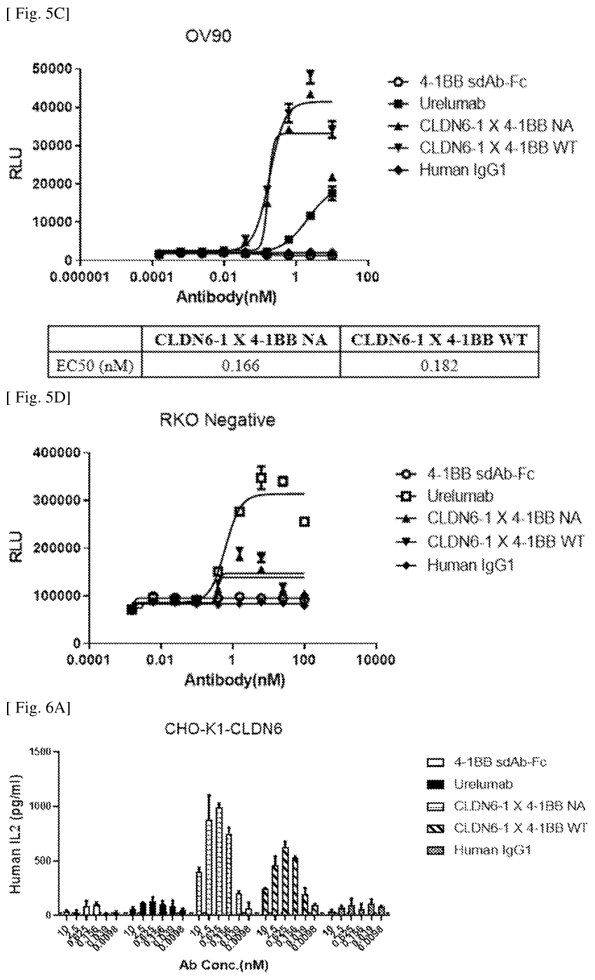

In brief, GloResponse™ NFκB-luc2/4-1BB Jurkat cells (at a density of 5.0×10⁴ cells per well) were mixed with 5.0×10⁴ target cells in a white 96-well plate. Antibodies were serially diluted and added to the plate. Luminescence was measured after 6-hour incubation at 37° C. As shown in FIGS. 5A to 5D, urelumab triggered 4-1BB activation regardless of CLDN6 expression, while 4-1BB sdAb-Fc of the present application had no agonist activity under the same experimental setting, despite its being able to bind to 4-1BB. Similarly, CLDN6-1×4-1BB NA and CLDN6-1×4-1BB WT induced NFκB activity in the presence of all CLDN6-expressing target cells, irrespective of CLDN6 expression levels. In contrast, when RKO cells, which do not express CLDN6, were used as target cells, CLDN6-1×4-1BB NA and CLDN6-1×4-1BB WT resulted in significantly lower 4-1BB activation as compared to urelumab, as shown in FIG. 5D.

### 3.2 Activity of CLDN6×4-1BB BsAbs in Promoting Human Peripheral Blood Mononuclear Cell (PBMC) Immune Response

Pre-activated human PBMCs were cocultured with CLDN6 expressing cell or RKO at an effector-to-target (E:T) ratio of 10:1. Antibodies at different concentrations were added to the mixed culture. After 48 hours, the level of IL-2 or IFNγ in culture medium was measured using homogeneous HTRF assay.

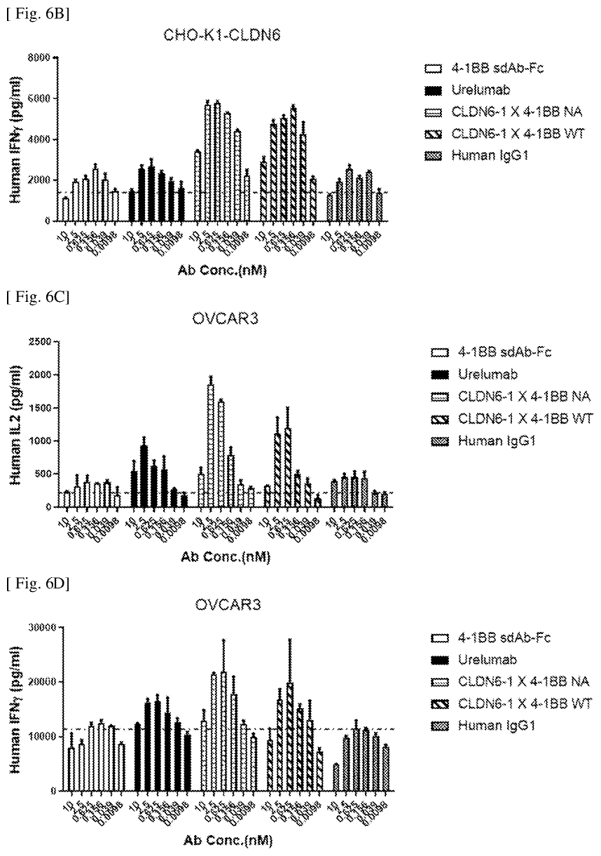

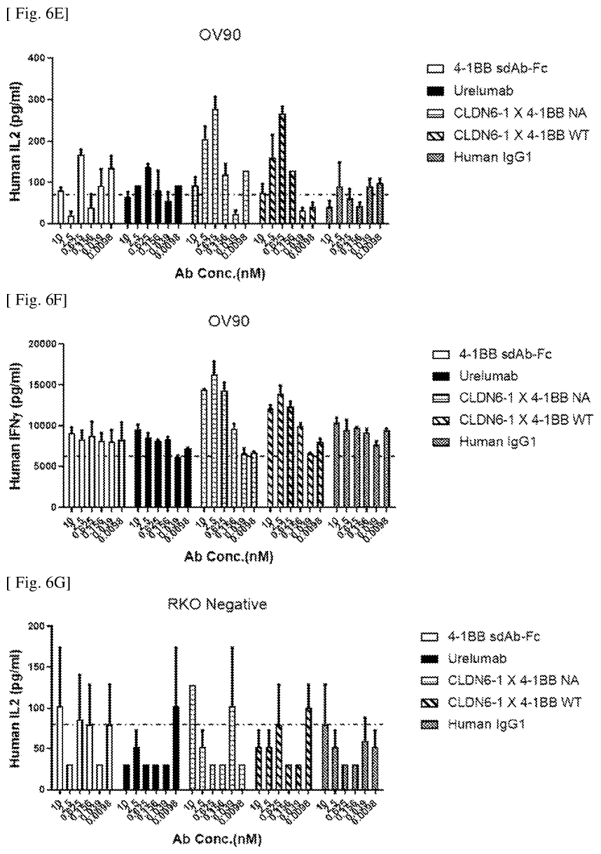

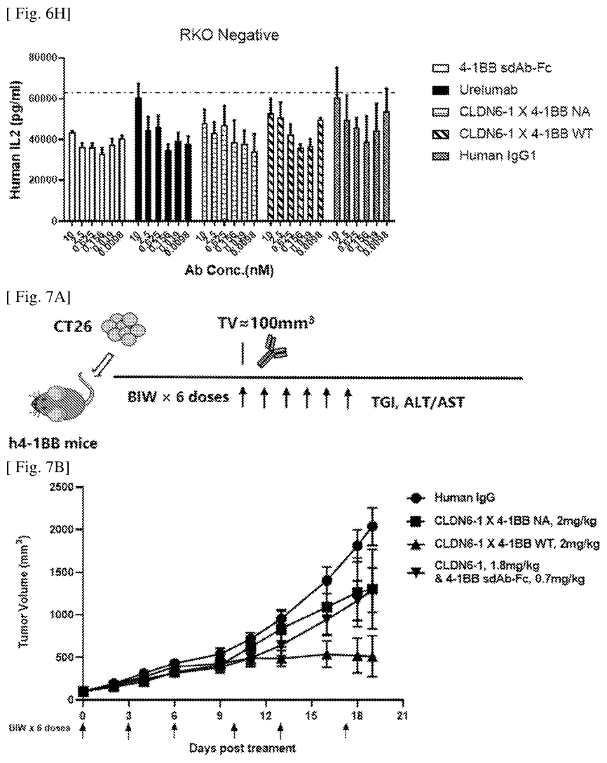

As shown in FIGS. 6A-6F, CLDN6-1×4-1BB NA and CLDN6-1×4-1BB WT stimulated IL-2 and IFNγ production when PBMCs were cocultured with CLDN6-expressing target cells. However, in the presence of RKO that do not express CLDN6, CLDN6-1×4-1BB NA and CLDN6-1×4-1BB WT did not stimulate IL-2 or IFNγ production from PBMCs, as shown in FIGS. 6G and 6H, suggesting that the activity of CLDN6-1×4-1BB NA and CLDN6-1×4-1BB WT was dependent on the presence of tumor antigen. In contrast, 4-1BB sdAb-Fc was inactive in this assay.

### Example 4. Tumor Growth Inhibition by CLDN6-1×4-1BB BsAbs

CT26 cells, which endogenously express CLDN6 were subcutaneously implanted into BALB/c humanized 4-1BB mice. When tumors grew to an average of 100 mm³, the mice were intraperitoneally treated with (a) human IgG1, (b) CLDN6-1×4-1BB NA (2 mg/kg), (c) CLDN6-1×4-1BB WT (2 mg/kg), or (d) a combination of the parental CLDN6-1 antibody and the 4-1BB sdAb-Fc (1.8 mg/kg and 0.7 mg/kg). The treatments were administered twice weekly, for a total of 6 doses. Tumor growth was monitored by volumetric measurement. As shown in FIGS. 7A and 7B, CLDN6-1×4-1BB WT and CLDN6-1×4-1BB NA both exhibited anti-tumor activity, in which CLDN6-1×4-1BB WT achieved even stronger activity with tumor growth inhibition (TGI) of 75%.

### Example 5. Liver Toxicity Evaluation of CLDN6×4-1BB BsAbs

The major concern of t4-1BB agonist antibody therapy is dose-limiting liver toxicity, as observed in urelumab's clinical development. Most common adverse events were elevated alanine transaminase (ALT), aspartate aminotransferase (AST) and fatigue. Thus, the liver toxicity of CLDN6-1×4-1BB WT and CLDN6-1×4-1BB NA was further evaluated.

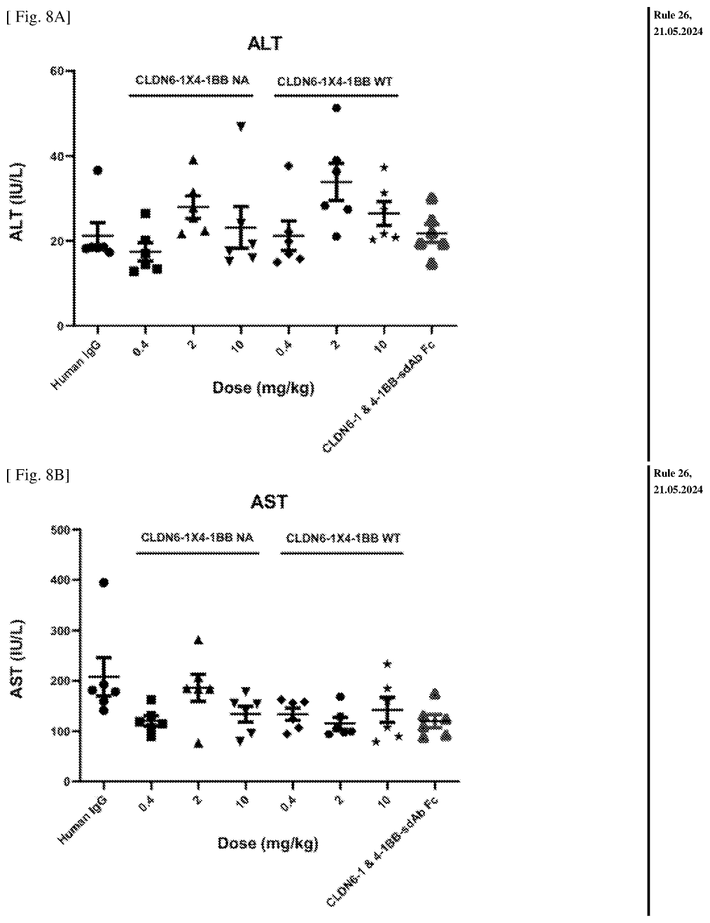

Briefly, blood samples were collected for ALT and AST measurements from hu4-1BB mice after treatment of CLDN6-1×4-1BB WT or CLDN6-1×4-1BB NA at different doses twice weekly. As shown in FIGS. 8A-8B, no significant elevation of ALT and AST was observed, suggesting little risk for liver toxicity commonly induced by other 4-1BB agonist antibodies.

### Example 6. Generation of MUC16 Antibodies

### Phage ELISA and Phage FACS (0VCAR3 Cell) Based Panning

Panning was performed with MUC16 Antigen (human MUC16 AA13789-14451) against human naïve Fab phage library, 13 unique hits C25, C73, D1, D20, D30, D41, D46, D57, D79, D100, B76, B195 and B218 were obtained (Table 4).

TABLE 4
Sequences of the anti-MUC16 mAbs

| | | | SEQ ID |
|---|---|---|---|
| Chain | CDR | Sequence | NO: |
| C25 | VH | EVQLLESGAEVKKPGSSVKVSCKASGGTFSSYAISWVRQAPGQGLEWMGGIIPIFG | 63 |
| | | TANYAQKFQGRVTITADESTSTAYMELSSLRSEDTAVYYCARDSRKYYYDSSGPAL | |
| | | WGFDAFDIWGQGTMVTVSS | |
| | VL | NIQLTQSPSSLSASVGDRVTITCRASQSISSYLNWYQQKPGKAPKLLIYAASSLQS | 64 |
| | | GVPSRFSGSGSGTDFTLTISSLQPEDFATYYCQQSYSTLSTFGPGTKVDIK | |
| | HCDR1 | SYAIS | 57 |
| | HCDR2 | GIIPIFGTANYAQKFQG | 58 |
| | HCDR3 | DSRKYYYDSSGPALWGFDAFDI | 59 |
| | LCDR1 | RASQSISSYLN | 60 |
| | LCDR2 | AASSLQS | 61 |
| | LCDR3 | QQSYSTLST | 62 |
| C73 | VH | QVQLMQSGAEVKKPGSSVKVSCKASGGTFSSYAISWVRQAPGQGLEWMGGIIPIFG | 71 |
| | | TANYAQKFQGRVTITADESTSTAYMELSSLRSEDTAVYYCAREPPLSNYGDYATEQ | |
| | | YYYGMDVWGQGTLVTVSS | |
| | VL | NIQMTQSPSSLSASVGDRVTITCRASQSISSYLNWYQQKPGKAPKLLIYAASSLQS | 72 |
| | | GVPSRFSGSGSGTDFTLTISSLQPEDFATYYCQQSYSTPLTFGGGTKVEIK | |
| | HCDR1 | SYAIS | 57 |
| | HCDR2 | GIIPIFGTANYAQKFQG | 58 |
| | HCDR3 | EPPLSNYGDYATEQYYYGMDV | 67 |
| | LCDR1 | RASQSISSYLN | 60 |
| | LCDR2 | AASSLQS | 61 |
| | LCDR3 | QQSYSTPLT | 70 |
| D1 | VH | QVQLVQSGAEVKKPGSSVKVSCKASGGTFSSYAISWVRQAPGQGLEWMGGIIPIFG | 79 |
| | | TANYAQKFQGRVTITADESTSTAYMELSSLRSEDTAVYYCARAPMVRGVPPTPYYY | |
| | | YYGMDVWGQGTTVTVSS | |
| | VL | EIVLTQSPATLSLSPGERATLSCRASQSVSNYLAWYQQKPGQAPRLLIYDASNRAT | 80 |
| | | GIPARFSGSGSGTDFTLTISSLEPEDFAVYYCQQRSNWPSFGQGTKVEIK | |
| | HCDR1 | SYAIS | 57 |
| | HCDR2 | GIIPIFGTANYAQKFQG | 58 |
| | HCDR3 | APMVRGVPPTPYYYYYGMDV | 75 |
| | LCDR1 | RASQSVSNYLA | 76 |
| | LCDR2 | DASNRAT | 77 |
| | LCDR3 | QQRSNWPS | 78 |
| D20 | VH | QVQLVQSGAEVKKPGSSVKVSCKASGGTFSSYAISWVRQAPGQGLEWMGGIIPIFG | 87 |
| | | TANYAQKFQGRVTITADESTSTAYMELSSLRSEDTAVYYCASTPELLWFGELGGAY | |
| | | YFDYWGQGTLVTVSS | |
| | VL | NIQLTQSPSTLSASVGDRVTITCRASESISSWLAWYQQKPGEAPKLLIYKASTLEN | 88 |
| | | GVPSRFSGSGSETEFTLTISSLQPDDFATYYCQQYRSHWSSTFGPGTKVDIK | |
| | HCDR1 | SYAIS | 57 |
| | HCDR2 | GIIPIFGTANYAQKEQG | 58 |
| | HCDR3 | TPELLWFGELGGAYYFDY | 83 |
| | LCDR1 | RASESISSWLA | 84 |
| | LCDR2 | KASTLEN | 85 |
| | LCDR3 | QQYRSHWSST | 86 |
| D30 | VH | QVQLVQSGAEVKKPGSSVKVSCKASGGTFSSYAISWVRQAPGQGLEWMGGIIPIFG | 95 |
| | | TANYAQKFQGRVTITADESTSTAYMELSSLRSEDTAVYYCARANFNIYYYYYGMDV | |
| | | WGQGTLVTVSS | |
| | VL | DIVMTQSPLSLPVTPGEPASISCRSSQSLLHSNGYNYLDWYLQKPGQSPQLLIYLG | 96 |
| | | SNRASGVPDRFSGSGSGTDFTLKISRVEAEDVGIYYCMQGTHWPRTFGQGTKVEIK | |
| | HCDR1 | SYAIS | 57 |
| | HCDR2 | GIIPIFGTANYAQKEQG | 58 |
| | HCDR3 | ANFNIYYYYYGMDV | 91 |
| | LCDR1 | RSSQSLLHSNGYNYLD | 92 |
| | LCDR2 | LGSNRAS | 93 |
| | LCDR3 | MQGTHWPRT | 94 |
| D41 | VH | EVQLLESGGGLVQPGGSLRLSCAASGFTFSSYEMNWVRQAPGKGLEWVGRIKSKTD | 103 |
| | | GGTTDYAAPVKGRFTISRDDSKNTLYLQMNSLKTEDTAVYYCTTDLAAVAGLEDYW | |
| | | GQGTTVTVSS | |
| | VL | DIQLTQSPSSLSASVGDRVTITCQASQDISNYLNWYQQKPGKAPKLLIYDASNLET | 104 |
| | | GVPSRFSGSGSGTDFTLTISSLQPEDFATYYCQQSYSTPWKFGQGTKVEIK | |
| | HCDR1 | SYEMN | 97 |
| | HCDR2 | RIKSKTDGGTTDYAAPV | 98 |
| | HCDR3 | DLAAVAGLEDY | 99 |
| | LCDR1 | QASQDISNYLN | 100 |
| | LCDR2 | DASNLET | 101 |
| | LCDR3 | QQSYSTPWK | 102 |
| D46 | VH | QVQLVQSGAEVKKPGSSVKVSCKASGGTFSSYAISWVRQAPGQGLEWMGRIIPIFG | 121 |
| | | IANYAQKFQGRVTITADKSTSTAYMELSSLRSEDTAVYYCAITGDYDILTGSYYYG | |
| | | MDVWGQGTTVTVSS | |
| | VL | NIQLTQSPSSLSASVGDRVTITCRASQGIRNDLGWYQQKPGKAPKLLIYAASSLQS | 122 |
| | | GVPSRFSGSGSGTDFTLTISSLQPEDFATYYCLQDYNYPFTFGPGTKVDIK | |
| | HCDR1 | SYAIS | 57 |
| | HCDR2 | RIIPIFGIANYAQKFQG | 106 |
| | HCDR3 | TGDYDILTGSYYYGMDV | 107 |
| | LCDR1 | RASQGIRNDLG | 108 |
| | LCDR2 | AASSLQS | 61 |
| | LCDR3 | LQDYNYPFT | 120 |
| D57 | VH | EVQLLESGGGLVTPGGSLRLSCTASGFTFSDYYLSWVRQAPGKGLEWMGGIIPIFG | 129 |
| | | TANYAQKFQGRVTITADESTSTAYMELSSLRSEDTAVYYCARGGPHYDFWSGYTPG | |
| | | QHGGAFDIWGQGTMVTVSS | |
| | VL | EIVLTQSPGTLSLSPGERATLSCRASQSVSSSYLAWYQQKPGQAPRLLIYGASSRA | 130 |
| | | TGIPDRFSGSGSGTDFTLTISRLEPEDFAVYYCQQRSNWRNTFGQGTRLEIK | |
| | HCDR1 | DYYLS | 123 |
| | HCDR2 | GIIPIFGTANYAQKFQG | 58 |
| | HCDR3 | GGPHYDFWSGYTPGQHGGAFDI | 125 |
| | LCDR1 | RASQSVSSSYLA | 126 |
| | LCDR2 | GASSRAT | 127 |
| | LCDR3 | QQRSNWRNT | 128 |
| D79 | VH | QMQLVQSGAEVKKPGSSVKVSCKASGGTFSSYAISWVRQAPGQGLEWMGGIIPIEG | |
| | | TANYAQKFQGRVTITADESTSTAYMELSSLRSEDTAVYYCARDSGSSITMVRGGDY | 137 |
| | | YYMDVWGKGTTVTVSS | |
| | VL | EIVLTQSPATLSLSPGERATLSCRASQSVSSYLAWYQQKPGQAPRLLIYDASNRAT | 138 |
| | | GIPARFSGSGSGTDFTLTISSLEPEDFAVYYCQQRSNWPPTFGQGTKVEIK | |
| | HCDR1 | SYAIS | 57 |
| | HCDR2 | GIIPIFGTANYAQKFQG | 58 |
| | HCDR3 | DSGSSITMVRGGDYYYMDV | 133 |
| | LCDR1 | RASQSVSSYLA | 134 |
| | LCDR2 | DASNRAT | 77 |
| | LCDR3 | QQRSNWPPT | 136 |
| D100 | VH | QVQLVQSGAEVKKPGSSVKVSCKASGGTFSRYHAISWVRQAPGQGLEWMGGIIPIL | 145 |
| | | GTANYAQKFQGRVTITADESTSTAYMELSSLRSEDTAVYYCASGTTAARYYYYYYY | |
| | | MDVWGKGTTVTVSS | |
| | VL | NIQMTQSPSSLSASVGDRVTITCQASQDISNYLNWFHQKPGKAPNLLIYDASNLET | 146 |
| | | GVPSRFSGSGSGTDFTFTISSLQPEDIGTYYCQQYDNLPLTFGGGTKVDIK | |
| | HCDR1 | YHAIS | 139 |
| | HCDR2 | GIIPILGTANYAQKFQG | 140 |
| | HCDR3 | GTTAARYYYYYYYMDV | 141 |
| | LCDR1 | QASQDISNYLN | 100 |
| | LCDR2 | DASNLET | 101 |
| | LCDR3 | QQYDNLPLT | 144 |
| B76 | VH | QMQLVQSGAEVKKPGSSVKVSCKASGGTFSSYAISWVRQAPGQGLEWMGGIIPIFG | |
| | | TANYAQKFQGRVTITADESTSTAYMELSSLRSEDTAVYYCARSITDYYDSSGYYFR | 153 |
| | | PHFNTGYYYGMDVWGQGTTVTVSS | |
| | VL | NIQLTQSPSSLSASVGDRVTITCRASQGINNYLAWYQQKPGKVPKLLIYAASTLQS | 154 |
| | | GVPSRFSGSGSGTDFTLTITNLQPDDFATYYCQQYDTFSETFGPGTKVEIK | |
| | HCDR1 | SYAIS | 57 |
| | HCDR2 | GIIPIFGTANYAQKFQG | 58 |
| | HCDR3 | SITDYYDSSGYYFRPHFNTGYYYGMDV | 149 |
| | LCDR1 | RASQGINNYLA | 150 |
| | LCDR2 | AASTLQS | 151 |
| | LCDR3 | QQYDTESET | 152 |
| B218 | VH | EVQLLESGAEVKKPGSSVKVSCKASGGTFSSYAISWVRQAPGQGLEWMGGIIPIFG | |
| | | TANYAQKFQGRVTITADESTSTAYMELSSLRSEDTAVYYCARGGPHYDFWSGYTPG | 161 |
| | | QHGGAFDIWGQGTMVTVSS | |
| | VL | NIQMTQSPSTLSASLGDRVSITCRASQSISGWLAWYQQKPGKAPKLLIYRTSYLES | 162 |
| | | GVPSRESAGGSGTEFTLTISSLQPDDFATYYCQHYDTFSRAFGQGTKVDIK | |
| | HCDR1 | SYAIS | 57 |
| | HCDR2 | GIIPIFGTANYAQKFQG | 58 |
| | HCDR3 | GGPHYDFWSGYTPGQHGGAFDI | 125 |
| | LCDR1 | RASQSISGWLA | 158 |
| | LCDR2 | RTSYLES | 159 |
| | LCDR3 | QHYDTFSRA | 160 |
| B195 | VH | EVQLVQSGAEVKKPGASVKVSCKASGGTFSSYAISWVRQAPGQGLEWMGGIIPIFG | 169 |
| | | TANYAQKFQGRVTITADESTSTAYMELSSLRSEDTAVYYCAREGPDYGDYSWSMDY | |
| | | YYGMDVWGQGTMVTVSS | |
| | VL | EIVMTQSPGTLSLSPGERATLSCRASQSVNSRYLAWYQHKPGQAPRLLMYGASTRA | 170 |
| | | TGIPDRFSGSGSGTDFTLTISRLEPEDFAVYYCQQYGTFSITFGQGTRLEIK | |
| | HCDR1 | SYAIS | 57 |
| | HCDR2 | GIIPIFGTANYAQKFQG | 58 |
| | HCDR3 | EGPDYGDYSWSMDYYYGMDV | 165 |
| | LCDR1 | RASQSVNSRYLA | 166 |
| | LCDR2 | GASTRAT | 167 |
| | LCDR3 | QQYGTESIT | 168 |

### Example 7. Binding Activity of MUC16 mAbs

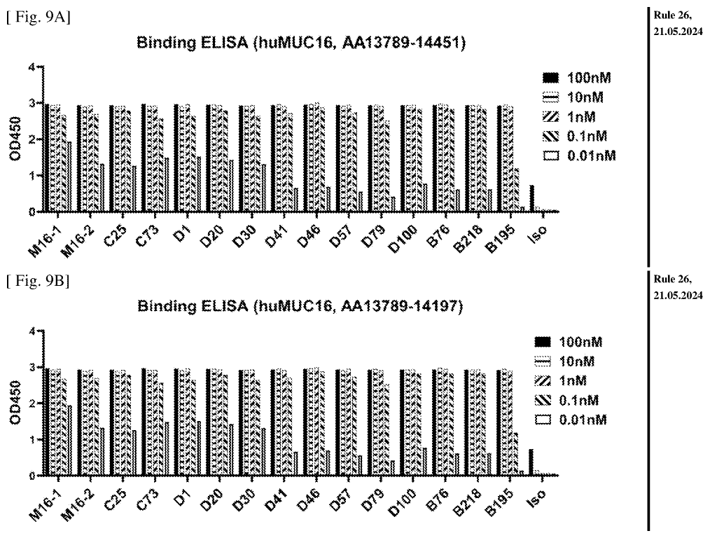

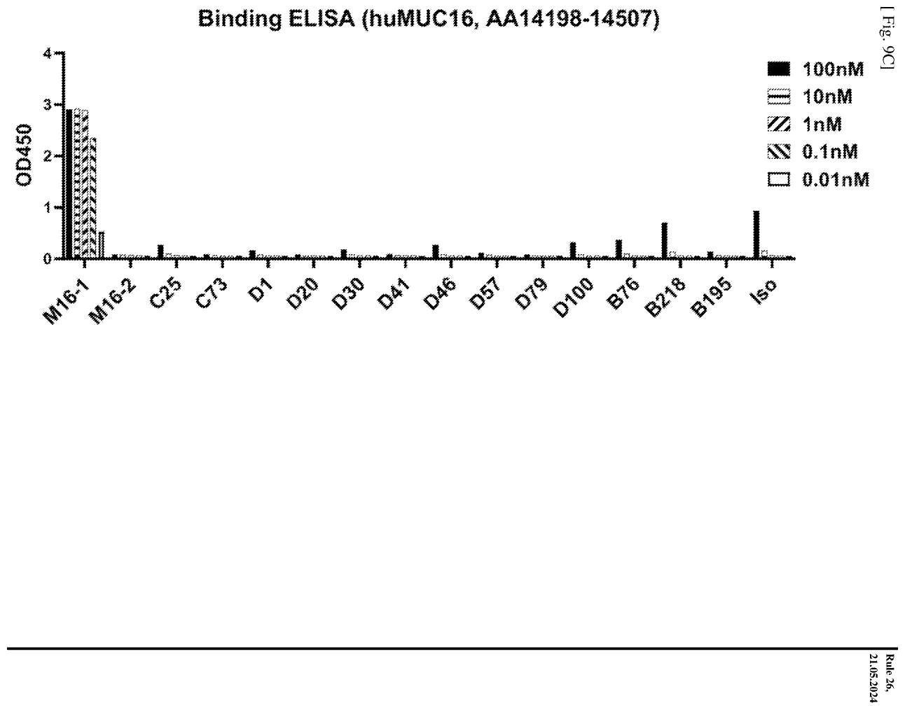

The binding activity of the MUC16 mAbs were tested via ELISA using different fragments of human MUC16 antigen (FIGS. 9A-9C). Similar to M16-2, but different from M16-1, all the 13 mAbs binds to AA13789-14197 domain of human MUC16. M16-1 and M16-2 are benchmarker antibodies with sequences listed in Table of Amino Acid Sequences A.

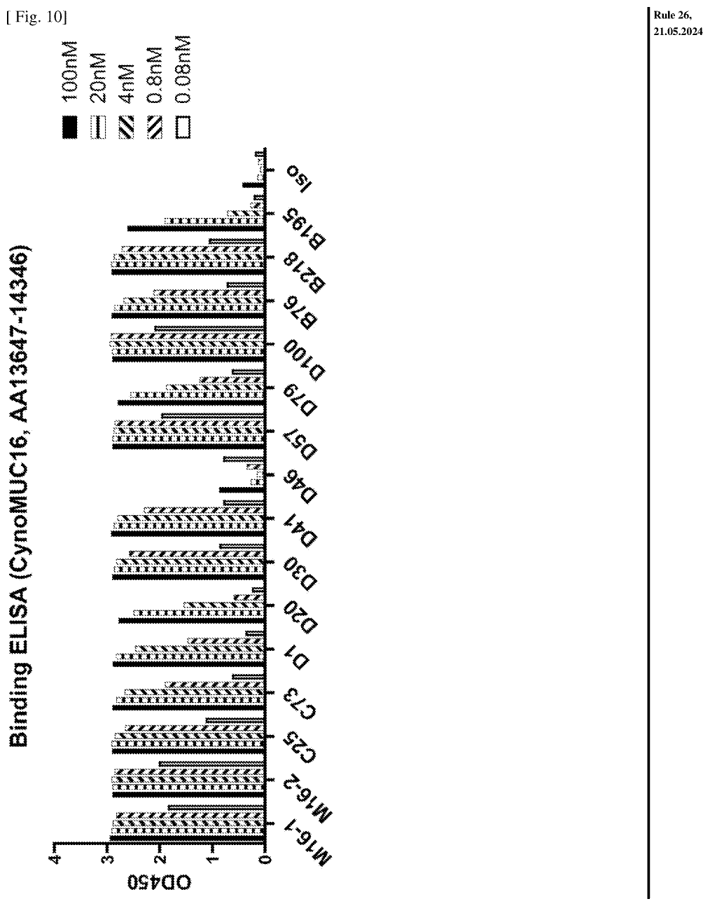

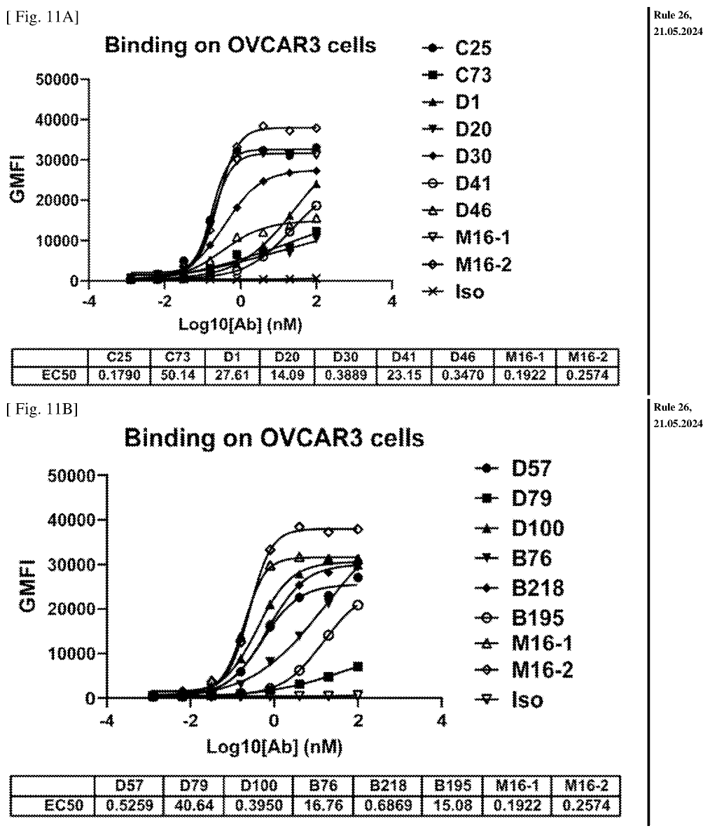

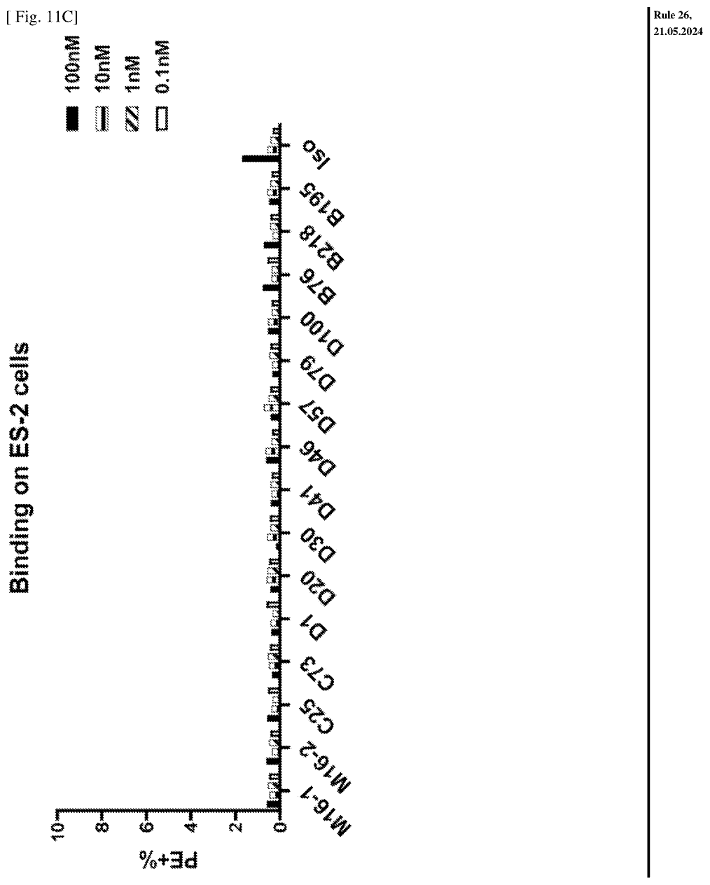

Cross species tests showed that except for D46, the other 12 mAbs are able to cross react with cyno MUC16 (FIG. 10).

OVCAR3 has the highest level of endogenous MUC16 expression. In the cellular binding assay, all the 13 mAbs (human IgG1 Fc) bind to OVCAR3 cells (FIGS. 11A-11B). In contrast, the 13 mAbs (human IgG1 Fe) do not bind to ES-2 cell, which is MUC16 negative (FIG. 11C).

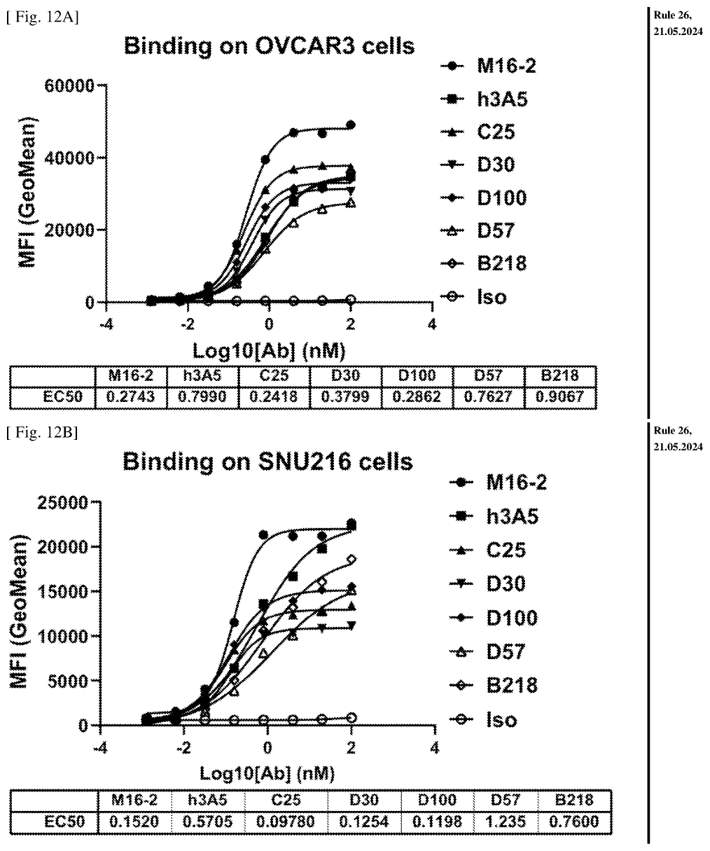

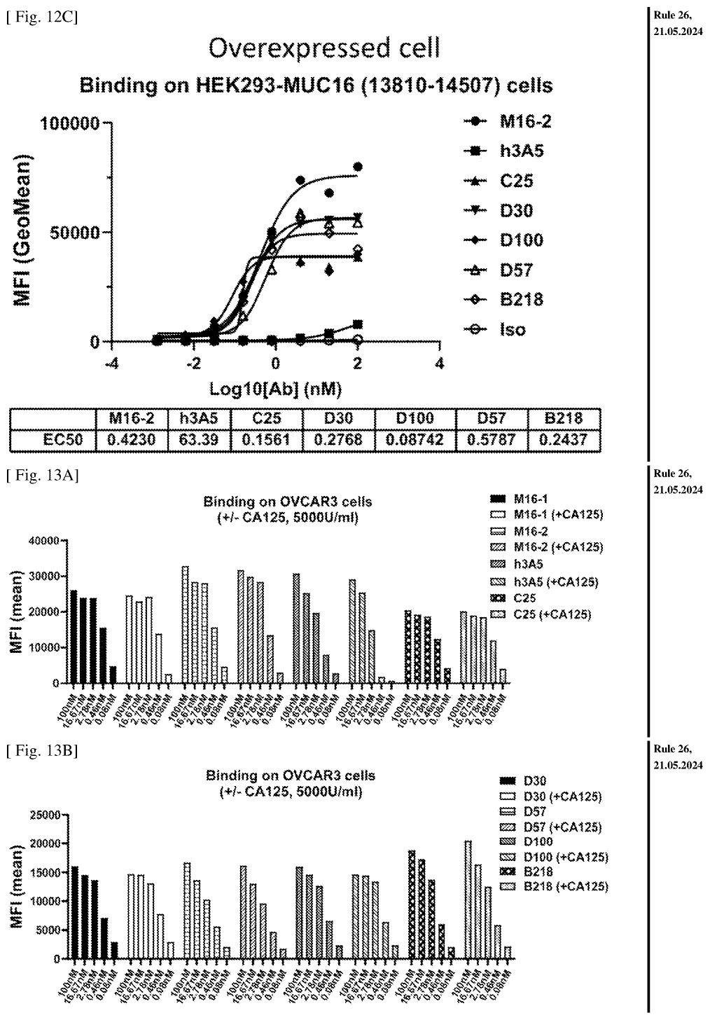

For the five selected clones C25, D30, D100, D57 and B218, cell-based binding assay was performed (FIGS. 12A-12C). All the mAbs showed effective binding to the huMUC16 in the cells, whereas h3A5 does not bind HEK293-MUC16 (13810-14507) (FIGS. 12A-12C). Among the three human MUC16 positive cell lines, OVCAR3 has the highest level of endogenous MUC16 expression. M16-2 and 3A5 are benchmarker antibodies with sequences listed in Table of Amino Acid Sequences A.

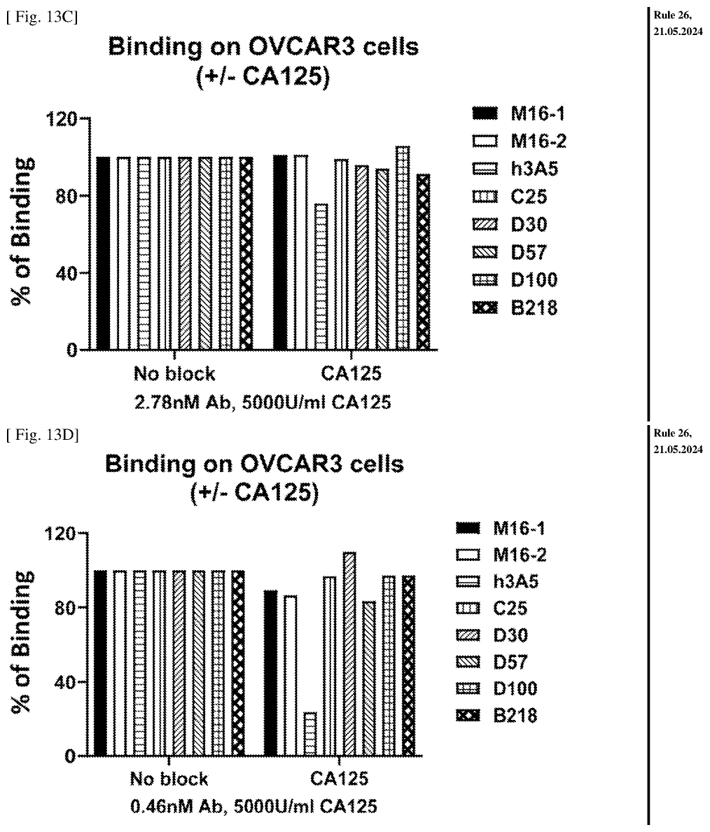

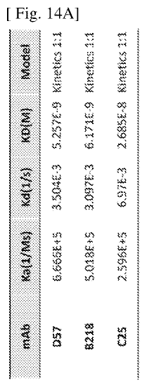

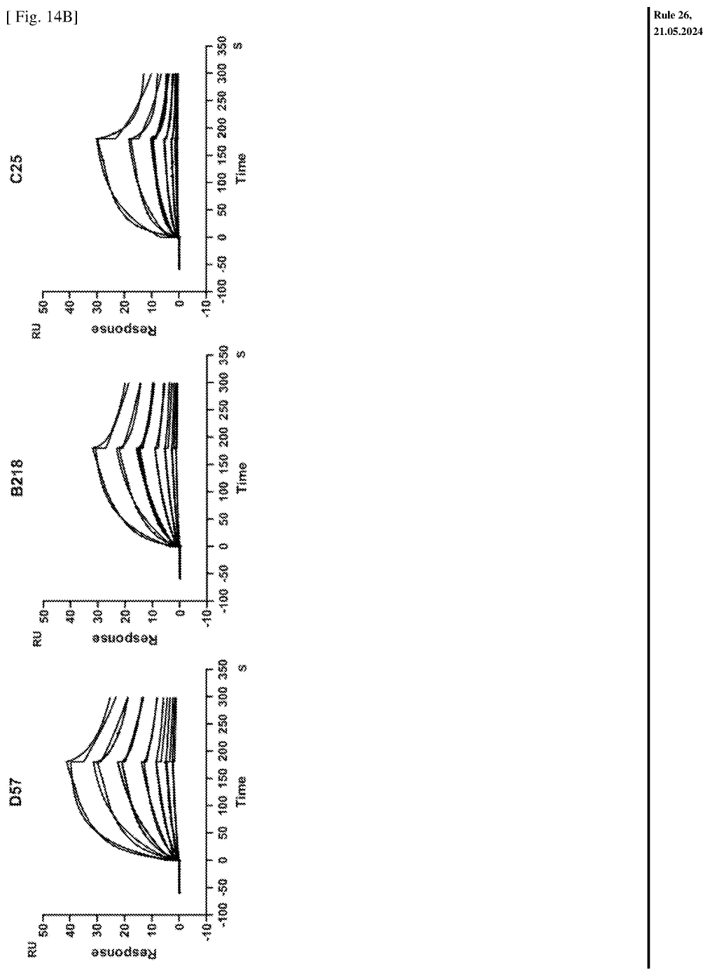

In another cell-based binding assay, where patient ascites derived soluble CA125 (Carcinoma antigen 125, an extracellular shed protein encoded by the MUC 16 gene, a serum marker used routinely to monitor patients with ovarian cancer) was present or absent. The binding ability of the 5 mAbs to OVCAR3 are not markedly decreased by addition of soluble CA125, indicating that the mAbs binding to a different epitope than CA125. However, the binding of benchmarker 3A5 to OVCAR3 is greatly decreased by addition of 5000 U/mL soluble CA125 (FIGS. 13A-13D).

The kinetics of the binding assay showed K_{D} values of D57, B218 and C25, respectively (FIGS. 14A-14B).

### Example 8. MUC16×4-1BB Bispecific Antibodies

The exemplary MUC16×4-1BB bispecific antibodies shown in Table 5 below were designed and generated.

TABLE 5
Exemplary MUC16 x 4-1BB Bispecific Antibodies
D57-4B: MUC16-D57 IgG1 N297A X 4-1BB
(MUC16 X 4-1BB NA, 4-1BB VHH fused to C-terminus of heavy chain of MUC16-D57 antibody
moiety, with N297A in IgG1 Fc region)

| | | |
|---|---|---|
| Heavy chain | (SEQ ID NO: 129) | EVQLLESGGGLVTPGGSLRLSCTASGFTFSDYYLSWVRQAPGKGLEWMGGIIPIF |
| (SEQ ID NO: | MUC16-D57 VH | GTANYAQKFQGRVTITADESTSTAYMELSSLRSEDTAVYYCARGGPHYDFWSGYT |
| 174) | | PGQHGGAFDIWGQGTMVTVSS |
| | IgG heavy chain | ASTKGPSVFPLAPSSKSTSGGTAALGCLVKDYFPEPVTVSWNSGALTSGVHTFPA |
| | constant region | VLQSSGLYSLSSVVTVPSSSLGTQTYICNVNHKPSNTKVDKKVEPKSCDKTHTCP |
| | (IgG(CH)) (SEQ ID | PCPAPELLGGPSVFLFPPKPKDTLMISRTPEVTCVVVDVSHEDPEVKENWYVDGV |
| | NO: 172) | EVHNAKTKPREEQYASTYRVVSVLTVLHQDWLNGKEYKCKVSNKALPAPIEKTIS |
| | | KAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| | Linker (SEQ ID | GGGGSGGGGSGGGGSGGGGSGGGGS |
| | NO: 171) | |
| | 4-1BB VHH (SEQ | QVQLQESGGGLVQPGGSLRLSCAASGYTYSSNCMGWFRQAPGKGLEGVAVICTGG |
| | ID NO: 27) | GSPSYADSVKGRFTISRDNAKNTLYLQMNSLRAEDTAVYYCAADLLRAGTPLSSY |
| | | EFNYWGQGTLVTVSS |
| Light chain | MUC16-D57 VL | EIVLTQSPGTLSLSPGERATLSCRASQSVSSSYLAWYQQKPGQAPRLLIYGASSR |
| component | (SEQ ID NO: 130) | ATGIPDRFSGSGSGTDFTLTISRLEPEDFAVYYCQQRSNWRNTFGQGTRLEIK |
| (SEQ ID NO: | IgG light chain | RTVAAPSVFIFPPSDEQLKSGTASVVCLLNNFYPREAKVQWKVDNALQSGNSQES |
| 175) | constant region | VTEQDSKDSTYSLSSTLTLSKADYEKHKVYACEVTHQGLSSPVTKSENRGEC |
| | (IgG(CL)) (SEQ ID | |
| | NO: 173) | |

B218-4B: MUC16-B218 IgG1 N297A X 4-1BB
(MUC16-B218 X 4-1BB NA, 4-1BB VHH fused to C-terminus of heavy chain of MUC16-B218 antibody
moiety, with N297A in IgG1 Fc region)

| | | |
|---|---|---|
| Heavy chain | MUC16-D218 VH | EVQLLESGAEVKKPGSSVKVSCKASGGTFSSYAISWVRQAPGQGLEWMGGIIPIF |
| component | (SEQ ID NO: 161) | GTANYAQKFQGRVTITADESTSTAYMELSSLRSEDTAVYYCARGGPHYDFWSGYT |
| (SEQ ID NO: | | PGQHGGAFDIWGQGTMVTVSS |
| 176) | IgG(CH) (SEQ ID | ASTKGPSVFPLAPSSKSTSGGTAALGCLVKDYFPEPVTVSWNSGALTSGVHTFPA |
| | NO: 172) | VLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFNWYVDGV |
| | | EVHNAKTKPREEQYASTYRVVSVLTVLHQDWLNGKEYKCKVSNKALPAPIEKTIS |
| | | KAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| | Linker (SEQ ID | GGGGSGGGGSGGGGSGGGGSGGGGS |
| | NO: 171) | |
| | 4-1BB VHH (SEQ | QVQLQESGGGLVQPGGSLRLSCAASGYTYSSNCMGWFRQAPGKGLEGVAVICTGG |
| | ID NO: 27) | GSPSYADSVKGRFTISRDNAKNTLYLQMNSLRAEDTAVYYCAADLLRAGTPLSSY |
| | | EFNYWGQGTLVTVSS |
| Light chain | MUC16-D218 VL | NIQMTQSPSTLSASLGDRVSITCRASQSISGWLAWYQQKPGKAPKLLIYRTSYLE |
| component | (SEQ ID NO: 162) | SGVPSRESAGGSGTEFTLTISSLQPDDFATYYCQHYDTFSRAFGQGTKVDIK |
| (SEQ ID NO: | IgG(CL) (SEQ ID | RTVAAPSVFIFPPSDEQLKSGTASVVCLLNNFYPREAKVQWKVDNALQSGNSQES |
| 177) | NO: 173) | VTEQDSKDSTYSLSSTLTLSKADYEKHKVYACEVTHQGLSSPVTKSENRGEC |

C25-4B: MUC16-C25 IgG1 N297A X 4-1BB
(CLDN6-1 X 4-1BB NA, 4-1BB VHH fused to C-terminus of heavy chain of MUC16-C25 antibody
moiety, with N297A in IgG1 Fc region)

| | | |
|---|---|---|
| Heavy chain | MUC16-C25 VH | EVQLLESGAEVKKPGSSVKVSCKASGGTFSSYAISWVRQAPGQGLEWMGGIIPIF |
| component | (SEQ ID NO: 63) | GTANYAQKFQGRVTITADESTSTAYMELSSLRSEDTAVYYCARDSRKYYYDSSGP |
| (SEQ ID NO: | | ALWGFDAFDIWGQGTMVTVSS |
| 178) | IgG(CH) (SEQ ID | ASTKGPSVFPLAPSSKSTSGGTAALGCLVKDYFPEPVTVSWNSGALTSGVHTFPA |
| | NO: 172) | VLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFNWYVDGV |
| | | EVHNAKTKPREEQYASTYRVVSVLTVLHQDWLNGKEYKCKVSNKALPAPIEKTIS |
| | | KAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| | Linker (SEQ ID | GGGGSGGGGSGGGGSGGGGSGGGGS |
| | NO: 171) | |
| | 4-1BB VHH (SEQ | QVQLQESGGGLVQPGGSLRLSCAASGYTYSSNCMGWFRQAPGKGLEGVAVICTGG |
| | ID NO: 27) | GSPSYADSVKGRFTISRDNAKNTLYLQMNSLRAEDTAVYYCAADLLRAGTPLSSY |
| | | EFNYWGQGTLVTVSS |
| Light chain | MUC16-C25 VL | NIQLTQSPSSLSASVGDRVTITCRASQSISSYLNWYQQKPGKAPKLLIYAASSLQ |
| component | (SEQ ID NO: 64) | SGVPSRFSGSGSGTDFTLTISSLQPEDFATYYCQQSYSTLSTFGPGTKVDIK |
| (SEQ ID NO: | IgG(CL) (SEQ ID | RTVAAPSVFIFPPSDEQLKSGTASVVCLLNNFYPREAKVQWKVDNALQSGNSQES |
| 179) | NO: 173) | VTEQDSKDSTYSLSSTLTLSKADYEKHKVYACEVTHQGLSSPVTKSENRGEC |

D100-4B: MUC16-D100 IgG1 N297A X 4-1BB
(CLDN6-1 X 4-1BB NA, 4-1BB VHH fused to C-terminus of heavy chain of MUC16-D100 antibody
moiety, with N297A in IgG1 Fc region)

| | | |
|---|---|---|
| Heavy chain | MUC16- D100 VH | QVQLVQSGAEVKKPGSSVKVSCKASGGTFSRYHAISWVRQAPGQGLEWMGGIIPI |
| component | (SEQ ID NO: 145) | LGTANYAQKFQGRVTITADESTSTAYMELSSLRSEDTAVYYCASGTTAARYYYYY |
| (SEQ ID NO: | | YYMDVWGKGTTVTVSS |
| 180) | IgG(CH) (SEQ ID | ASTKGPSVFPLAPSSKSTSGGTAALGCLVKDYFPEPVTVSWNSGALTSGVHTFPA |
| | NO: 172) | VLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKENWYVDGV |
| | | EVHNAKTKPREEQYASTYRVVSVLTVLHQDWLNGKEYKCKVSNKALPAPIEKTIS |
| | | KAKGQPREPQVYTLPPSREEMTKNQVSLTCLVKGFYPSDIAVEWESNGQPENNYK |
| | | TTPPVLDSDGSFFLYSKLTVDKSRWQQGNVFSCSVMHEALHNHYTQKSLSLSPGK |
| | Linker (SEQ ID | GGGGSGGGGSGGGGSGGGGSGGGGS |
| | NO: 171) | |
| | 4-1BB VHH (SEQ | QVQLQESGGGLVQPGGSLRLSCAASGYTYSSNCMGWFRQAPGKGLEGVAVICTGG |
| | ID NO: 27) | GSPSYADSVKGRFTISRDNAKNTLYLQMNSLRAEDTAVYYCAADLLRAGTPLSSY |
| | | EFNYWGQGTLVTVSS |
| Light chain | MUC16-D100 VL | NIQMTQSPSSLSASVGDRVTITCQASQDISNYLNWFHQKPGKAPNLLIYDASNLE |
| component | (SEQ ID NO: 146) | TGVPSRFSGSGSGTDFTFTISSLQPEDIGTYYCQQYDNLPLTFGGGTKVDIK |
| (SEQ ID NO: | IgG(CL) (SEQ ID | RTVAAPSVFIFPPSDEQLKSGTASVVCLLNNFYPREAKVQWKVDNALQSGNSQES |
| 181) | NO: 173) | VTEQDSKDSTYSLSSTLTLSKADYEKHKVYACEVTHQGLSSPVTKSENRGEC |

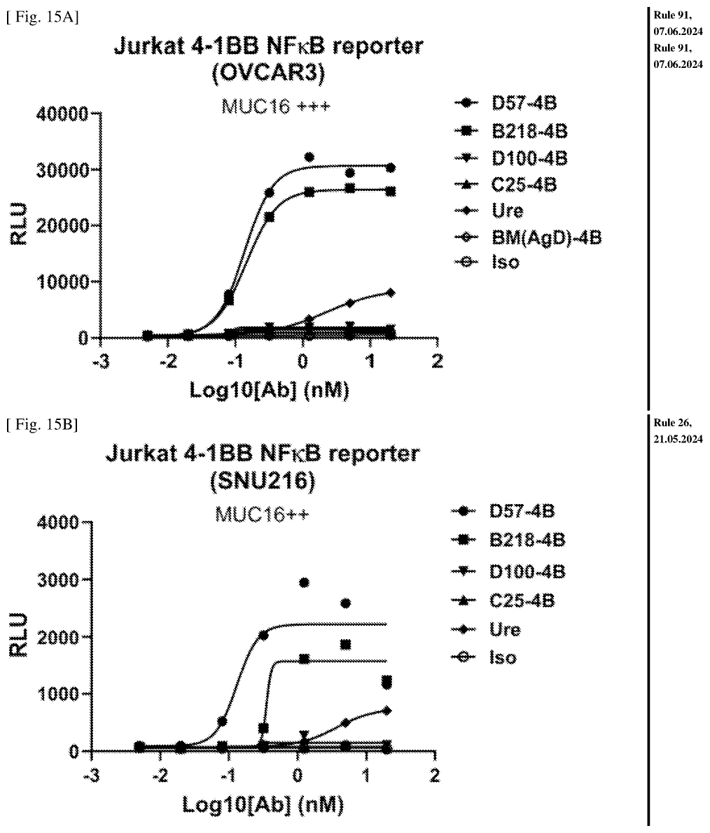

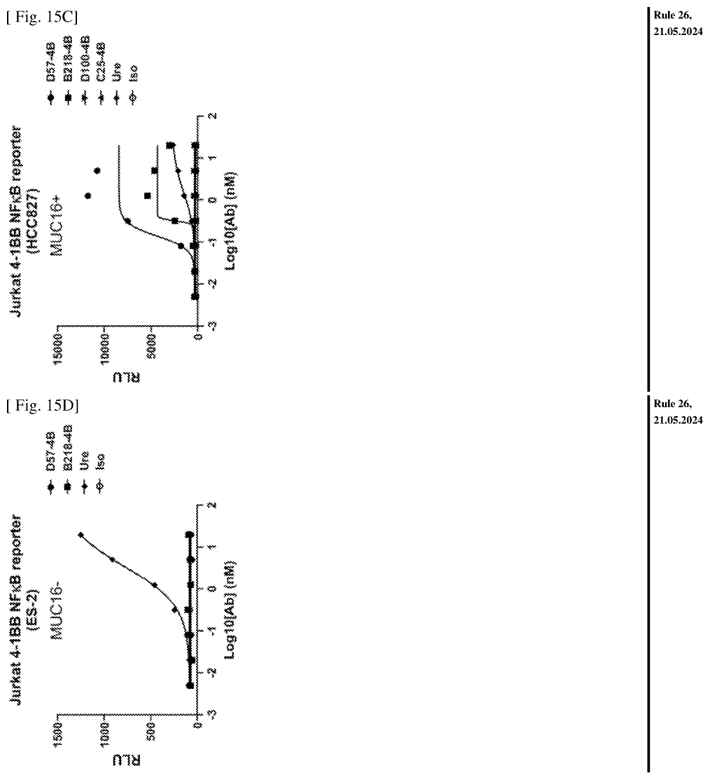

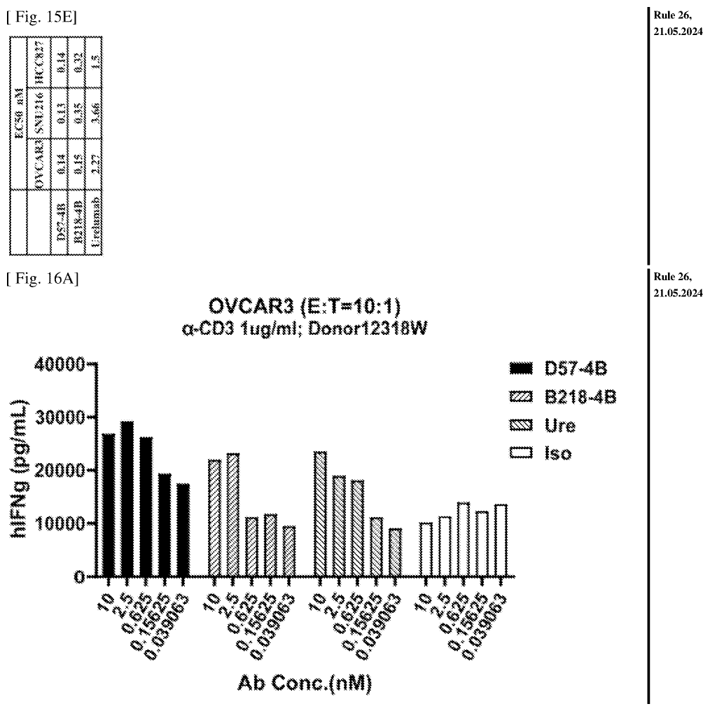

In a 4-11DB NFκB reporter assay, the D5′7-4B3, B3218-4B3 BsAbs could stimulate 4-1DBB signal in the presence of MUC16 high (+++, OVCAR3), medium (++, SNU216) and low (±, HCC827) expression cells, with much stronger signal than Urelumab (FIGS. 15A-15C). In contrast, in the negative cell (ES-2), Urelumab can stimulate 4-11DB activation, while BsAbs hardly induced activation signal (FIG. 15D).

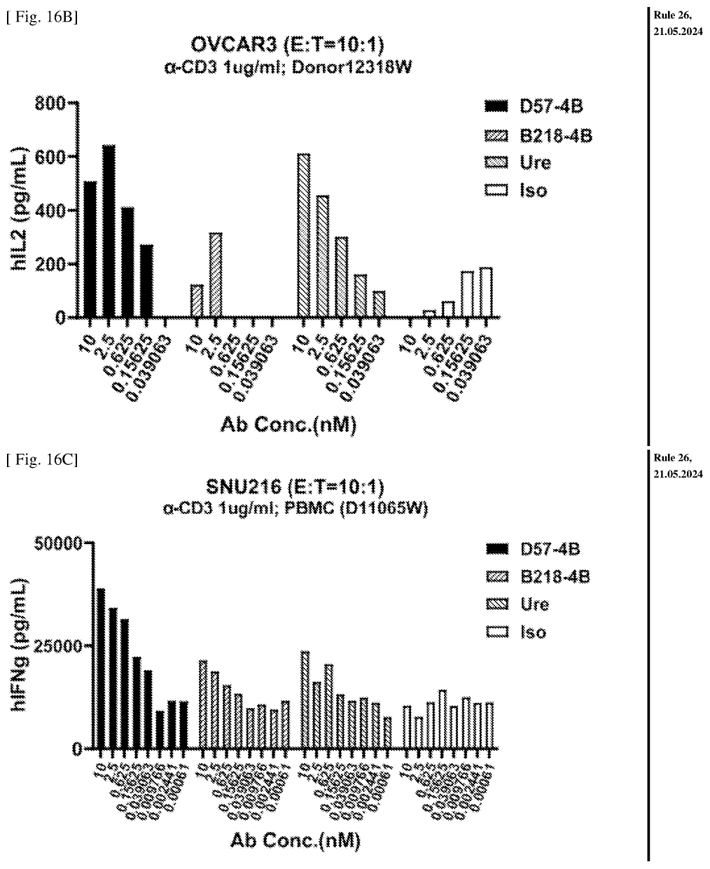

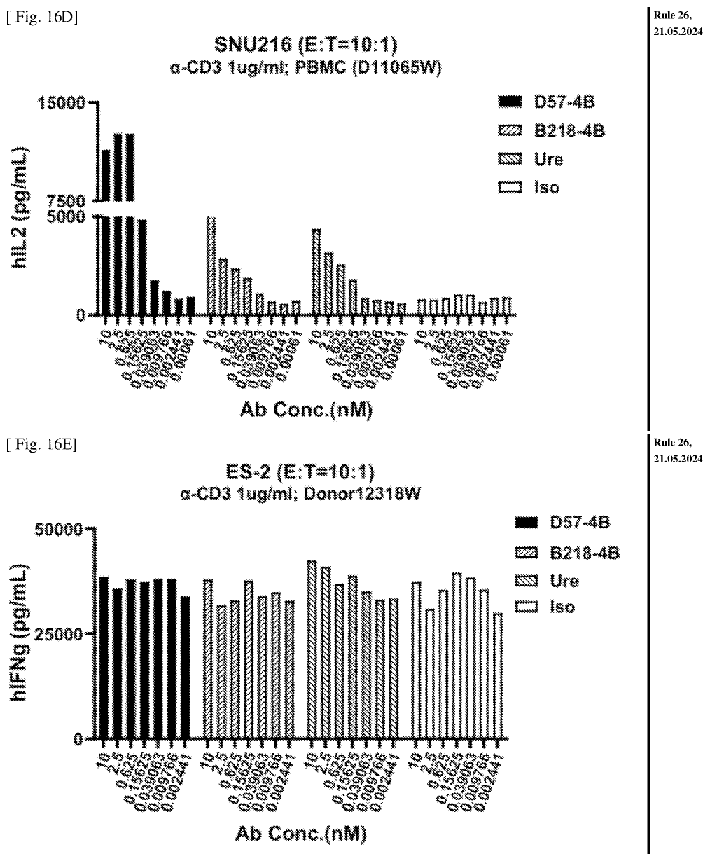

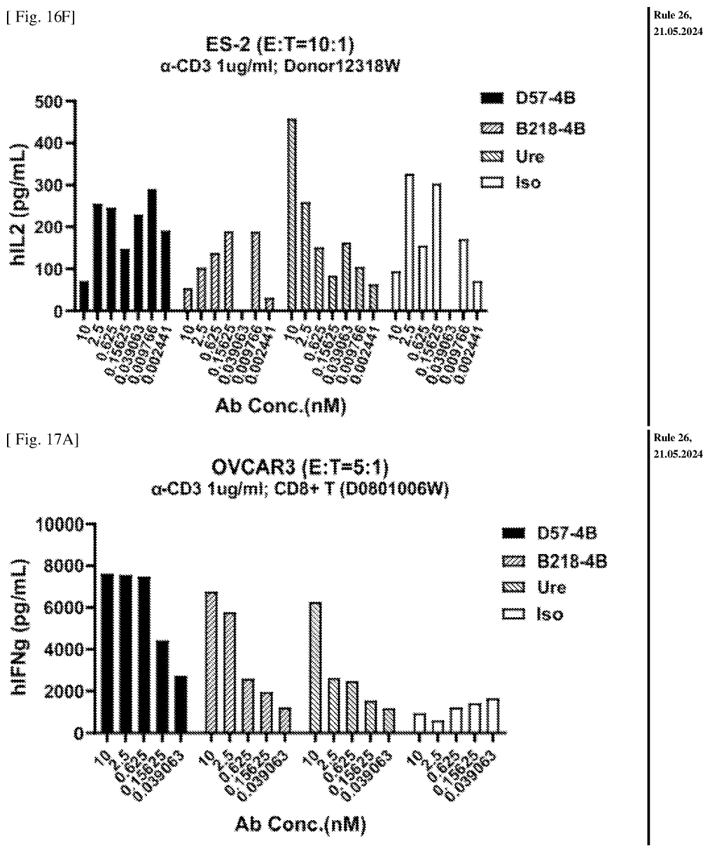

In another 4-1BB BsAb activity assay for measuring PBMC cytokine release in the MUC 16 high (+++, OVCAR3), medium (++, SNU216) and low (+, HCC827) expression cells, D57-4B3 showed strongest T cell costimulatory activity (induce human IFNγ and IL-2 cytokine release), and B3218-4B3 showed comparable T cell costimulatory activity to Urelumab (FIGS. 16A-16F).

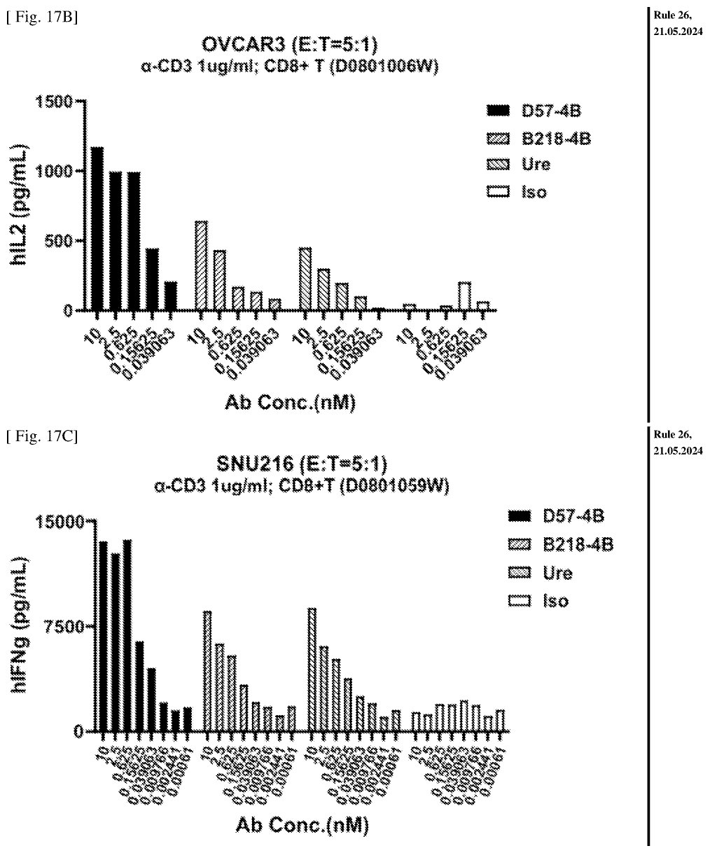

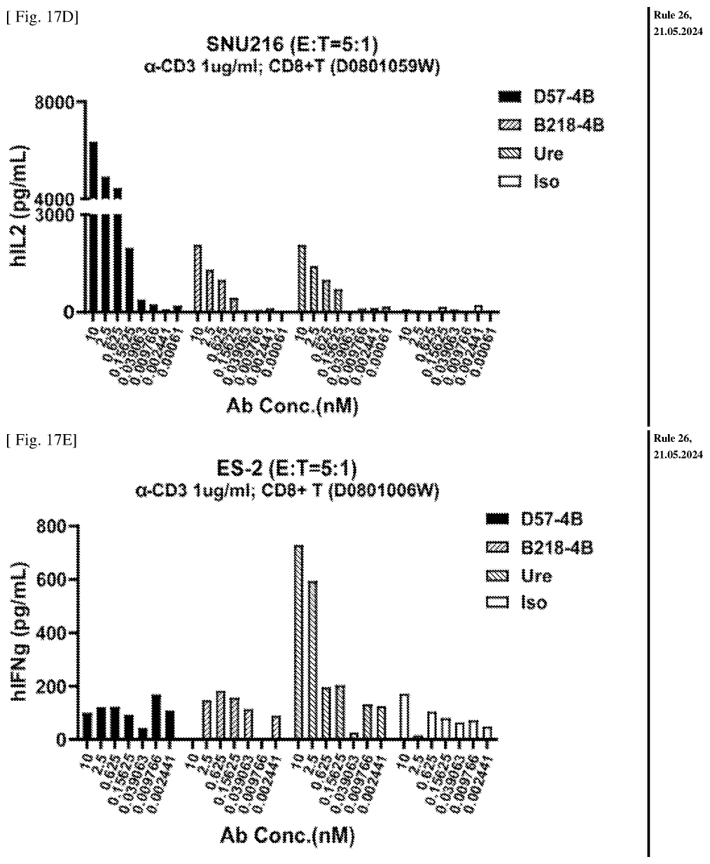

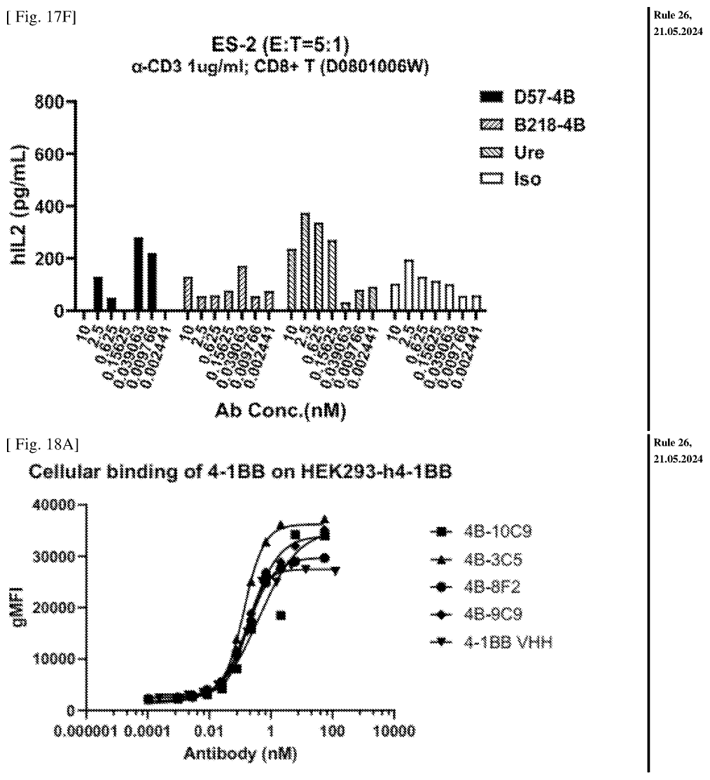

In a further 4-11B BsAb activity assay for measuring CD8+ T cell cytokine release in the MUC16 high (+++, OVCAR3), medium (++, SNU216) and low (+, HCC827) expression cells, D5′7-4B3 showed strongest CD8+ T cell costimulatory activity (induce human IFN7 and IL-2 cytokine release), and BT218-4B showed comparable CD+ T cell costimulatory activity to Urelumab (FIGS. 17A-17F).

### Example 9. Generation of Bispecific and Bi-Paratopic ROR1×4-1BB Antibodies

ROR1×4-1BB bispecific and bi-paratopic antibodies are constructed as shown in Table 6A below.

TABLE 6A
Designed bispecific and tri-specific antibody sequences

| | | |
|---|---|---|
| Chain | Domain | Amino Acid sequence |

4B-3C5
3C5 IgG x 4-1BB VHH

| | | |
|---|---|---|
| HC | 3C5 VH | EVKLVESGGGLVKPGGSLKLSCAASGFTFSSYAMSWVRQTPEKRLEWVSSISSGGTTYYPDTV |
| (SEQ ID | | KGRFTISRDNARNILYLQMSSLRSEDTAMYYCARDSLYYGSSLYYAMDYWGQGTSVTVSS |
| NO: 261) | | (SEQ ID NO: 188) |
| | IgG heavy | ASTKGPSVFPLAPSSKSTSGGTAALGCLVKDYFPEPVTVSWNSGALTSGVHTFPAVLQSSGLY |
| | chain constant | SLSSVVTVPSSSLGTQTYICNVNHKPSNTKVDKKVEPKSCDKTHTCPPCPAPELLGGPSVFLF |
| | region (IgG | PPKPKDTLMISRTPEVTCVVVDVSHEDPEVKFNWYVDGVEVHNAKTKPREEQYNSTYRVVSVL |
| | (CH)) | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5 VL | DIKMTQSPSSMYASLGERVSITCRASQDIYSYLSWFQQKPGKSPKTLIYRANRLVDGVPSRFS |
| (SEQ ID | | GSGSGQDYSLTISSLEYEDMGIYYCLQYDEFPYTFGGGTKLEIK (SEQ ID NO: 189) |
| NO: 262) | IgG light | STYSLSSTLTLSKADYEKHKVYACEVTHQGLSSPVTKSENRGEC (SEQ ID NO: 258) |
| | chain constant | RTVAAPSVFIFPPSDEQLKSGTASVVCLLNNFYPREAKVQWKVDNALQSGNSQESVTEQDSKD |
| | region (IgG | |
| | (CL)) | |

4B-8F2
8F2 IgG x 4-1BB VHH

| | | |
|---|---|---|
| HC | 8F2 VH | EVQLQQSGPELVKPGASVKMSCKASGYTFTTYVMHWVKQKPGQGLEWIGYINPYNGGIRYNEK |
| (SEQ ID | | FKGKATLTSDKSSSTAYMELSSLTSEDSAVYYCARRERGVYYGMDEWGQGTSVTVSS (SEQ |
| NO: 263) | | ID NO: 196) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2 VL | DVVMTQTPLTLSVTIGQPASISCKSSQSLLHSNGKTYLNWLLQRPGQSPNLLIYLVSKLESGV |
| (SEQ ID | | PDRFSGSGSGTDFTLKISRVEAEDLGVYYCLQATYFPYTFGSGTKLEIK (SEQ ID NO: |
| NO: 264) | | 197) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC (SEQ ID NO: 258) |

4B-10C9
10C9 IgG x 4-1BB VHH

| | | |
|---|---|---|
| HC | 10C9 VH | QVQLQQPGAELVMPGASVKMSCKASGYKFTDYWMHWVKQRPGQGLEWIGAIDTSDSSTRNNQK |
| (SEQ ID | | FKGKATLTVDESSSTAYMQLSRLTSEDSAVYYCARGARTGTGEGYWGQGTLVTVSS (SEQ |
| NO: 265) | | ID NO: 212) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKENWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 10C9 VL | DVVMTQTPLTLSVAIGQPASISCKSSQSLLHINGKTYLNWLLQRPGQSPKLLIYLVSKLESGV |
| (SEQ ID | | PDRFSGSGSGTDFTLKISRVEAEDLGVYYCLQATHEPYTFGSGTKLEIK (SEQ ID NO: |
| NO: 266) | | 213) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-10C9
10C9 IgG x 4-1BB VHH

| | | |
|---|---|---|
| HC | 9C9 VH | EVKLVESGPVLVAPSQSLSITCTVSGFSLTSYGVHWVRQPPGKGLEWLGVIWAGGHTNYNSDL |
| (SEQ ID | | MSRLSISKDNSKSQVFLKMNSLQTDDTAMYYCVKRSIYGDYALDYWGQGTSVTVSS (SEQ |
| NO: 267) | | ID NO: 204) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKENWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 9C9 VL | DIVLTQSPAITAASLGQKVTITCSASSSVTYMHWYQQKSGTSPKLWIYDISKLASGVPARFSG |
| (SEQ ID | | SGPGTSYSLTISTMEAEDAAIYYCQQWNYPLMTFGGGTKLEIK (SEQ ID NO: 205) |
| NO: 268) | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC (SEQ ID NO: 258) |

4B-BIR1
CH1/CL (3C5 IgG x 4-1BB VHH) +CH2 (8F2 scFv-IgG′(CH)-4-1BB VHH)

| | | |
|---|---|---|
| HC1 | 3C5 VH | EVKLVESGGGLVKPGGSLKLSCAASGFTFSSYAMSWVRQTPEKRLEWVSSISSGGTTYYPDTV |
| (SEQ ID | | KGRFTISRDNARNILYLQMSSLRSEDTAMYYCARDSLYYGSSLYYAMDYWGQGTSVTVSS |
| NO: 269) | | (SEQ ID NO: 188) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHT |
| | | CPPCPAPELLGGPSVFLFPPKPKDTLMISRTPEVTCVVVDVSHEDPEVKENWYVDGVEVHNAK |
| | | TKPREEQYNSTYRVVSVLTVLHQDWLNGKEYKCKVSNKALPAPIEKTISKAKGQPREPQVYTL |
| | | PPCREEMTKNQVSLWCLVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 253) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| HC2 | 8F2 scFv (VL- | DVVMTQTPLTLSVTIGQPASISCKSSQSLLHSNGKTYLNWLLQRPGQSPNLLIYLVSKLESGV |
| (SEQ ID | VH) | PDRFSGSGSGTDFTLKISRVEAEDLGVYYCLQATYFPYTFGSGTKLEIK (SEQ ID NO: |
| NO: 270) | | 197) |
| | | GGGGSGGGGSGGGGS (SEQ ID NO: 23) |
| | | EVQLQQSGPELVKPGASVKMSCKASGYTFTTYVMHWVKQKPGQGLEWIGYINPYNGGIRYNEK |
| | | FKGKATLTSDKSSSTAYMELSSLTSEDSAVYYCARRERGVYYGMDEWGQGTSVTVSS (SEQ |
| | | ID NO: 196) |
| | IgG′ (CH) | AAEPKSSDKTHT |
| | | CPPCPAPELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPREPQVCTL |
| | | PPSREEMTKNQVSLSCAVKGFYPSDIAVEWESNGQPENNYKTTPPVLDSDGSFFLVSKLTVDK |
| | | SRWQQGNVFSCSVMHEALHNHYTQKSLSLSPGK (SEQ ID NO: 255) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5 VL | DIKMTQSPSSMYASLGERVSITCRASQDIYSYLSWFQQKPGKSPKTLIYRANRLVDGVPSRFS |
| (SEQ ID | | GSGSGQDYSLTISSLEYEDMGIYYCLQYDEFPYTFGGGTKLEIK (SEQ ID NO: 189) |
| NO: 271) | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-BIR2
8F5 scFv-3C5 IgG x 4-1BB VHH

| | | |
|---|---|---|
| HC | 8F2 scFv (VL- | DVVMTQTPLTLSVTIGQPASISCKSSQSLLHSNGKTYLNWLLQRPGQSPNLLIYLVSKLESGV |
| (SEQ ID | VH) | PDRFSGSGSGTDFTLKISRVEAEDLGVYYCLQATYFPYTFGSGTKLEIK (SEQ ID NO: |
| NO: 272) | | 197) |
| | | GGGGGGGGSGGGGS (SEQ ID NO: 23) |
| | | EVQLQQSGPELVKPGASVKMSCKASGYTFTTYVMHWVKQKPGQGLEWIGYINPYNGGIRYNEK |
| | | FKGKATLTSDKSSSTAYMELSSLTSEDSAVYYCARRERGVYYGMDEWGQGTSVTVSS (SEQ |
| | | ID NO: 196) |
| | Linker | GGGGSGGGGSGGGGS (SEQ ID NO: 23) |
| | 3C5 VH | EVKLVESGGGLVKPGGSLKLSCAASGFTFSSYAMSWVRQTPEKRLEWVSSISSGGTTYYPDTV |
| | | KGRFTISRDNARNILYLQMSSLRSEDTAMYYCARDSLYYGSSLYYAMDYWGQGTSVTVSS |
| | | (SEQ ID NO: 188) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5 VL | DIKMTQSPSSMYASLGERVSITCRASQDIYSYLSWFQQKPGKSPKTLIYRANRLVDGVPSRFS |
| (SEQ ID | | GSGSGQDYSLTISSLEYEDMGIYYCLQYDEFPYTFGGGTKLEIK (SEQ ID NO: 189) |
| NO: 273) | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

### Example 9. Binding Activity of Bispecific and Bi-Paratopic ROR1×4-1BB Antibodies

### 9.1 Chimeric Antibodies

### Binding Affinity

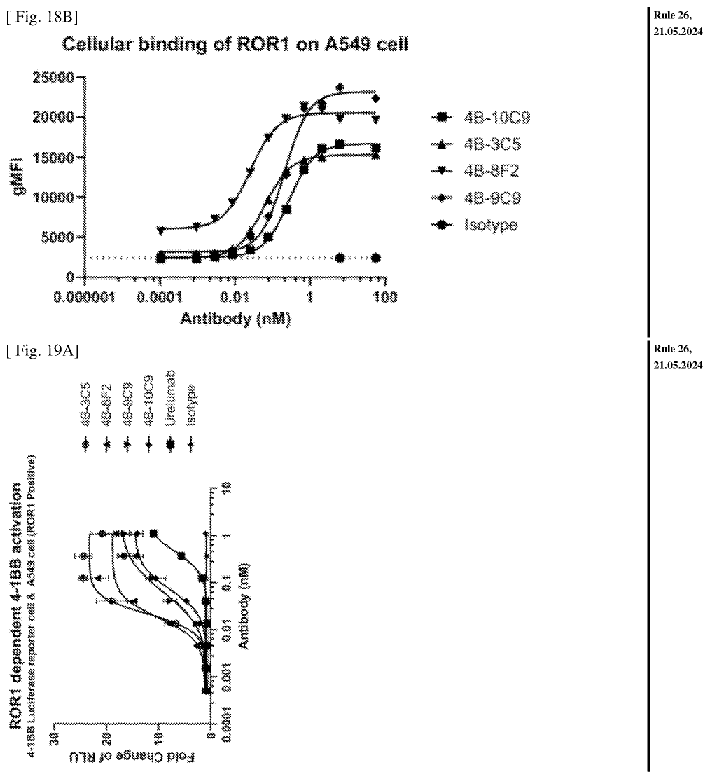

FACS binding activity shows that ROR1×4-1BB BsAbs binding with ROR1 endogenous expressed tumor cell A549 and 4-1BB overexpressed HEK293 cell in a dose-dependent manner (FIGS. 18A-18B).

### Epitope Binning

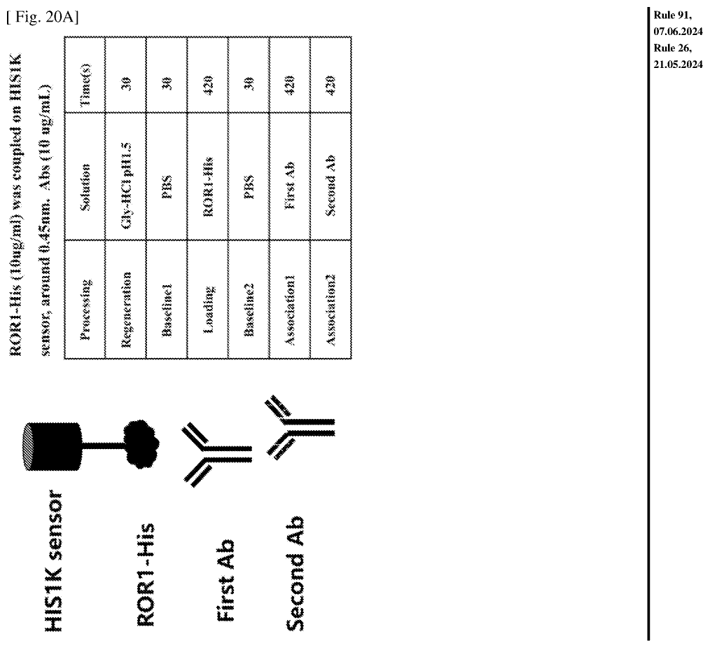

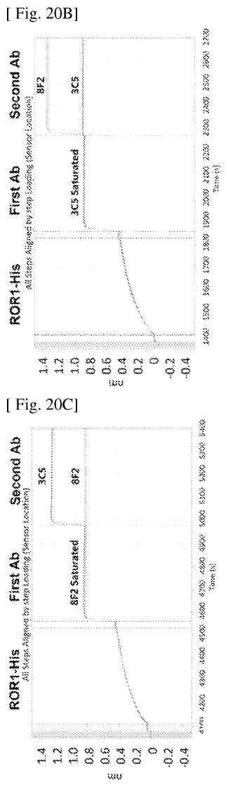

In an epitope binning of the anti-ROR1 mAbs, 3C5 and 8F2 showed non-competitive or non-overlapping binding epitope with ROR1 antigen, as detected by Octet (see FIGS. 20A-20C).

Based on the non-overlapping binding epitopes of 3C5 and 8F2 with ROR1, bi-paratopic ROR1×4-1BB antibodies (Table 6A) were designed on the hypothesis that bi-paratopic antigen targeting antibody could make more 4-1BB clustering and induce stronger 4-1BB pathway activation.

### Reporter Gene Assay

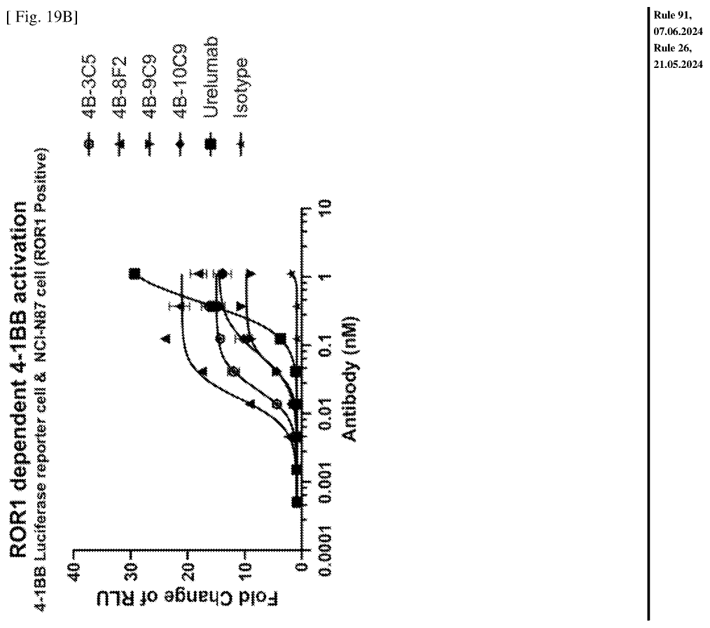

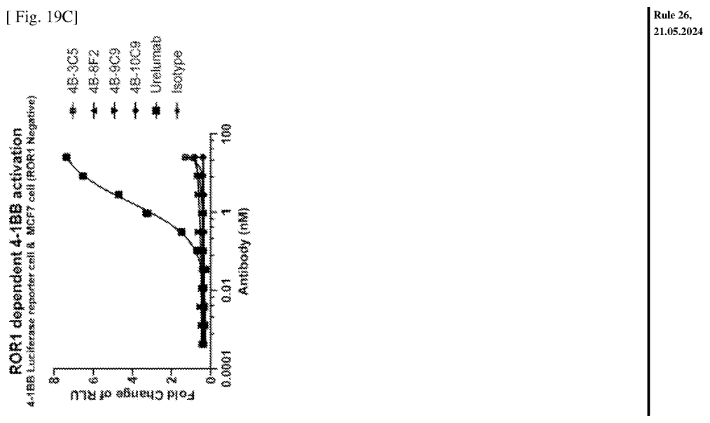

In this assay, Jurkat cell co-expressed 4-1BB and NFκB-Luciferase reporter were used as reporter cell line, ROR1 positive or negative cell lines were used as target cells. Report cells and target cells were co-cultured with gradient-diluted ROR1×4-1BB BsAbs or anti-4-1BB Urelumab for 6 hours, then One-Glo™ Reagent was used to readout the 4-1BB NFκB activation signal. Here, 4B-3C5 and 4B-8F2 BsAbs displayed strong ROR1 dependent 4-1BB activation (FIGS. 19A-19C).

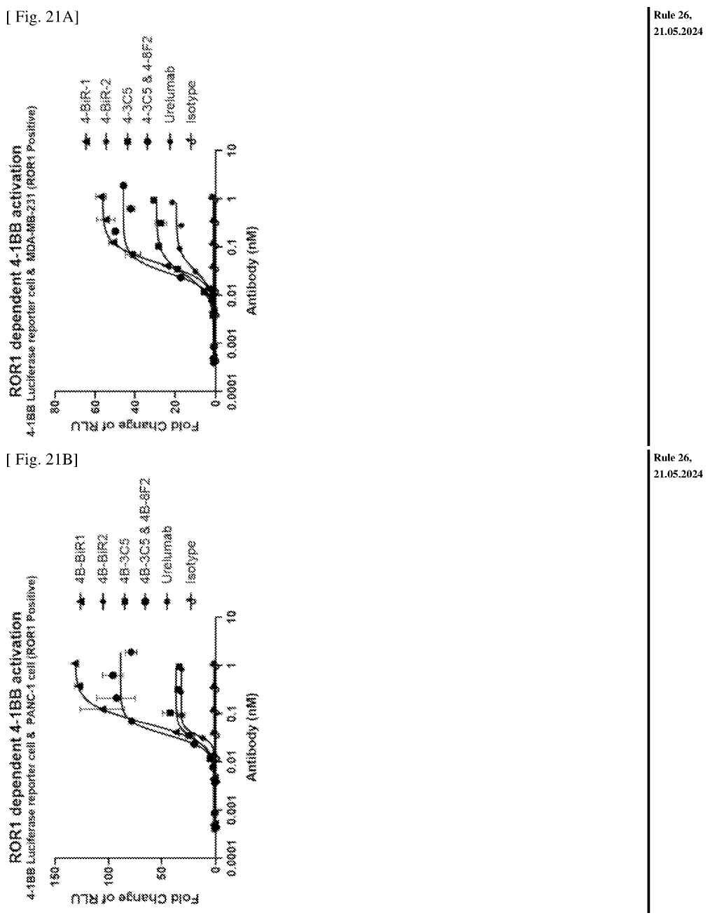

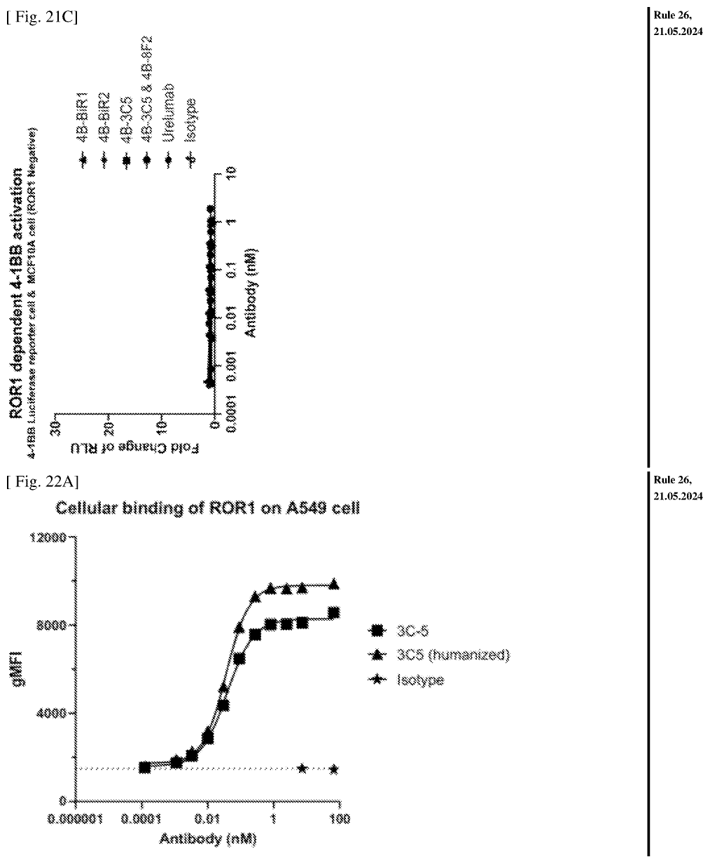

A similar reporter gene assay was performed using the bi-paratopic ROR1×4-1BB antibodies. The result shows that 4-BiR1 format bi-paratopic ROR1×4-1BB TsAb shows the best 4-1BB activation (see FIGS. 21A-21C).

### 9.2 Humanized Antibodies

Humanized anti-ROR1 antibodies were used to design mono-topic (bispecific) and bi-paratopic ROR1×4-1BB TsAb (see Table 6B).

TABLE 6B
Humanized bispecific and bi-paratopic antibodies

| | | |
|---|---|---|
| Chain | Domain | Amino Acid sequence |

4B-3C5 (humanized) or 4B-h3C5
humanized 3C5 IgG x 4-1BB VHH

| | | |
|---|---|---|
| HC | Humanized 3C5 | EVQLVESGGGLVKPGGSLRLSCAASGFTFSSYAMSWVRQAPGKRLEWVSSISSGGTTYYP |
| (SEQ ID | VH | DTVKGRFTISRDNAKNSLYLQMNSLRAEDTAVYYCARDALYYGSSLYYAMDYWGQGTLVT |
| NO: 274) | | VSS (SEQ ID NO: 222) |
| | IgG (CH) | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSY |
| | | ADSVKGRFTISRDNAKNTLYLQMNSLRAEDTAVYYCAADLLRAGTPLSSYEFNYWGQGTL |
| | | VTVSS (SEQ ID NO: 27) |
| LC | Humanized 3C5- | DIQMTQSPSSLSASVGDRVTITCRASQDIYSYLSWFQQKPGKAPKTLIYRANRLVDAVPS |
| (SEQ ID | VL | RFSGSGSGQDYTLTISSLQPEDFATYYCLQYDEFPYTFGQGTKLEIK (SEQ ID NO: |
| NO: 275) | | 223) |
| | IgG (CL) | RTVAAPSVFIFPPSDEQLKSGTASVVCLLNNFYPREAKVOWKVDNALQSGNSQESVTEQD |
| | | SKDSTYSLSSTLTLSKADYEKHKVYACEVTHQGLSSPVTKSENRGEC (SEQ ID NO: |
| | | 258) |

4B-8F2 (humanized) or 4B-h8F2
humanized 8F2 IgG x 4-1BB VHH

| | | |
|---|---|---|
| HC | Humanized 8F2 | QVQLVQSGAEVKKPGASVKVSCKASGYTFTTYVMHWVRQAPGQGLEWMGYINPYNGGIRY |
| (SEQ ID | VH | NEKFKGRVTMTRDTSTSTVYMELSSLRSEDTAVYYCARRERGVYYGMDEWGQGTLVTVSS |
| NO: 276) | | (SEQ ID NO: 224) |
| | IgG (CH) | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKENWYVDGVEVHNAKTKPREEQYN |
| | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSY |
| | | ADSVKGRFTISRDNAKNTLYLQMNSLRAEDTAVYYCAADLLRAGTPLSSYEFNYWGQGTL |
| | | VTVSS (SEQ ID NO: 27) |
| LC | Humanized 8F2- | DIVMTQTPLSLSVTPGQPASISCKSSQSLLHSNGKTYLNWYLQKPGQSPQLLIYLVSKLE |
| (SEQ ID | VL | SGVPDRFSGSGSGTDFTLKISRVEAEDVGVYYCLQATYFPYTFGQGTKLEIK (SEQ ID |
| NO: 277) | | NO: 225) |
| | IgG (CL) | RTVAAPSVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSENRGEC (SEQ ID NO: |
| | | 258) |

4B-BiR1(Humanized) or 4B-hBiR1: 4B-BiR1:
CH1/CL (h3C5 IgG x 4-1BB VHH) + CH2 (h8F2 scFv-IgG′(CH)-4-1BB VHH)

| | | |
|---|---|---|
| HC1 | Humanized 3C5 | EVQLVESGGGLVKPGGSLRLSCAASGFTFSSYAMSWVRQAPGKRLEWVSSISSGGTTYYP |
| (SEQ ID | VH.SA | DTVKGRFTISRDNAKNSLYLQMNSLRAEDTAVYYCARDALYYGSSLYYAMDYWGQGTLVT |
| NO: 278) | | VSS (SEQ ID NO: 222) |
| | IgG (CH) | ASTKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQYN |
| | | STYRVVSVLTVLHQDWLNGKEYKCKVSNKALPAPIEKTISKAKGQPREPQVYTLPPCREE |
| | | MTKNQVSLWCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO: 253) |
| | Linker | GGGGGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSY |
| | | ADSVKGRFTISRDNAKNTLYLQMNSLRAEDTAVYYCAADLLRAGTPLSSYEFNYWGQGTL |
| | | VTVSS (SEQ ID NO: 27) |
| HC2 | Humanized 8F2 | DIVMTQTPLSLSVTPGQPASISCKSSQSLLHSNGKTYLNWYLQKPGQSPQLLIYLVSKLE |
| (SEQ ID | scFv (VL-VH) | SGVPDRFSGSGSGTDFTLKISRVEAEDVGVYYCLQATYFPYTFGQGTKLEIK (SEQ ID |
| NO: 279) | | NO: 225) |
| | | GGGGSGGGGSGGGGS (SEQ ID NO: 23) |
| | | QVQLVQSGAEVKKPGASVKVSCKASGYTFTTYVMHWVRQAPGQGLEWMGYINPYNGGIRY |
| | | NEKFKGRVTMTRDTSTSTVYMELSSLRSEDTAVYYCARRERGVYYGMDEWGQGTLVTVSS |
| | | (SEQ ID NO: 224) |
| | IgG′ (CH) | AAEPKSSDKTHTCPPCPAPELLGGPSVFLFPPKPKDTLMISRTPEVTCVVVDVSHEDPEV |
| | | KFNWYVDGVEVHNAKTKPREEQYNSTYRVVSVLTVLHQDWLNGKEYKCKVSNKALPAPIE |
| | | KTISKAKGQPREPQVCTLPPSREEMTKNQVSLSCAVKGFYPSDIAVEWESNGQPENNYKT |
| | | TPPVLDSDGSFFLVSKLTVDKSRWQQGNVFSCSVMHEALHNHYTQKSLSLSPGK (SEQ |
| | | ID NO: 255) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSY |
| | | ADSVKGRFTISRDNAKNTLYLQMNSLRAEDTAVYYCAADLLRAGTPLSSYEFNYWGQGTL |
| | | VTVSS (SEQ ID NO: 27) |
| LC | Humanized 3C5 | DIQMTQSPSSLSASVGDRVTITCRASQDIYSYLSWFQQKPGKAPKTLIYRANRLVDAVPS |
| (SEQ ID | VL.GA | RFSGSGSGQDYTLTISSLQPEDFATYYCLQYDEFPYTFGQGTKLEIK (SEQ ID NO: |
| NO: 280) | | 223) |
| | IgG (CL) | RTVAAPSVFIFPPSDEQLKSGTASVVCLLNNFYPREAKVQWKVDNALOSGNSQESVTEQD |
| | | SKDSTYSLSSTLTLSKADYEKHKVYACEVTHQGLSSPVTKSENRGEC (SEQ ID NO: |
| | | 258) |

### Cellular Binding Affinity and Reporter Gene Assay

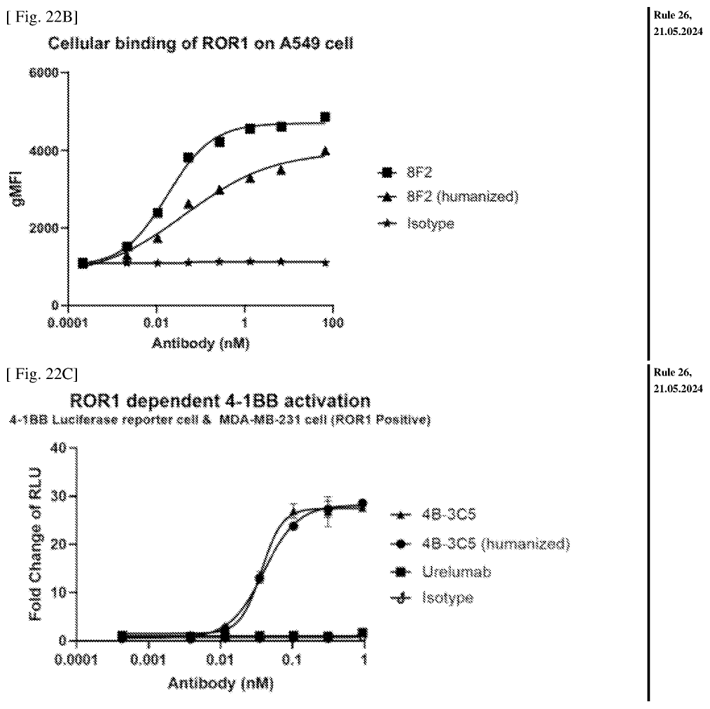

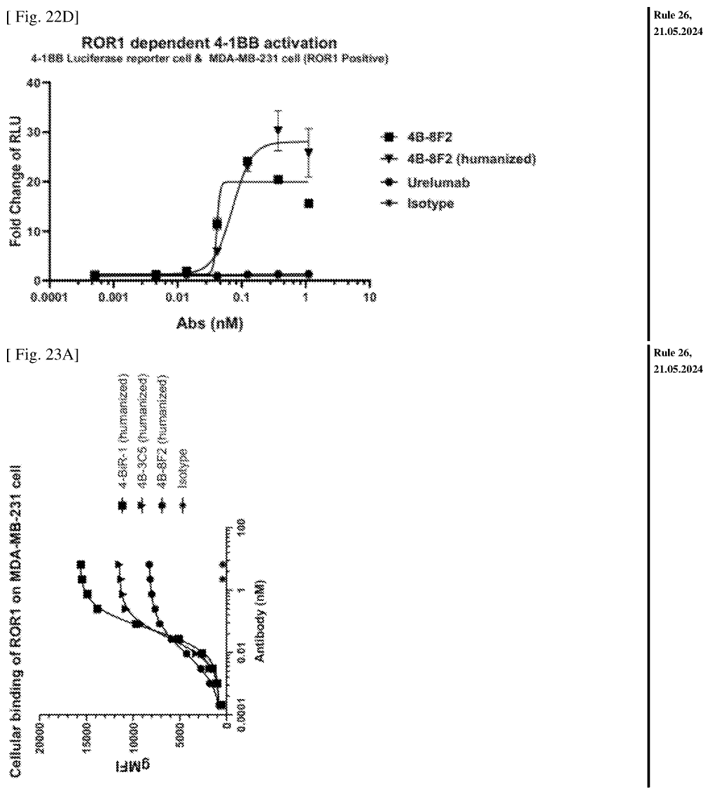

The cellular binding of humanized anti-ROR1 mAbs as measured by FACS, and ROR1 dependent 4-1BB activation of humanized ROR1×4-1BB BsAbs were tested similarly as above. The humanized mAbs showed better or comparable cellular binding (FIGS. 22A-22B), and the humanized BsAbs showed better or comparable ROR1 dependent 4-1BB activation as compared to the chimeric antibodies (FIGS. 22C-22D), while the 4-1BB VHH control urelumab does not activate 4-1BB in the ROR1 positive tumor cell lines in the corresponding concentration range.

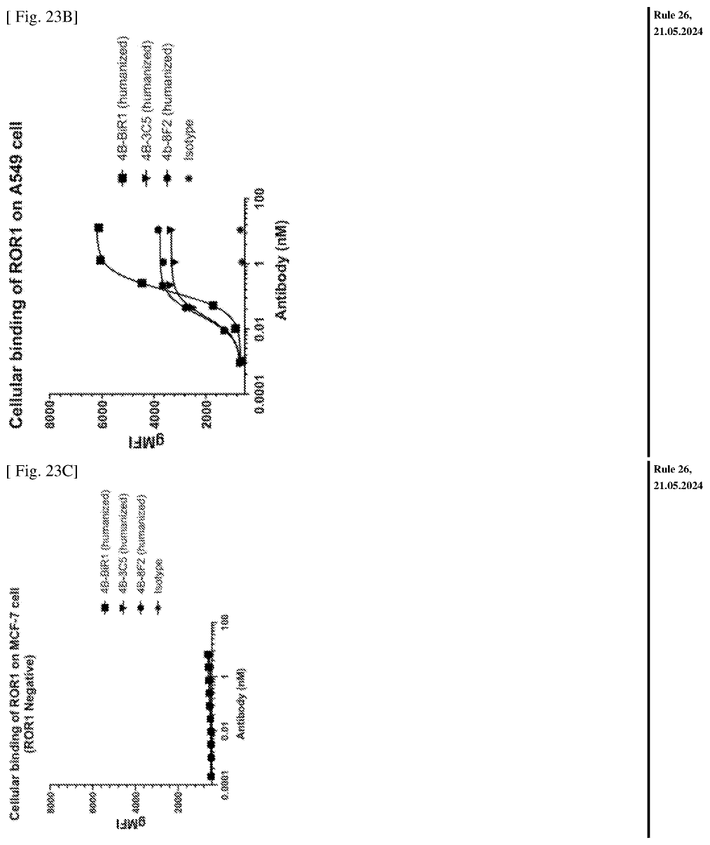

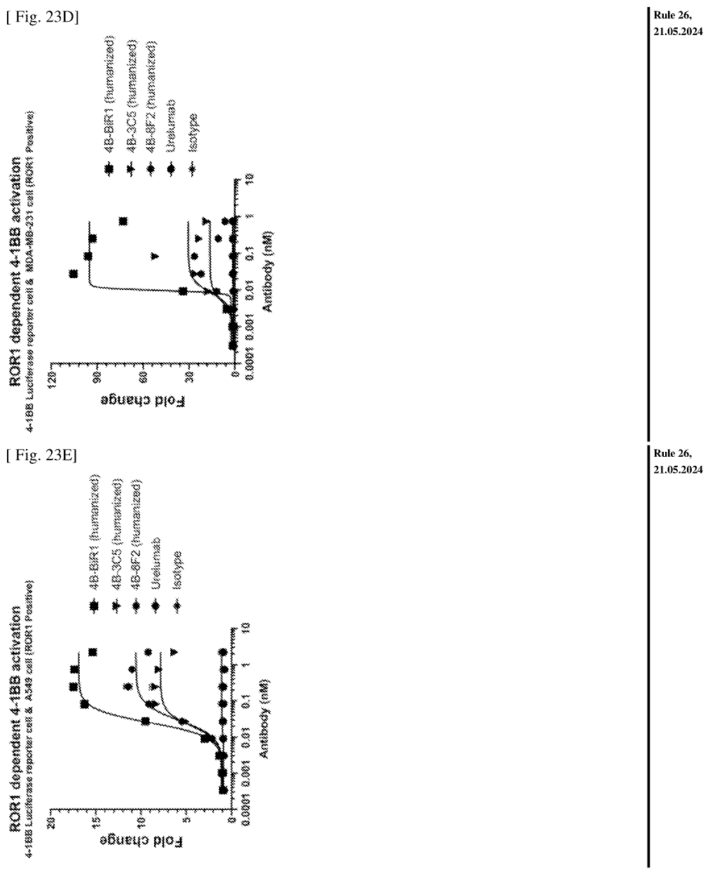

The comparison between the humanized antibodies showed that the bi-paratopic antibody 4-BiR1 has higher cellular binding of ROR1 in the ROR1-positive tumor cell lines (FIGS. 23A-23C) and higher ROR1 dependent 4-1BB activation (FIGS. 23D-23F), as compared with 4B-3C5 and 4B-8F5, while 4B-3C5 and 4B-8F5 showed similar cellular binding affinity and ROR1 dependent 4-1BB activation. There is no ROR1 independent 4-1BB activation for all the tested antibodies in the ROR1 negative tumor cell line.

### SPR

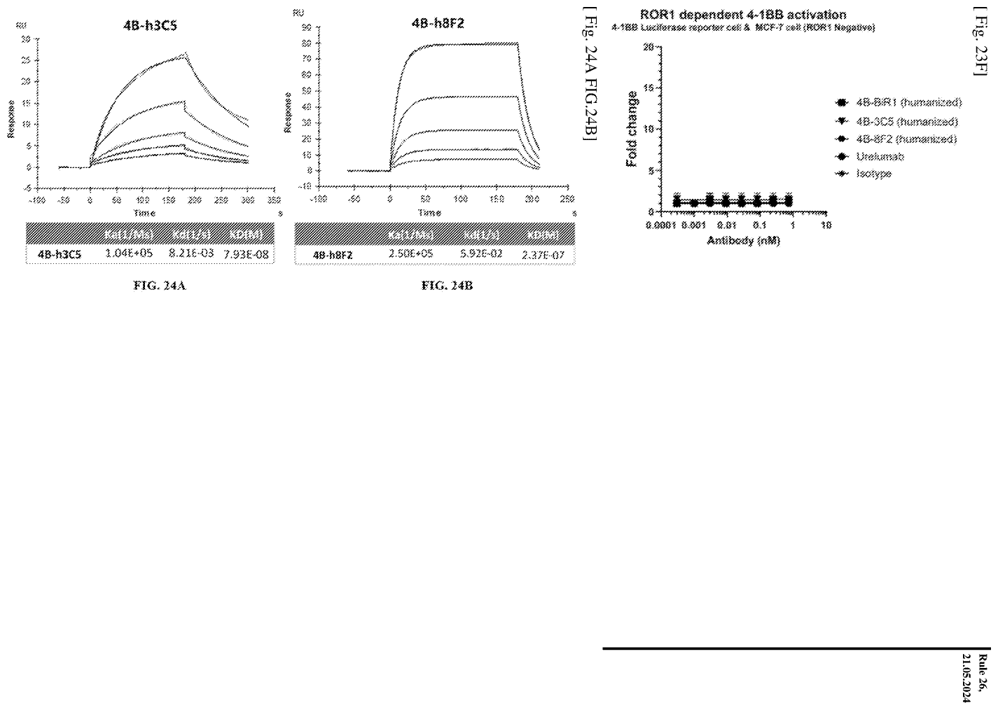

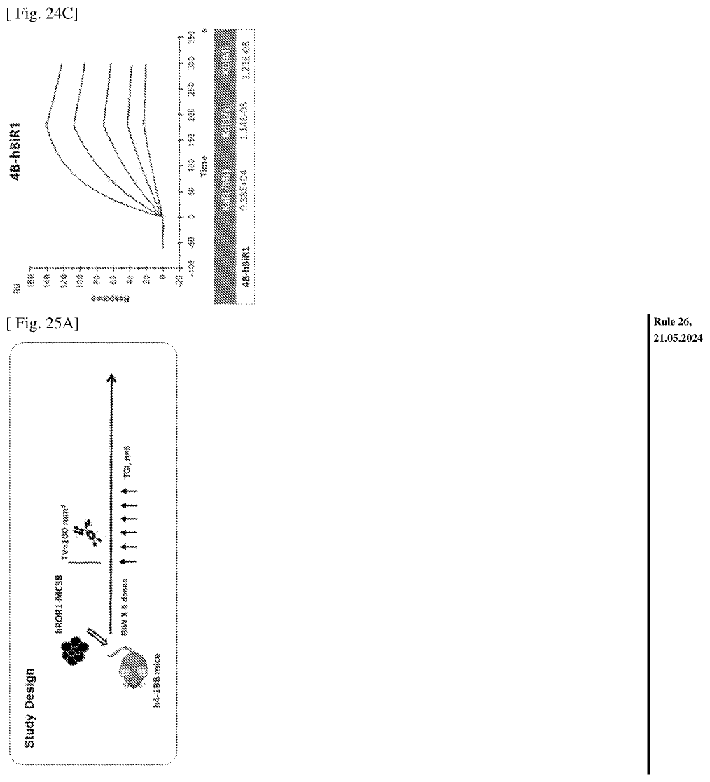

A surface plasmon resonance assay showed the binding affinity to ROR1 of the humanized antibodies 4B-h3C5 with a K_{D} value of 7.93E-08 M, 4B-h8F5 with a K_{D} value of 2.37E-07 M, and a 4B-hBiR1 with a K_{D} value of 1.21E-08 M (FIGS. 24A-24C).

### In Vivo Efficacy

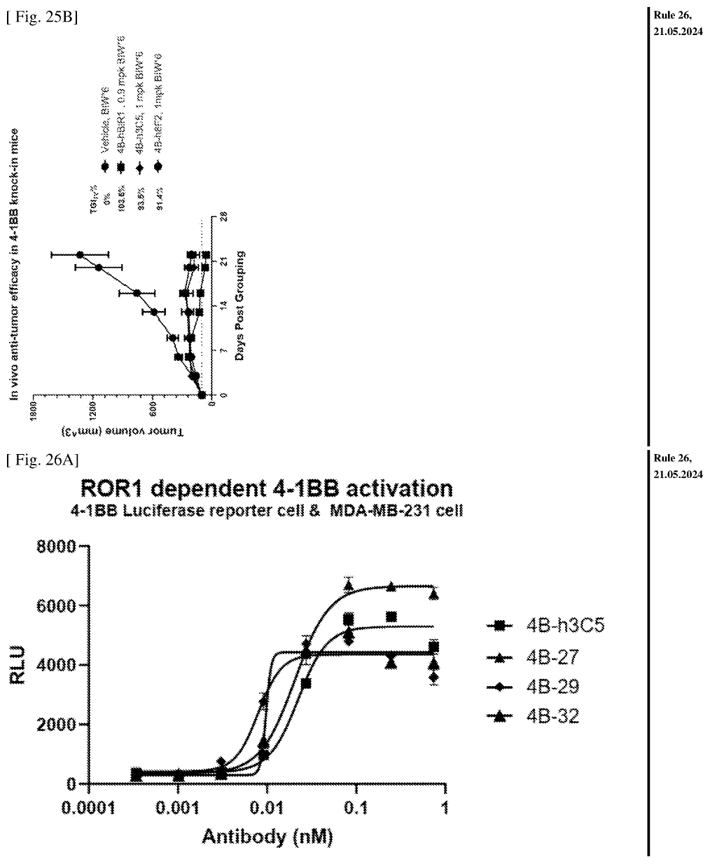

A syngeneic model (4-1BB humanized mice (Biocytogen)) was used to evaluate the in vivo efficacy of mono-topic and bi-paratopic ROR1×4-1BBs inoculated with the ROR1-MC38 cell line. All the tested humanized antibodies showed significant inhibition to the tumor growth (FIGS. 25A-25B).

### 9.3 Antibody Affinity Maturation

Considering the low yield of 4-hiBiR1, potential PTM in the anti-ROR1 sequences and low affinity of h3C5 and h8F2, the antibodies were engineered with affinity maturation (Table 6C and 6D).

TABLE 6C
Designed bispecific antibody sequences (affinity matured)

| | | |
|---|---|---|
| Chain | Domain | Amino Acid sequence |

4B-3C5-27
3C5-27 IgG x 4-1BB VHH

| | | |
|---|---|---|
| HC | 3C5-27 VH | EVQLVESGGGLVKPGGSLRLSCAASGFTFSSYAMSWVRQAPGKRLEWVSSISSGGTTYYPDTV |
| (SEQ ID | | KGRFTISRDNAKNSLYLQMNSLRAEDTAVYYCARDALYYGGSLYYAMDYWGQGTLVTVSS |
| NO: 281) | | (SEQ ID NO: 226) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5-27 VL | DIQMTQSPSSLSASVGDRVTITCRASQDIYSYLSWFQQKPGKAPKTLIYRANRLVDAVPSRFS |
| (SEQ ID | | GSGSGQDYTLTISSLQPEDFATYYCLQYDEFPYTFGQGTKLEIK (SEQ ID NO: 223) |
| NO: 282) | | |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-3C5-29
3C5-29 IgG x 4-1BB VHH

| | | |
|---|---|---|
| HC | 3C5-29 VH | EVQLVESGGGLVKPGGSLRLSCAASGFTFSRYAMSWVRQAPGKRLEWVSSISSGGNTYYPDTV |
| (SEQ ID | | KGRFTISRDNAKNSLYLQMNSLRAEDTAVYYCARDALYYGSSLYYAMDYWGQGTLVTVSS |
| NO: 110) | | (SEQ ID NO: 228) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLE |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5-29 VL | DIQMTQSPSSLSASVGDRVTITCRASQDIYSYLSWFQQKPGKAPKTLIYRENRLVDAVPSRFS |
| (SEQ ID | | GSGSGQDYTLTISSLQPEDFATYYCLQYDEFPYTFGQGTKLEIK (SEQ ID NO: 229) |
| NO: 111) | | |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-3C5-32
3C5-32 IgG x 4-1BB VHH

| | | |
|---|---|---|
| HC | 3C5-32 VH | EVQLVESGGGLVKPGGSLRLSCAASGFTFRSYAMSWVRQAPGKRLEWVSSISSGGTRYYPDTV |
| (SEQ ID | | KGRFTISRDNAKNSLYLQMNSLRAEDTAVYYCARDALYYGSSLYYAMDYWGQGTLVTVSS |
| NO: 112) | | (SEQ ID NO: 234) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 3C5-32 VL | DIQMTQSPSSLSASVGDRVTITCRASQDIYSYLSWFQQKPGKAPKTLIYRENRLVDAVPSRES |
| (SEQ ID | | GSGSGQDYTLTISSLQPEDFATYYCLQYDEFPYTFGQGTKLEIK (SEQ ID NO: 229) |
| NO: 113) | | |
| | IgG (CL) | RTVAAPSVFIFPPSDEQLKSGTASVVCLLNNFYPREAKVOWKVDNALQSGNSQESVTEQDSKD |
| | | STYSLSSTLTLSKADYEKHKVYACEVTHQGLSSPVTKSENRGEC (SEQ ID NO: 258) |

4B-8F2-73
8F2-73 IgG x 4-1BB VHH

| | | |
|---|---|---|
| HC | 8F2-73 VH | QVQLVQSGAEVKKPGASVKVSCKASGYRFTTYVMHWVRQAPGQGLEWMGYINPYNGVIRYNEK |
| (SEQ ID | | FKGRVTMTRDTSTSTVYMELSSLRSEDTAVYYCARRERGVYYGMDEWGQGTLVTVSS (SEQ |
| NO: 114) | | ID NO: 235) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2-73 VL | DIVMTQTPLSLSVTPGQPASISCKSSQSLLHSNGKTYLNWYLQKPGQSPQLLIYLVSKLSSGV |
| (SEQ ID | | PDRFSGSGSGTDFTLKISRVEAEDVGVYYCLQATYFPYTFGQGTKLEIK (SEQ ID NO: |
| NO: 115) | | 236) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-8F2-87
8F2-87 IgG x 4-1BB VHH

| | | |
|---|---|---|
| HC | 8F2-87 VH | QVQLVQSGAEVKKPGASVKVSCKASGYTFTTYVMHWVRQAPGQGLEWMGYINPYNGGIRYNEK |
| (SEQ ID | | FKGRVTMTRDTSTSTVYMELSSLRSEDTAVYYCARRERGVYYGMSEWGQGTLVTVSS (SEQ |
| NO: 116) | | ID NO: 240) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2-87 VL | DIVMTQTPLSLSVTPGQPASISCKSSQSLLHSNGKTYLNWYLQKPGQSPQLLIYLVSKLESGV |
| (SEQ ID | | PDRFSGSGSGTDFTLKISRVEAEDVGVYYCYQATYFPYTFGQGTKLEIK (SEQ ID NO: |
| NO: 117) | | 241) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-8F2-87DS
8F2-87DS IgG x 4-1BB VHH

| | | |
|---|---|---|
| HC | 8F2-87DS VH | QVQLVQSGAEVKKPGASVKVSCKASGYTFTTYVMHWVRQAPGQGLEWMGYINPYSGGIRYNEK |
| (SEQ ID | | FKGRVTMTRDTSTSTVYMELSSLRSEDTAVYYCARRERGVYYGMSEWGQGTLVTVSS (SEQ |
| NO: 118) | | ID NO: 244) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2-87DS VL | DIVMTQTPLSLSVTPGQPASISCKSSQSLLHSNDKTYLNWYLQKPGQSPQLLIYLVSKLESGV |
| (SEQ ID | | PDRFSGSGSGTDFTLKISRVEAEDVGVYYCYQATYFPYTFGQGTKLEIK (SEQ ID NO: |
| NO: 119) | | 245) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-8F2-82
8F2-82 IgG x 4-1BB VHH

| | | |
|---|---|---|
| HC | 8F2-82 VH | QVQLVQSGAEVKKPGASVKVSCKASGYTFTTYVMHWVRQAPGQGLEWMGYINPYSGGIRYNEK |
| (SEQ ID | | FKGRVTMTRDTSTSTVYMELSSLRSEDTAVYYCARRERGVTAGMDEWGQGTLVTVSS (SEQ |
| NO: 65) | | ID NO: 248) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2-82 VL | DIVMTQTPLSLSVTPGQPASISCKSSQSLLHSNGKTYLNWYLQKPGQSPQLLIYLVSKLESGV |
| (SEQ ID | | PDRFSGSGSGTDFTLKISRVEAEDVGVYYCLQATYFPYTFGQGTKLEIK (SEQ ID NO: |
| NO: 66) | | 225) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSENRGEC (SEQ ID NO: 258) |

4B-8F2-97
8F2-97 IgG x 4-1BB VHH

| | | |
|---|---|---|
| HC | 8F2-97 VH | QVQLVQSGAEVKKPGASVKVSCKASGYTFTNYVMHWVRQAPGQGLEWMGYINPYSGGIRYNEK |
| (SEQ ID | | FKGRVTMTRDTSTSTVYMELSSLRSEDTAVYYCARRERGVYYGMDEWGQGTLVTVSS (SEQ |
| NO: 68) | | ID NO: 250) |
| | IgG (CH) | ASTKGPSVFPLAPSSKSTSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREEQYNSTYRVVSVL |
| | | TVLHQDWLNGKEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNVFSCSVMHEAL |
| | | HNHYTQKSLSLSPGK (SEQ ID NO: 252) |
| | Linker | GGGGGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADS |
| | | VKGRFTISRDNAKNTLYLQMNSLRAEDTAVYYCAADLLRAGTPLSSYEFNYWGQGTLVTVSS |
| | | (SEQ ID NO: 27) |
| LC | 8F2-97 VL | DIVMTQTPLSLSVTPGQPASISCKSSQSLLHSNGKTYLNWYLQKPGQSPQLLIYLVSKLESGV |
| (SEQ ID | | PDRFSGSGSGTDFTLKISRVEAEDVGVYYCLQATYFPYTFGQGTKLEIK (SEQ ID NO: |
| NO: 69) | | 225) |
| | IgG (CL) | RTVAAPSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC (SEQ ID NO: 258) |

TABLE 6D
Designed bi-paratopic antibody sequences (affinity matured)

| | | |
|---|---|---|
| Chain | Domain | Amino Acid sequence |

4B-2987
CH1/CL (3C5-29 IgG x 4-1BB VHH) + CH2 (8F2-87 scFv-IgG′(CH)-4-1BB VHH)

| | | |
|---|---|---|
| HC1 | 3C5-29 VH | EVQLVESGGGLVKPGGSLRLSCAASGFTFSRYAMSWVRQAPGKRLEWVSSISSGGNTYYPDTVK |
| (SEQ ID | | GRFTISRDNAKNSLYLQMNSLRAEDTAVYYCARDALYYGSSLYYAMDYWGQGTLVTVSS (SEQ |
| NO: 73) | | ID NO: 228) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPOVYTLPPCREEMTKNQVSLWCLVKGFY |
| | | PSDIAVEWESNGQPENNYKTTPPVLDSDGSFFLYSKLTVDKSRWQQGNVFSCSVMHEALHNHYT |
| | | QKSLSLSPGK (SEQ ID NO: 253) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-87 scFv | DIVMTQTPLSLSVTPGQPASISCKSSQSLLHSNGKTYLNWYLQKPGQSPQLLIYLVSKLESGVP |
| (SEQ ID | (VL-VH) | DRFSGSGSGTDFTLKISRVEAEDVGVYYCYQATYFPYTFGQGTKLEIK (SEQ ID NO: |
| NO: 74) | | 241) |
| | | GGGGSGGGGSGGGGS (SEQ ID NO: 23) |
| | | QVQLVQSGAEVKKPGASVKVSCKASGYTFTTYVMHWVRQAPGQGLEWMGYINPYNGGIRYNEKF |
| | | KGRVTMTRDTSTSTVYMELSSLRSEDTAVYYCARRERGVYYGMSEWGQGTLVTVSS (SEQ ID |
| | | NO: 240) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGK (SEQ ID NO: 255) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-29 VL | DIQMTQSPSSLSASVGDRVTITCRASQDIYSYLSWFQQKPGKAPKTLIYRENRLVDAVPSRFSG |
| (SEQ ID | | SGSGQDYTLTISSLQPEDFATYYCLQYDEFPYTFGQGTKLEIK (SEQ ID NO: 229) |
| NO: 81) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

4B-3287
CH1/CL (3C5-32 IgG x 4-1BB VHH) + CH2 (8F2-87 scFv-IgG′(CH)-4-1BB VHH)

| | | |
|---|---|---|
| HC1 | 3C5-32 VH | EVQLVESGGGLVKPGGSLRLSCAASGFTFRSYAMSWVRQAPGKRLEWVSSISSGGTRYYPDTVK |
| (SEQ ID | | GRFTISRDNAKNSLYLQMNSLRAEDTAVYYCARDALYYGSSLYYAMDYWGQGTLVTVSS (SEQ |
| NO: 82) | | ID NO: 234) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVMHEALHNHYT |
| | | QKSLSLSPGK (SEQ ID NO: 253) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-87 scFv | DIVMTQTPLSLSVTPGQPASISCKSSQSLLHSNGKTYLNWYLQKPGQSPQLLIYLVSKLESGVP |
| (SEQ ID | (VL-VH) | DRFSGSGSGTDFTLKISRVEAEDVGVYYCYQATYFPYTFGQGTKLEIK (SEQ ID NO: |
| NO: 89) | | 241) |
| | | GGGGSGGGGSGGGGS (SEQ ID NO: 23) |
| | | QVQLVQSGAEVKKPGASVKVSCKASGYTFTTYVMHWVRQAPGQGLEWMGYINPYNGGIRYNEKF |
| | | KGRVTMTRDTSTSTVYMELSSLRSEDTAVYYCARRERGVYYGMSEWGQGTLVTVSS (SEQ ID |
| | | NO: 240) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGK (SEQ ID NO: 255) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-32 VL | DIQMTQSPSSLSASVGDRVTITCRASQDIYSYLSWFQQKPGKAPKTLIYRENRLVDAVPSRESG |
| (SEQ ID | | SGSGQDYTLTISSLQPEDFATYYCLQYDEFPYTFGQGTKLEIK (SEQ ID NO: 229) |
| NO: 90) | | |
| | IgG (CL) | RTVAAPSVFIFPPSDEQLKSGTASVVCLLNNFYPREAKVOWKVDNALQSGNSQESVTEQDSKDS |
| | | TYSLSSTLTLSKADYEKHKVYACEVTHQGLSSPVTKSENRGEC (SEQ ID NO: 258) |

4B-2973
CH1/CL (3C5-29 IgG x 4-1BB VHH) + CH2 (8F2-73 scFv-IgG′(CH)-4-1BB VHH)

| | | |
|---|---|---|
| HC1 | 3C5-29 VH | EVQLVESGGGLVKPGGSLRLSCAASGFTFSRYAMSWVRQAPGKRLEWVSSISSGGNTYYPDTVK |
| (SEQ ID | | GRFTISRDNAKNSLYLQMNSLRAEDTAVYYCARDALYYGSSLYYAMDYWGQGTLVTVSS (SEQ |
| NO: 105) | | ID NO: 228) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVMHEALHNHYT |
| | | QKSLSLSPGK (SEQ ID NO: 253) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-73 scFv | DIVMTQTPLSLSVTPGQPASISCKSSQSLLHSNGKTYLNWYLQKPGQSPQLLIYLVSKLSSGVP |
| (SEQ ID | (VL-VH) | DRFSGSGSGTDFTLKISRVEAEDVGVYYCLQATYFPYTFGQGTKLEIK (SEQ ID NO: |
| NO: 109) | | 236) |
| | | GGGGSGGGGSGGGGS (SEQ ID NO: 23) |
| | | QVQLVQSGAEVKKPGASVKVSCKASGYRFTTYVMHWVRQAPGQGLEWMGYINPYNGVIRYNEKF |
| | | KGRVTMTRDTSTSTVYMELSSLRSEDTAVYYCARRERGVYYGMDEWGQGTLVTVSS (SEQ ID |
| | | NO: 235) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGK (SEQ ID NO: 255) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-29 VL | DIQMTQSPSSLSASVGDRVTITCRASQDIYSYLSWFQQKPGKAPKTLIYRENRLVDAVPSRESG |
| (SEQ ID | | SGSGQDYTLTISSLQPEDFATYYCLQYDEFPYTFGQGTKLEIK (SEQ ID NO: 229) |
| NO: 124) | | |
| | IgG (CL) | RTVAAPSVFIFPPSDEQLKSGTASVVCLLNNFYPREAKVOWKVDNALQSGNSQESVTEQDSKDS |
| | | TYSLSSTLTLSKADYEKHKVYACEVTHQGLSSPVTKSENRGEC (SEQ ID NO: 258) |

4B-3287
CH1/CL (3C5-32 IgG x 4-1BB VHH) + CH2 (8F2-87 seFv-IgG′(CH)-4-1BB VHH)

| | | |
|---|---|---|
| HC1 | 3C5-32 VH | EVQLVESGGGLVKPGGSLRLSCAASGFTFRSYAMSWVRQAPGKRLEWVSSISSGGTRYYPDTVK |
| (SEQ ID | | GRFTISRDNAKNSLYLQMNSLRAEDTAVYYCARDALYYGSSLYYAMDYWGQGTLVTVSS (SEQ |
| NO: 131) | | ID NO: 234) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPOVYTLPPCREEMTKNQVSLWCLVKGFY |
| | | PSDIAVEWESNGQPENNYKTTPPVLDSDGSFFLYSKLTVDKSRWQQGNVFSCSVLHEALHSHYT |
| | | QKSLSLSPGK (SEQ ID NO: 257) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-87 scFv | DIVMTQTPLSLSVTPGQPASISCKSSQSLLHSNDKTYLNWYLQKPGQSPQLLIYLVSKLESGVP |
| (SEQ ID | (VL-VH) | DRFSGSGSGTDFTLKISRVEAEDVGVYYCYQATYFPYTFGQGTKLEIK (SEQ ID NO: |
| NO: 132) | | 245) |
| | | GGGGSGGGGSGGGGS (SEQ ID NO: 23) |
| | | QVQLVQSGAEVKKPGASVKVSCKASGYTFTTYVMHWVRQAPGQGLEWMGYINPYSGGIRYNEKF |
| | | KGRVTMTRDTSTSTVYMELSSLRSEDTAVYYCARRERGVYYGMSEWGQGTLVTVSS (SEQ ID |
| | | NO: 244) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVLHEALHSHYTQKSLSLSPGK (SEQ ID NO: 256) |
| | Linker | GGGGSGGGGGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-32 VL | DIQMTQSPSSLSASVGDRVTITCRASQDIYSYLSWFQQKPGKAPKTLIYRENRLVDAVPSRESG |
| (SEQ ID | | SGSGQDYTLTISSLQPEDFATYYCLQYDEFPYTFGQGTKLEIK (SEQ ID NO: 229) |
| NO: 135) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

4B-2773
CH1/CL (3C5-32 IgG x 4-1BB VHH) + CH2 (8F2-87 scFv-IgG′(CH)-4-1BB VHH)

| | | |
|---|---|---|
| HC1 | 3C5-27 VH | EVQLVESGGGLVKPGGSLRLSCAASGFTFSSYAMSWVRQAPGKRLEWVSSISSGGTTYYPDTVK |
| (SEQ ID | | GRFTISRDNAKNSLYLQMNSLRAEDTAVYYCARDALYYGGSLYYAMDYWGQGTLVTVSS (SEQ |
| NO: 142) | | ID NO: 226) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVLHEALHSHYT |
| | | QKSLSLSPGK (SEQ ID NO: 257) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-73 scFv | DIVMTQTPLSLSVTPGQPASISCKSSQSLLHSNGKTYLNWYLQKPGQSPQLLIYLVSKLSSGVP |
| (SEQ ID | (VL-VH) | DRFSGSGSGTDFTLKISRVEAEDVGVYYCLQATYFPYTFGQGTKLEIK (SEQ ID NO: |
| NO: 143) | | 236) |
| | | GGGGSGGGGSGGGGS (SEQ ID NO: 23) |
| | | QVQLVQSGAEVKKPGASVKVSCKASGYRFTTYVMHWVRQAPGQGLEWMGYINPYNGVIRYNEKF |
| | | KGRVTMTRDTSTSTVYMELSSLRSEDTAVYYCARRERGVYYGMDEWGQGTLVTVSS (SEQ ID |
| | | NO: 235) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVLHEALHSHYTQKSLSLSPGK (SEQ ID NO: 256) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-27 VL | DIQMTQSPSSLSASVGDRVTITCRASQDIYSYLSWFQQKPGKAPKTLIYRANRLVDAVPSRESG |
| (SEQ ID | | SGSGQDYTLTISSLQPEDFATYYCLQYDEFPYTFGQGTKLEIK (SEQ ID NO: 223) |
| NO: 147) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

4B-2787DS-SS
CH1/CL (3C5-27 IgG x 4-1BB VHH) + CH2 (8F2-87DS/SS scFv-IgG′(CH)-4-1BB VHH)

| | | |
|---|---|---|
| HC1 | 3C5-27 VH | EVQLVESGGGLVKPGGSLRLSCAASGFTFSSYAMSWVRQAPGKRLEWVSSISSGGTTYYPDTVK |
| (SEQ ID | | GRFTISRDNAKNSLYLQMNSLRAEDTAVYYCARDALYYGGSLYYAMDYWGQGTLVTVSS (SEQ |
| NO: 148) | | ID NO: 226) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVLHEALHSHYT |
| | | QKSLSLSPGK (SEQ ID NO: 257) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-87DS/SS | DIVMTQTPLSLSVTPGQPASISCKSSQSLLHSNDKTYLNWYLQKPGQSPQLLIYLVSKLESGVP |
| (SEQ ID | scFv (VL-VH) | DRFSGSGSGTDFTLKISRVEAEDVGVYYCYQATYFPYTFGCGTKLEIK (SEQ ID NO: |
| NO: 283) | | 260) |
| | | GGGGSGGGGSGGGGS (SEQ ID NO: 23) |
| | | QVQLVQSGAEVKKPGASVKVSCKASGYTFTTYVMHWVRQAPGQCLEWMGYINPYSGGIRYNEKF |
| | | KGRVTMTRDTSTSTVYMELSSLRSEDTAVYYCARRERGVYYGMSEWGQGTLVTVSS (SEQ ID |
| | | NO: 259) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVLHEALHSHYTQKSLSLSPGK (SEQ ID NO: 256) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-27 VL | DIQMTQSPSSLSASVGDRVTITCRASQDIYSYLSWFQQKPGKAPKTLIYRANRLVDAVPSRFSG |
| (SEQ ID | | SGSGODYTLTISSLQPEDFATYYCLQYDEFPYTFGQGTKLEIK (SEQ ID NO: 223) |
| NO: 155) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

4B-2782
CH1/CL (3C5-27 IgG x 4-1BB VHH) +CH2 (8F2-82 seFv-IgG′(CH)-4-1BB VHH)

| | | |
|---|---|---|
| HC1 | 3C5-27 VH | EVQLVESGGGLVKPGGSLRLSCAASGFTFSSYAMSWVRQAPGKRLEWVSSISSGGTTYYPDTVK |
| (SEQ ID | | GRFTISRDNAKNSLYLQMNSLRAEDTAVYYCARDALYYGGSLYYAMDYWGQGTLVTVSS (SEQ |
| NO: 156) | | ID NO: 226) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVLHEALHSHYT |
| | | QKSLSLSPGK (SEQ ID NO: 257) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-82 scFv | DIVMTQTPLSLSVTPGQPASISCKSSQSLLHSNGKTYLNWYLQKPGQSPQLLIYLVSKLESGVP |
| (SEQ ID | (VL-VH) | DRFSGSGSGTDFTLKISRVEAEDVGVYYCLQATYFPYTFGQGTKLEIK (SEQ ID NO: |
| NO: 157) | | 225) |
| | | GGGGGGGGSGGGGS (SEQ ID NO: 23) |
| | | QVQLVQSGAEVKKPGASVKVSCKASGYTFTTYVMHWVRQAPGQGLEWMGYINPYSGGIRYNEKF |
| | | KGRVTMTRDTSTSTVYMELSSLRSEDTAVYYCARRERGVTAGMDEWGQGTLVTVSS (SEQ ID |
| | | NO: 248) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVLHEALHSHYTQKSLSLSPGK (SEQ ID NO: 256) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-27 VL | DIQMTQSPSSLSASVGDRVTITCRASQDIYSYLSWFQQKPGKAPKTLIYRANRLVDAVPSRFSG |
| (SEQ ID | | SGSGQDYTLTISSLQPEDFATYYCLQYDEFPYTFGQGTKLEIK (SEQ ID NO: 223) |
| NO: 163) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

4B-2797
CH1/CL (3C5-27 IgG x 4-1BB VHH) + CH2 (8F2-97 scFv-IgG′(CH)-4-1BB VHH)

| | | |
|---|---|---|
| HC1 | 3C5-27 VH | EVQLVESGGGLVKPGGSLRLSCAASGFTFSSYAMSWVRQAPGKRLEWVSSISSGGTTYYPDTVK |
| (SEQ ID | | GRFTISRDNAKNSLYLQMNSLRAEDTAVYYCARDALYYGGSLYYAMDYWGQGTLVTVSS (SEQ |
| NO: 164) | | ID NO: 226) |
| | IgG (CH) | ASTKGPSVFPLAPSSKSTSGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPCREEMTKNQVSLWCLVKGFY |
| | | PSDIAVEWESNGQPENNYKTTPPVLDSDGSFFLYSKLTVDKSRWQQGNVFSCSVLHEALHSHYT |
| | | QKSLSLSPGK (SEQ ID NO: 257) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| HC2 | 8F2-97 scFv | DIVMTQTPLSLSVTPGQPASISCKSSQSLLHSNGKTYLNWYLQKPGQSPQLLIYLVSKLESGVP |
| (SEQ ID | (VL-VH) | DRFSGSGSGTDFTLKISRVEAEDVGVYYCLQATYEPYTFGQGTKLEIK (SEQ ID NO: |
| NO: 218) | | 225) |
| | | GGGGSGGGGSGGGGS (SEQ ID NO: 23) |
| | | QVQLVQSGAEVKKPGASVKVSCKASGYTFTNYVMHWVRQAPGQGLEWMGYINPYSGGIRYNEKF |
| | | KGRVTMTRDTSTSTVYMELSSLRSEDTAVYYCARRERGVYYGMDEWGQGTLVTVSS (SEQ ID |
| | | NO: 250) |
| | IgG′ (CH) | AAEPKSSDKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEKTISKAKG |
| | | QPREPQVCTLPPSREEMTKNQVSLSCAVKGFYPSDIAVEWESNGQPENNYKTTPPVLDSDGSFF |
| | | LVSKLTVDKSRWQQGNVFSCSVLHEALHSHYTQKSLSLSPGK (SEQ ID NO: 256) |
| | Linker | GGGGSGGGGSGGGGS (SEQ ID NO: 23) |
| | 4-1BB VHH | QVQLQESGGGLVQPGGSLRLSCAASGYTYSSNCMGWFRQAPGKGLEGVAVICTGGGSPSYADSV |
| | | KGRFTISRDNAKNTLYLQMNSLRAEDTAVYYCAADLLRAGTPLSSYEFNYWGQGTLVTVSS |
| | | (SEQ ID NO: 27) |
| LC | 3C5-27 VL | DIQMTQSPSSLSASVGDRVTITCRASQDIYSYLSWFQQKPGKAPKTLIYRANRLVDAVPSRFSG |
| (SEQ ID | | SGSGODYTLTISSLQPEDFATYYCLQYDEFPYTFGQGTKLEIK (SEQ ID NO: 223) |
| NO: 237) | | |
| | IgG (CL) | RTVAAPSVFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSENRGEC (SEQ ID NO: 258) |

### Reporter Gene Assay

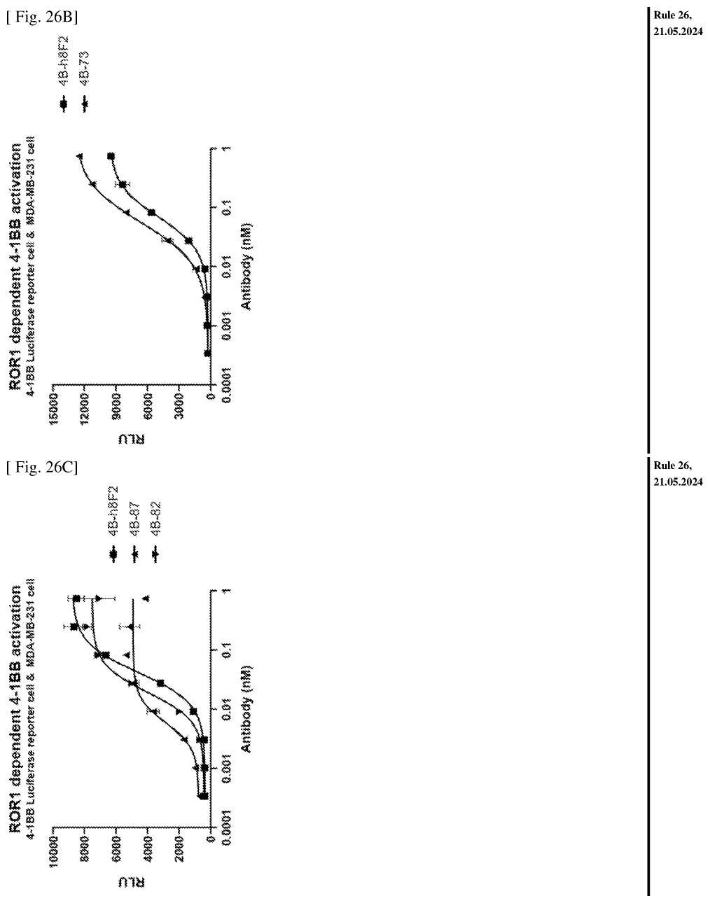

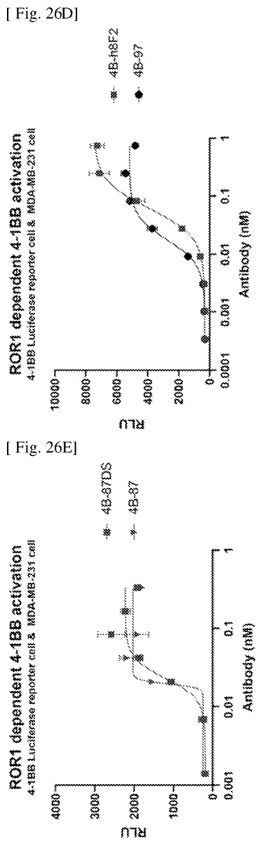

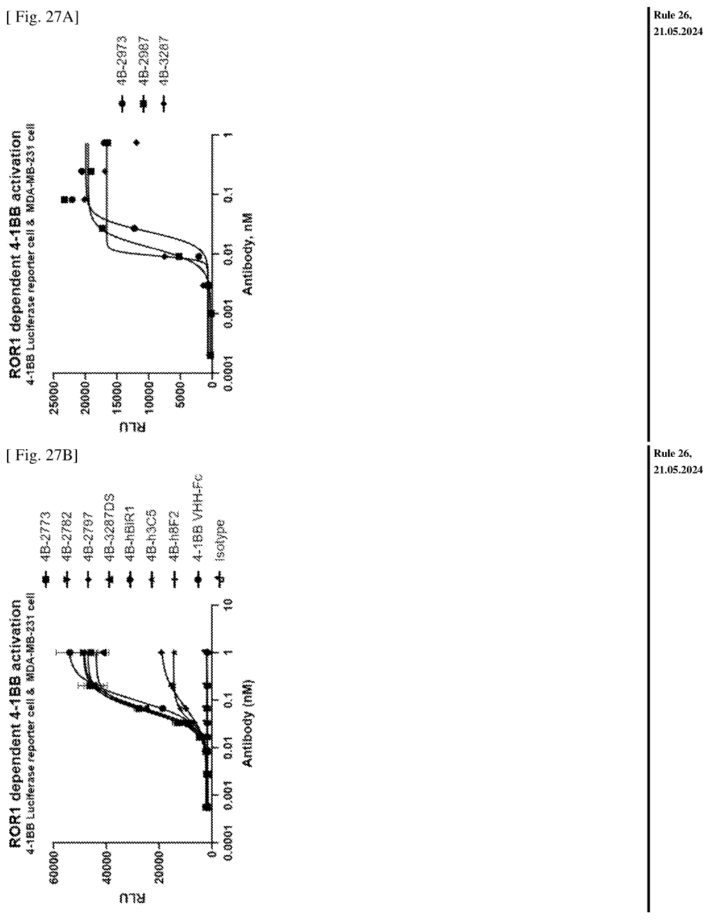

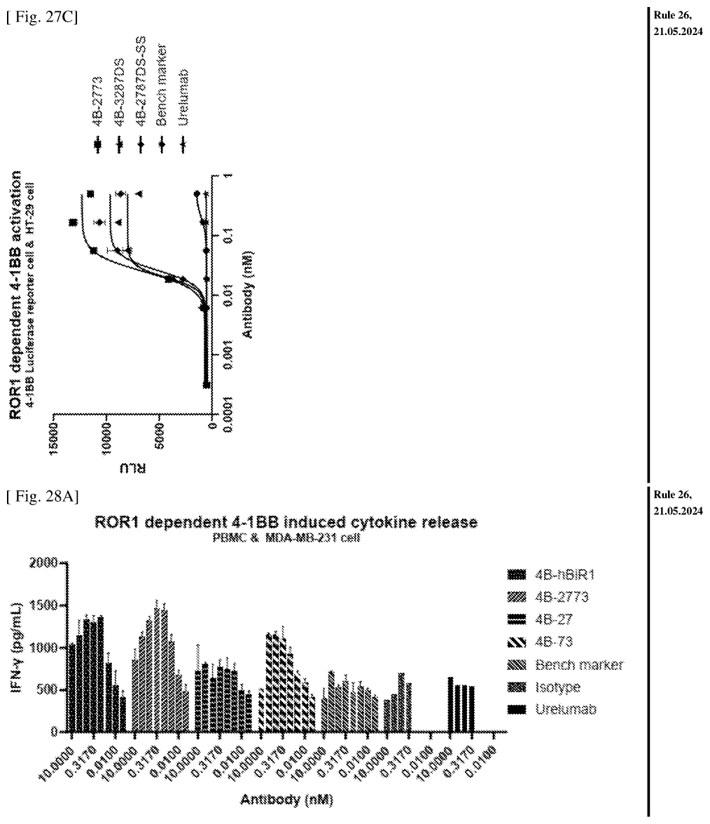

Similar reporter gene assay was performed using the affinity matured bispecfic or bi-paratopic ROR1×4-1BB antibodies. The results showed a better ROR1 dependent 4-1BB activation for the affinity matured bispecfic antibodies as compared to the humanized version (FIGS. 26A-26E). A much better ROR1 dependent 4-1BB activation was observed for the affinity matured bi-paratopic antibodies as compared to the humanized bispecific version and a benchmark ROR1×4-1BB BsAb from patent WO2021/101346A1 (Clone ID: BA6(NA)×1A10M12) (FIGS. 27A-27C).

### Cytokine Release Assay

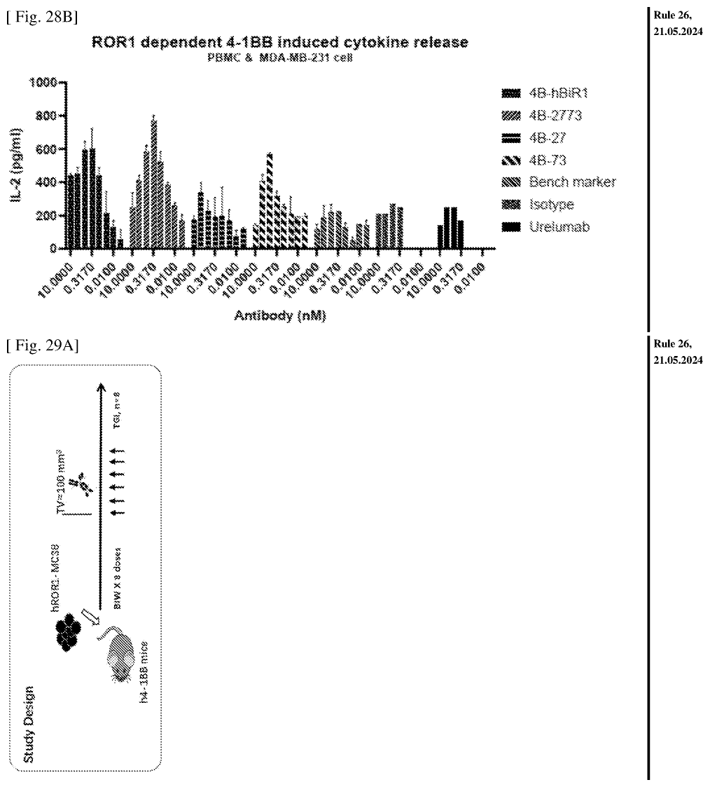

4B-2773, 4B-27 and 4B-73 are selected as a representative of engineered mono-topic and bi-paratopic ROR1×4-1BB antibodies to compare with 4B-hBiR1 and the bench marker (Clone ID: BA6(NA)×1A10M12) in a cytokine release assay. 1 μg/ml anti-CD3 (OKT3) were coated on plate, MDA-MB-231 cell line was used as target cell, and PBMC and gradient-diluted antibodies were co-cultured for 72 hours. IFN-γ and IL-2 induced by 4-1BB were detected. The results showed that all the test engineered ROR1×4-1BB antibodies released higher cytokines than the bench marker and the urelumab (FIGS. 28A-28B).

### In Vivo Efficacy

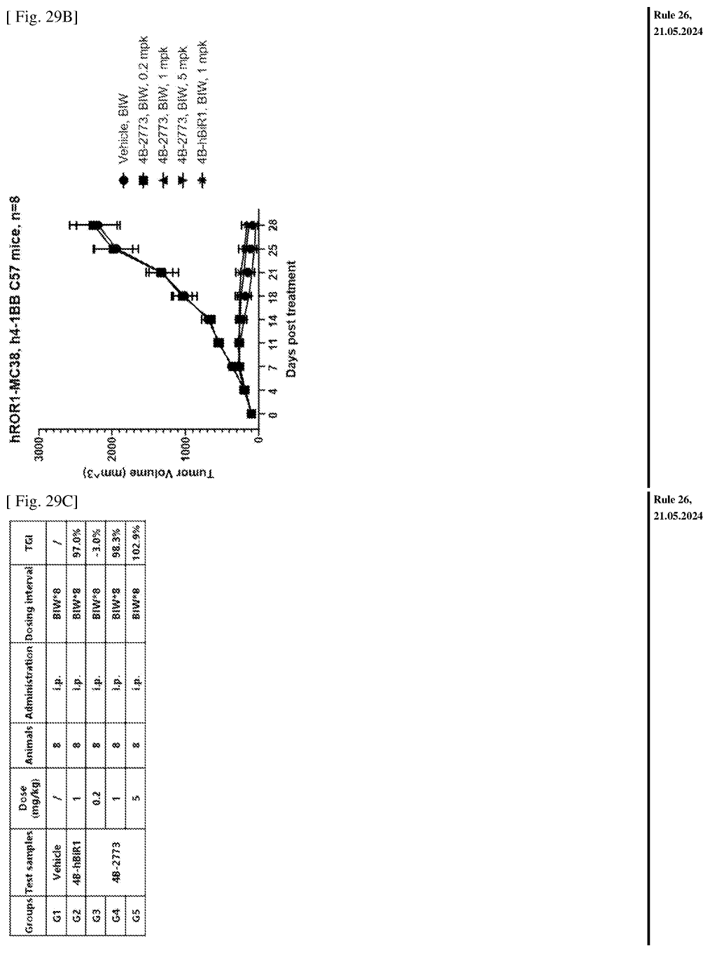

A syngeneic model (4-1BB humanized mice (Biocytogen)) was used to evaluate the in vivo efficacy of the engineered mono-topic and bi-paratopic ROR1×4-1BBs inoculated with the ROR1-MC38 cell line. All the tested humanized antibodies showed significant inhibition to the tumor growth (FIGS. 29A-29C).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Summary Table of Amino Acid Sequences A

| | | SEQ ID |
|---|---|---|
| DESCRIPTION | SEQUENCE | NO: |
| CLDN6-1 | SGYYWN | 1 |
| VH-CDR1 | | |
| CLDN6-1 | YISYSGSNNYNPSLKN | 2 |
| VH-CDR2 | | |
| CLDN6-1 | GGYYFDY | 3 |
| VH-CDR3 | | |
| CLDN6-1 | KSSQSLLNTNNQKNYLA | 4 |
| VL-CDR1 | | |
| CLDN6-1 | FASTRES | 5 |
| VL-CDR2 | | |
| CLDN6-1 | QQHYNAPRT | 6 |
| VL-CDR3 | | |
| CLDN6-1 | QVQLQESGPGLVKPSETLSLTCTVSGYSITSGYYWNWIRQPPGKGLEWMGYISYSG | 7 |
| VH | SNNYNPSLKNRVTISRDTSKNQFSLKLSSVTAADTAVYYCARGGYYFDYWGQGTLV | |
| | TVSS | |
| CLDN6-1 | DIVMTQSPDSLAVSLGERATINCKSSQSLLNTNNQKNYLAWYQQKPGQPPKLLIYF | 8 |
| VL | ASTRESGVPDRFSGSGSGTDFTLTISSLQAEDVAVYFCQQHYNAPRTFGGGTKVEI | |
| | K | |
| HC of | QVQLQESGPGLVKPSETLSLTCTVSGYSITSGYYWNWIRQPPGKGLEWMGYISYSG | 9 |
| CLDN6-1 WT | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGK | |
| HC of | QVQLQESGPGLVKPSETLSLTCTVSGYSITSGYYWNWIRQPPGKGLEWMGYISYSG | 10 |
| CLDN6-1 | SNNYNPSLKNRVTISRDTSKNQFSLKLSSVTAADTAVYYCARGGYYFDYWGQGTLV | |
| N297A | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYASTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGK | |
| LC of | DIVMTQSPDSLAVSLGERATINCKSSQSLLNTNNQKNYLAWYQQKPGQPPKLLIYF | 11 |
| CLDN6-1 | ASTRESGVPDRFSGSGSGTDFTLTISSLQAEDVAVYFCQQHYNAPRTFGGGTKVEI | |
| | KRTVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |
| CLDN6-3 | IYGMS | 12 |
| VH-CDR1 | | |
| CLDN6-3 | TISRDASYTYFPDSVKG | 13 |
| VH-CDR2 | | |
| CLDN6-3 | LGDNDRGYAMDY | 14 |
| VH-CDR3 | | |
| CLDN6-3 | RSSQSVVHVNANTYLE | 15 |
| VL-CDR1 | | |
| CLDN6-3 | KVSNRFP | 16 |
| VL-CDR2 | | |
| CLDN6-3 | FQGSHVPRT | 17 |
| VL-CDR3 | | |
| CLDN6-3 | EVQLVESGGGLVQPGGSLRLSCAASGFTFSIYGMSWVRQAPGKGLEWVATISRDAS | 18 |
| VH | YTYFPDSVKGRFTISRDNAKNSLYLQMNSLRAEDTAVYYCVRLGDNDRGYAMDYWG | |
| | QGTLVTVSS | |
| CLDN6-3 | DVVMTQSPLSLPVTLGQPASISCRSSQSVVHVNANTYLEWYQQRPGQSPRLLIYKV | 19 |
| VL | SNRFPGVPDRFSGSGSGTDFTLKISRVEAEDVGVYYCFQGSHVPRTFGGGTKVEIK | |
| HC of | EVQLVESGGGLVQPGGSLRLSCAASGFTFSIYGMSWVRQAPGKGLEWVATISRDAS | 20 |
| CLDN6-3 WT | YTYFPDSVKGRFTISRDNAKNSLYLQMNSLRAEDTAVYYCVRLGDNDRGYAMDYWG | |
| | QGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSC | |
| | DKTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGK | |
| HC of | EVQLVESGGGLVQPGGSLRLSCAASGFTFSIYGMSWVRQAPGKGLEWVATISRDAS | 21 |
| CLDN6-3 | YTYFPDSVKGRFTISRDNAKNSLYLQMNSLRAEDTAVYYCVRLGDNDRGYAMDYWG | |
| N297A | QGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSC | |
| | DKTHTCPPCPAPELLGGPSVFLFPPKPKDTLMISRTPEVTCVVVDVSHEDPEVKEN | |
| | WYVDGVEVHNAKTKPREEQYASTYRVVSVLTVLHQDWLNGKEYKCKVSNKALPAPI | |
| | EKTISKAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGK | |
| LC of | DVVMTQSPLSLPVTLGQPASISCRSSQSVVHVNANTYLEWYQQRPGQSPRLLIYKV | 22 |
| CLDN6-3 | SNRFPGVPDRFSGSGSGTDFTLKISRVEAEDVGVYYCFQGSHVPRTFGGGTKVEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |
| Linker | GGGGSGGGGSGGGGS | 23 |
| 4-1BB CDR1 | SNCMG | 24 |
| 4-1BB CDR2 | VICTGGGSPSYADSVKG | 25 |
| 4-1BB CDR3 | DLLRAGTPLSSYEFNY | 26 |
| 4-1BB | QVQLQESGGGLVQPGGSLRLSCAASGYTYSSNCMGWFRQAPGKGLEGVAVICTGGG | 27 |
| VHH | SPSYADSVKGRFTISRDNAKNTLYLQMNSLRAEDTAVYYCAADLLRAGTPLSSYEF | |
| | NYWGQGTLVTVSS | |
| Heavy chain | QVQLQESGPGLVKPSETLSLTCTVSGYSITSGYYWNWIRQPPGKGLEWMGYISYSG | 28 |
| component | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYASTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGKGG | |
| | GGSGGGGSGGGGSQVQLQESGGGLVQPGGSLRLSCAASGYTYSSNCMGWFRQAPGK | |
| | GLEGVAVICTGGGSPSYADSVKGRFTISRDNAKNTLYLQMNSLRAEDTAVYYCAAD | |
| | LLRAGTPLSSYEFNYWGQGTLVTVSS | |
| Light chain | DIVMTQSPDSLAVSLGERATINCKSSQSLLNTNNQKNYLAWYQQKPGQPPKLLIYF | 29 |
| component | ASTRESGVPDRFSGSGSGTDFTLTISSLQAEDVAVYFCQQHYNAPRTFGGGTKVEI | |
| | KRTVAAPSVFIFPPSDEQLKSGTASVVCLLNNFYPREAKVOWKVDNALQSGNSQES | |
| | VTEQDSKDSTYSLSSTLTLSKADYEKHKVYACEVTHQGLSSPVTKSENRGEC | |
| Heavy chain | QVQLQESGPGLVKPSETLSLTCTVSGYSITSGYYWNWIRQPPGKGLEWMGYISYSG | 30 |
| component | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGKGG | |
| | GGSGGGGSGGGGSQVQLQESGGGLVQPGGSLRLSCAASGYTYSSNCMGWFRQAPGK | |
| | GLEGVAVICTGGGSPSYADSVKGRFTISRDNAKNTLYLQMNSLRAEDTAVYYCAAD | |
| | LLRAGTPLSSYEFNYWGQGTLVTVSS | |
| Light chain | DIVMTQSPDSLAVSLGERATINCKSSQSLLNTNNQKNYLAWYQQKPGQPPKLLIYF | 31 |
| component | ASTRESGVPDRFSGSGSGTDFTLTISSLQAEDVAVYFCQQHYNAPRTFGGGTKVEI | |
| | KRTVAAPSVFIFPPSDEQLKSGTASVVCLLNNFYPREAKVQWKVDNALQSGNSQES | |
| | VTEQDSKDSTYSLSSTLTLSKADYEKHKVYACEVTHQGLSSPVTKSENRGEC | |
| Heavy chain | QVQLQESGGGLVQPGGSLRLSCAASGYTYSSNCMGWFRQAPGKGLEGVAVICTGGG | 32 |
| component | SPSYADSVKGRFTISRDNAKNTLYLQMNSLRAEDTAVYYCAADLLRAGTPLSSYEF | |
| | NYWGQGTLVTVSSGGGGSGGGGSGGGGSQVQLQESGPGLVKPSETLSLTCTVSGYS | |
| | ITSGYYWNWIRQPPGKGLEWMGYISYSGSNNYNPSLKNRVTISRDTSKNQFSLKLS | |
| | SVTAADTAVYYCARGGYYFDYWGQGTLVTVSSASTKGPSVFPLAPSSKSTSGGTAA | |
| | LGCLVKDYFPEPVTVSWNSGALTSGVHTFPAVLQSSGLYSLSSVVTVPSSSLGTQT | |
| | YICNVNHKPSNTKVDKKVEPKSCDKTHTCPPCPAPELLGGPSVFLFPPKPKDTLMI | |
| | SRTPEVTCVVVDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSREEMTKNQVSL | |
| | TCLVKGFYPSDIAVEWESNGQPENNYKTTPPVLDSDGSFFLYSKLTVDKSRWQQGN | |
| | VFSCSVMHEALHNHYTQKSLSLSPGK | |
| Light chain | DIVMTQSPDSLAVSLGERATINCKSSQSLLNTNNQKNYLAWYQQKPGQPPKLLIYF | 33 |
| component | ASTRESGVPDRFSGSGSGTDFTLTISSLQAEDVAVYFCQQHYNAPRTFGGGTKVEI | |
| | KRTVAAPSVFIFPPSDEQLKSGTASVVCLLNNFYPREAKVOWKVDNALQSGNSQES | |
| | VTEQDSKDSTYSLSSTLTLSKADYEKHKVYACEVTHQGLSSPVTKSENRGEC | |
| Heavy chain | QVQLQESGPGLVKPSETLSLTCTVSGYSITSGYYWNWIRQPPGKGLEWMGYISYSG | 34 |
| component | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGK | |
| Light chain | QVQLQESGGGLVQPGGSLRLSCAASGYTYSSNCMGWFRQAPGKGLEGVAVICTGGG | 35 |
| component | SPSYADSVKGRFTISRDNAKNTLYLQMNSLRAEDTAVYYCAADLLRAGTPLSSYEF | |
| | NYWGQGTLVTVSSGGGGSGGGGSGGGGSDIVMTQSPDSLAVSLGERATINCKSSQS | |
| | LLNTNNQKNYLAWYQQKPGQPPKLLIYFASTRESGVPDRFSGSGSGTDFTLTISSL | |
| | QAEDVAVYFCQQHYNAPRTFGGGTKVEIKRTVAAPSVFIFPPSDEQLKSGTASVVC | |
| | LLNNFYPREAKVQWKVDNALQSGNSQESVTEQDSKDSTYSLSSTLTLSKADYEKHK | |
| | VYACEVTHQGLSSPVTKSFNRGEC | |
| Heavy chain | EVQLVESGGGLVQPGGSLRLSCAASGFTFSIYGMSWVRQAPGKGLEWVATISRDAS | 36 |
| component | YTYFPDSVKGRFTISRDNAKNSLYLQMNSLRAEDTAVYYCVRLGDNDRGYAMDYWG | |
| | QGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSC | |
| | DKTHTCPPCPAPELLGGPSVFLFPPKPKDTLMISRTPEVTCVVVDVSHEDPEVKEN | |
| | WYVDGVEVHNAKTKPREEQYASTYRVVSVLTVLHQDWLNGKEYKCKVSNKALPAPI | |
| | EKTISKAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGKGGGGSGGGGSGGGGSQVQLQESGGGLVQPGGSLRLSCAASGYTYSSNCMGWFR | |
| | QAPGKGLEGVAVICTGGGSPSYADSVKGRFTISRDNAKNTLYLQMNSLRAEDTAVY | |
| | YCAADLLRAGTPLSSYEFNYWGQGTLVTVSS | |
| Light chain | DVVMTQSPLSLPVTLGQPASISCRSSQSVVHVNANTYLEWYQQRPGQSPRLLIYKV | 37 |
| component | SNRFPGVPDRFSGSGSGTDFTLKISRVEAEDVGVYYCFQGSHVPRTFGGGTKVEIK | |
| | RTVAAPSVFIFPPSDEQLKSGTASVVCLLNNFYPREAKVOWKVDNALQSGNSQESV | |
| | TEQDSKDSTYSLSSTLTLSKADYEKHKVYACEVTHQGLSSPVTKSFNRGEC | |
| Heavy chain | EVQLVESGGGLVQPGGSLRLSCAASGFTFSIYGMSWVROAPGKGLEWVATISRDAS | 38 |
| component | YTYFPDSVKGRFTISRDNAKNSLYLQMNSLRAEDTAVYYCVRLGDNDRGYAMDYWG | |
| | QGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSC | |
| | DKTHTCPPCPAPELLGGPSVFLFPPKPKDTLMISRTPEVTCVVVDVSHEDPEVKEN | |
| | WYVDGVEVHNAKTKPREEQYNSTYRVVSVLTVLHQDWLNGKEYKCKVSNKALPAPI | |
| | EKTISKAKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGKGGGGSGGGGSGGGGSQVQLQESGGGLVQPGGSLRLSCAASGYTYSSNCMGWFR | |
| | QAPGKGLEGVAVICTGGGSPSYADSVKGRFTISRDNAKNTLYLQMNSLRAEDTAVY | |
| | YCAADLLRAGTPLSSYEFNYWGQGTLVTVSS | |
| Light chain | DVVMTQSPLSLPVTLGQPASISCRSSQSVVHVNANTYLEWYQQRPGQSPRLLIYKV | 39 |
| component | SNRFPGVPDRFSGSGSGTDFTLKISRVEAEDVGVYYCFQGSHVPRTFGGGTKVEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |
| CLDN6 | MASAGMQILGVVLTLLGWVNGLVSCALPMWKVTAFIGNSIVVAQVVWEGLWMSCVV | 40 |
| | QSTGQMOCKVYDSLLALPQDLQAARALCVIALLVALFGLLVYLAGAKCTTCVEEKD | |
| | SKARLVLTSGIVFVISGVLTLIPVCWTAHAIIRDFYNPLVAEAQKRELGASLYLGW | |
| | AASGLLLLGGGLLCCTCPSGGSQGPSHYMARYSTSAPAISRGPSEYPTKNYV | |
| 4-1BB | LQDPCSNCPAGTFCDNNRNQICSPCPPNSESSAGGQRTCDICROCKGVERTRKECS | 41 |
| | STSNAECDCTPGFHCLGAGCSMCEQDCKQGQELTKKGCKDCCFGTENDQKRGICRP | |
| | WTNCSLDGKSVLVNGTKERDVVCGPSPADLSPGASSVTPPAPAREPGHSPQIISFF | |
| | LALTSTALLFLLFFLTLRFSVVKRGRKKLLYIFKOPFMRPVQTTQEEDGCSCRFPE | |
| | EEEGGCEL | |
| HC of | QVQLQESGPGLVKPSETLSLTCTVSGYSITSGYYWNWIRQPPGKGLEWMGYISYSG | 42 |
| CLDN6-1 WT | SNNYNPSLKNRVTISRDTSKNOFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPG | |
| HC of | QVQLQESGPGLVKPSETLSLTCTVSGYSITSGYYWNWIRQPPGKGLEWMGYISYSG | 43 |
| CLDN6-1 | SNNYNPSLKNRVTISRDTSKNOFSLKLSSVTAADTAVYYCARGGYYFDYWGQGTLV | |
| N297A | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYASTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPG | |
| Heavy chain | QVQLQESGPGLVKPSETLSLTCTVSGYSITSGYYWNWIRQPPGKGLEWMGYISYSG | 44 |
| component | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYASTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGGGG | |
| | GSGGGGSGGGGSQVQLQESGGGLVQPGGSLRLSCAASGYTYSSNCMGWFRQAPGKG | |
| | LEGVAVICTGGGSPSYADSVKGRFTISRDNAKNTLYLQMNSLRAEDTAVYYCAADL | |
| | LRAGTPLSSYEFNYWGQGTLVTVSS | |
| Heavy chain | QVQLQESGPGLVKPSETLSLTCTVSGYSITSGYYWNWIRQPPGKGLEWMGYISYSG | 45 |
| component | SNNYNPSLKNRVTISRDTSKNQFSLKLSSVTAADTAVYYCARGGYYFDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKENWYVDG | |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGGGG | |
| | GSGGGGSGGGGSQVQLQESGGGLVQPGGSLRLSCAASGYTYSSNCMGWFRQAPGKG | |
| | LEGVAVICTGGGSPSYADSVKGRFTISRDNAKNTLYLQMNSLRAEDTAVYYCAADL | |
| | LRAGTPLSSYEFNYWGQGTLVTVSS | |
| IgG1 Fc | CPPCPAPELLGGPSVFLFPPKPKDTLMISRTPEVTCVVVDVSHEDPEVKENWYVDG | 46 |
| domain (WT) | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 N297A | CPPCPAPELLGGPSVFLFPPKPKDTLMISRTPEVTCVVVDVSHEDPEVKENWYVDG | 47 |
| | VEVHNAKTKPREEQYASTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 N297Q | CPPCPAPELLGGPSVFLFPPKPKDTLMISRTPEVTCVVVDVSHEDPEVKENWYVDG | 48 |
| | VEVHNAKTKPREEQYQSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTOKSLSLSP | |
| IgG1 N297G | CPPCPAPELLGGPSVFLFPPKPKDTLMISRTPEVTCVVVDVSHEDPEVKENWYVDG | 49 |
| | VEVHNAKTKPREEQYGSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTOKSLSLSP | |
| IgG1 LALA | CPPCPAPEAAGGPSVFLFPPKPKDTLMISRTPEVTCVVVDVSHEDPEVKENWYVDG | 50 |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 LALA- | CPPCPAPEAAGGPSVFLFPPKPKDTLMISRTPEVTCVVVDVSHEDPEVKENWYVDG | 51 |
| PG | VEVHNAKTKPREEQYNSTYRVVSVLTVLHQDWLNGKEYKCKVSNKALGAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 FES | CPPCPAPEFEGGPSVFLFPPKPKDTLMISRTPEVTCVVVDVSHEDPEVKENWYVDG | 52 |
| | VEVHNAKTKPREEQYNSTYRVVSVLTVLHQDWLNGKEYKCKVSNKALPASIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTOKSLSLSP | |
| IgG1 FEA | CPPCPAPEFEGGPSVFLFPPKPKDTLMISRTPEVTCVVVAVSHEDPEVKENWYVDG | 53 |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 PD | CPPCPAPELLGGDSVFLFPPKPKDTLMISRTPEVTCVVVDVSHEDPEVKENWYVDG | 54 |
| | VEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 SELF | CPPCPAPELLGGPSVFLFPPKPKDTLMISRTPEVTCVVVDVEHEDPEVKENWYVDG | 55 |
| | VEVHNAKTKPREEQYNSTYRVVSVLTVLHQDWLNGKEYKCKVSNKAFPAPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 V12 | CPPCPAPDLLGDDSVFLFPPKPKDTLMISRTPEVTCVVVDVSDEDGEVKFNWYVDG | 56 |
| | VEVHNAKTKPREEQYNSTYRVVSVLTVLHQDWLNGKEYKCKVSNKALPRPIEKTIS | |
| | KAKGQPREPQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSP | |
| IgG1 (S237C | CPPCPAPELLGGPSVFLFPPKPKDTLMISRTPEVTCVVVDVSHEDPEVKFNWYVDG | 285 |
| and T249W) | VEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKT | |
| | TPPVLDSDGSFFLYSKLTVDKSRWQQGNVFSCSVMHEALHNHYTQKSLSLSPGK | |
| IgG1 (S237C, | CPPCPAPELLGGPSVFLFPPKPKDTLMISRTPEVTCVVVDVSHEDPEVKFNWYVDG | 286 |
| T249W, | VEVHNAKTKPREEQYNSTYRVVSVLTVLHQDWLNGKEYKCKVSNKALPAPIEKTIS | |
| M311L and | KAKGQPREPQVYTLPPCREEMTKNQVSLWCLVKGFYPSDIAVEWESNGQPENNYKT | |
| N317S) | TPPVLDSDGSFFLYSKLTVDKSRWQQGNVFSCSVLHEALHSHYTQKSLSLSPGK | |
| IgG (Y136C, | CPPCPAPELLGGPSVFLFPPKPKDTLMISRTPEVTCVVVDVSHEDPEVKENWYVDG | 288 |
| T153S, L155A | VEVHNAKTKPREEQYNSTYRVVSVLTVLHQDWLNGKEYKCKVSNKALPAPIEKTIS | |
| and Y194V) | KAKGQPREPQVCVTLPPSREEMTKNQVSLSVCAVKGFYPSDIAVEWESNGQPENNY | |
| | KTTPPVLDSDGSFFLVVSKLTVDKSRWQQGNVFSCSVMHEALHNHYTQKSLSLSPG | |
| | K | |
| IgG (Y136C, | CPPCPAPELLGGPSVFLFPPKPKDTLMISRTPEVTCVVVDVSHEDPEVKFNWYVDG | 289 |
| T153S, | VEVHNAKTKPREEQYNSTYRVVSVLTVLHQDWLNGKEYKCKVSNKALPAPIEKTIS | |
| L155A, | KAKGQPREPQVCTLPPSREEMTKNQVSLSCAVKGFYPSDIAVEWESNGQPENNYKT | |
| Y194V, | TPPVLDSDGSFFLVSKLTVDKSRWQQGNVFSCSVLHEALHSHYTQKSLSLSPGK | |
| M215L and | | |
| N221S) | | |
| M16-1 Heavy | QVQLVESGGGLVKPGGSLRLSCAASGFTFSNYYMSWVRQAPGKGLEWISYISGRGS | 182 |
| chain | TIFYADSVKGRITISRDNAKNSLFLQMNSLRAEDTAVYFCVKDRGGYSPYWGQGTL | |
| | VTVSSASTKGPSVFPLAPSSKSTSGGTAALGCLVKDYFPEPVTVSWNSGALTSGVH | |
| | TFPAVLQSSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEVKFNWYVD | |
| | GVEVHNAKTKPREEQYASTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| M16-1 Light | DIQMTQSPSSLSASVGDRVTITCRASQSISTYLNWYQQKPGKAPKLLIYTASSLQS | 183 |
| chain | GVPSRFSGSGSGTDFTLTISSLQPEDFATYYCQQSYSTPPITFGQGTRLEIKRTVA | |
| | APSVFIFPPSDEQLKSGTASVVCLLNNFYPREAKVQWKVDNALQSGNSQESVTEQD | |
| | SKDSTYSLSSTLTLSKADYEKHKVYACEVTHQGLSSPVTKSENRGEC | |
| M16-2 Heavy | EVQLVESGGGLEQPGRSLRLSCTASGFAFGDHTMSWVRQAPGKGLEWVGFIRSRAY | 184 |
| chain | GGTTEYAASVKGRFTISRDDSKSIAYLQMDSLKTEDTAVYYCTSGGYDSSLHYYYY | |
| | YHGMDVWGRGTTVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVDVSH | |
| | EDPEVKFNWYVDGVEVHNAKTKPREEQYASTYRVVSVLTVLHQDWLNGKEYKCKVS | |
| | NKALPAPIEKTISKAKGQPREPQVYTLPPSREEMTKNQVSLTCLVKGFYPSDIAVE | |
| | WESNGQPENNYKTTPPVLDSDGSFFLYSKLTVDKSRWQQGNVFSCSVMHEALHNHY | |
| | TQKSLSLSPGK | |
| M16-2 Light | EIVLTQSPGTLSLSPGERATLSCRASQSVSSSYLAWYQQKPGQAPRLLIYGASSRA | 185 |
| chain | TGIPDRFSGSGSGTDFTLTISRLEPEDFAVYYCQQYGSSPWTFGQGTKVEIKRTVA | |
| | APSVFIFPPSDEQLKSGTASVVCLLNNFYPREAKVQWKVDNALQSGNSQESVTEQD | |
| | SKDSTYSLSSTLTLSKADYEKHKVYACEVTHQGLSSPVTKSFNRGEC | |
| 3A5 Heavy | EVQLVESGGGLVQPGGSLRLSCAASGYSITNDYAWNWVRQAPGKGLEWVGYISYSG | 186 |
| chain | YTTYNPSIKSRFTISRDTSKNTLYLQMNSLRAEDTAVYYCARWTSGLDYWGQGTLV | |
| | TVSSASTKGPSVFPLAPSSKSTSGGTAALGCLVKDYFPEPVTVSWNSGALTSGVHT | |
| | FPAVLQSSGLYSLSSVVTVPSSSLGTQTYICNVNHKPSNTKVDKKVEPKSCDKTHT | |
| | CPPCPAPELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPAPIEKTIS | |
| | KAKGQPREPOVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSLSLSPGK | |
| 3A5 | DIQMTQSPSSLSASVGDRVTITCKASDLIHNWLAWYQQKPGKAPKLLIYGATSLET | 187 |
| Light chain | GVPSRFSGSGSGTDFTLTISSLQPEDFATYYCQQYWTTPFTFGQGTKVEIKRTVAA | |
| | PSVFIFPPSDEQLKSGTASVVCLLNNFYPREAKVQWKVDNALQSGNSQESVTEQDS | |
| | KDSTYSLSSTLTLSKADYEKHKVYACEVTHQGLSSPVTKSFNRGEC | |

Summary Table of Amino Acid Sequences B (anti-RORI)

| Antibody | | | SEQ ID |
|---|---|---|---|
| chain | Sequences | | NO: |
| 3C5 | VH | EVKLVESGGGLVKPGGSLKLSCAASGFTFSSYAMSWVRQTPEKRLEWVSSISSGGTT | 188 |
| | | YYPDTVKGRFTISRDNARNILYLQMSSLRSEDTAMYYCARDSLYYGSSLYYAMDYWG | |
| | | QGTSVTVSS | |
| | VL | DIKMTQSPSSMYASLGERVSITCRASQDIYSYLSWFQQKPGKSPKTLIYRANRLVDG | 189 |
| | | VPSRFSGSGSGQDYSLTISSLEYEDMGIYYCLQYDEFPYTFGGGTKLEIK | |
| | HCDR1 | SYAMS | 190 |
| | HCDR2 | SISSGGTTYYPDTVKGR | 191 |
| | HCDR3 | DSLYYGSSLYYAMDY | 192 |
| | LCDR1 | RASQDIYSYLS | 193 |
| | LCDR2 | RANRLVDG | 194 |
| | LCDR3 | LQYDEFPYT | 195 |
| 8F2 | VH | EVQLQQSGPELVKPGASVKMSCKASGYTFTTYVMHWVKQKPGQGLEWIGYINPYNGG | 196 |
| | | IRYNEKFKGKATLTSDKSSSTAYMELSSLTSEDSAVYYCARRERGVYYGMDEWGQGT | |
| | | SVTVSS | |
| | VL | DVVMTQTPLTLSVTIGQPASISCKSSQSLLHSNGKTYLNWLLQRPGQSPNLLIYLVS | 197 |
| | | KLESGVPDRFSGSGSGTDFTLKISRVEAEDLGVYYCLQATYFPYTFGSGTKLEIK | |
| | HCDR1 | TYVMH | 198 |
| | HCDR2 | YINPYNGGIRYNEKFKG | 199 |
| | HCDR3 | RERGVYYGMDE | 200 |
| | LCDR1 | KSSQSLLHSNGKTYLN | 201 |
| | LCDR2 | LVSKLESG | 202 |
| | LCDR3 | LQATYFPYT | 203 |
| 9C9 | VH | EVKLVESGPVLVAPSQSLSITCTVSGFSLTSYGVHWVRQPPGKGLEWLGVIWAGGHT | 204 |
| | | NYNSDLMSRLSISKDNSKSQVFLKMNSLQTDDTAMYYCVKRSIYGDYALDYWGQGTS | |
| | | VTVSS | |
| | VL | DIVLTQSPAITAASLGQKVTITCSASSSVTYMHWYQQKSGTSPKLWIYDISKLASGV | 205 |
| | | PARFSGSGPGTSYSLTISTMEAEDAAIYYCQQWNYPLMTFGGGTKLEIK | |
| | HCDR1 | SYGVH | 206 |
| | HCDR2 | VIWAGGHTNYNSDLMSR | 207 |
| | HCDR3 | RSIYGDYALDY | 208 |
| | LCDR1 | VTYMH | 209 |
| | LCDR2 | DISKLASG | 210 |
| | LCDR3 | QQWNYPLMT | 211 |
| 10C9 | VH | QVQLQQPGAELVMPGASVKMSCKASGYKFTDYWMHWVKQRPGQGLEWIGAIDTSDSS | 212 |
| | | TRNNQKFKGKATLTVDESSSTAYMQLSRLTSEDSAVYYCARGARTGTGFGYWGQGTL | |
| | | VTVSS | |
| | VL | DVVMTQTPLTLSVAIGQPASISCKSSQSLLHINGKTYLNWLLQRPGQSPKLLIYLVS | 213 |
| | | KLESGVPDRFSGSGSGTDFTLKISRVEAEDLGVYYCLQATHEPYTFGSGTKLEIK | |
| | HCDR1 | DYWMH | 214 |
| | HCDR2 | AIDTSDSSTRNNQKFKG | 215 |
| | HCDR3 | GARTGTGFGY | 216 |
| | LCDR1 | KSSQSLLHINGKTYLN | 217 |
| | LCDR2 | LVSKLESG | 202 |
| | LCDR3 | LQATHFPYT | 219 |
| h3C5- | VH.SA | EVQLVESGGGLVKPGGSLRLSCAASGFTFSSYAMSWVRQAPGKRLEWVSSISSGGTT | 222 |
| PTM | | YYPDTVKGRFTISRDNAKNSLYLQMNSLRAEDTAVYYCARDALYYGSSLYYAMDYWG | |
| | | QGTLVTVSS | |
| | VL.GA | DIQMTQSPSSLSASVGDRVTITCRASQDIYSYLSWFQQKPGKAPKTLIYRANRLVDA | 223 |
| | | VPSRFSGSGSGQDYTLTISSLQPEDFATYYCLQYDEFPYTFGQGTKLEIK | |
| | HCDR1 | SYAMS | 190 |
| | HCDR2 | SISSGGTTYYPDTVKGR | 191 |
| | HCDR3 | DALYYGSSLYYAMDY | 220 |
| | LCDR1 | RASQDIYSYLS | 193 |
| | LCDR2 | RANRLVDA | 221 |
| | LCDR3 | LQYDEFPYT | 195 |
| h8F2 | VH | QVQLVQSGAEVKKPGASVKVSCKASGYTFTTYVMHWVRQAPGQGLEWMGYINPYNGG | 224 |
| | | IRYNEKFKGRVTMTRDTSTSTVYMELSSLRSEDTAVYYCARRERGVYYGMDEWGQGT | |
| | | LVTVSS | |
| | VL | DIVMTQTPLSLSVTPGQPASISCKSSQSLLHSNGKTYLNWYLQKPGQSPQLLIYLVS | 225 |
| | | KLESGVPDRFSGSGSGTDFTLKISRVEAEDVGVYYCLQATYFPYTFGQGTKLEIK | |
| | HCDR | TYVMH | 198 |
| | HCDR2 | YINPYNGGIRYNEKFKG | 199 |
| | HCDR3 | RERGVYYGMDE | 200 |
| | LCDR1 | KSSQSLLHSNGKTYLN | 201 |
| | LCDR2 | LVSKLESG | 202 |
| | LCDR3 | LQATYFPYT | 203 |
| 3C5-27 | VH | EVQLVESGGGLVKPGGSLRLSCAASGFTFSSYAMSWVRQAPGKRLEWVSSISSGGTT | 226 |
| | | YYPDTVKGRFTISRDNAKNSLYLQMNSLRAEDTAVYYCARDALYYGGSLYYAMDYWG | |
| | | QGTLVTVSS | |
| | VL | DIQMTQSPSSLSASVGDRVTITCRASQDIYSYLSWFQQKPGKAPKTLIYRANRLVDA | 223 |
| | | VPSRFSGSGSGODYTLTISSLQPEDFATYYCLQYDEFPYTFGQGTKLEIK | |
| | HCDR1 | SYAMS | 190 |
| | HCDR2 | SISSGGTTYYPDTVKGR | 191 |
| | HCDR3 | DALYYGGSLYYAMDY | 227 |
| | LCDR1 | RASQDIYSYLS | 193 |
| | LCDR2 | RANRLVDA | 221 |
| | LCDR3 | LQYDEFPYT | 195 |
| 3C5-29 | VH | EVQLVESGGGLVKPGGSLRLSCAASGFTFSRYAMSWVRQAPGKRLEWVSSISSGGNT | 228 |
| | | YYPDTVKGRFTISRDNAKNSLYLQMNSLRAEDTAVYYCARDALYYGSSLYYAMDYWG | |
| | | QGTLVTVSS | |
| | VL | DIQMTQSPSSLSASVGDRVTITCRASQDIYSYLSWFQQKPGKAPKTLIYRENRLVDA | 229 |
| | | VPSRFSGSGSGQDYTLTISSLQPEDFATYYCLQYDEFPYTFGQGTKLEIK | |
| | HCDR1 | GFTFSRYAMS | 230 |
| | HCDR2 | SISSGGNTYYPDTVKGR | 231 |
| | HCDR3 | DALYYGSSLYYAMDY | 220 |
| | LCDR1 | RASQDIYSYLS | 193 |
| | LCDR2 | RENRLVDA | 232 |
| | LCDR3 | LQYDEFPYT | 195 |
| 3C5-32 | VH | EVQLVESGGGLVKPGGSLRLSCAASGFTFRSYAMSWVRQAPGKRLEWVSSISSGGTR | 234 |
| | | YYPDTVKGRFTISRDNAKNSLYLQMNSLRAEDTAVYYCARDALYYGSSLYYAMDYWG | |
| | | QGTLVTVSS | |
| | VL | DIQMTQSPSSLSASVGDRVTITCRASQDIYSYLSWFQQKPGKAPKTLIYRENRLVDA | 229 |
| | | VPSRFSGSGSGQDYTLTISSLQPEDFATYYCLQYDEFPYTFGQGTKLEIK | |
| | HCDR1 | SYAMS | 190 |
| | HCDR2 | SISSGGTRYYPDTVKGR | 233 |
| | HCDR3 | DALYYGSSLYYAMDY | 220 |
| | LCDR1 | RASQDIYSYLS | 193 |
| | LCDR2 | RENRLVDA | 232 |
| | LCDR3 | LQYDEFPYT | 195 |
| 8F2-73 | VH | QVQLVQSGAEVKKPGASVKVSCKASGYRFTTYVMHWVRQAPGQGLEWMGYINPYNGV | 235 |
| | | IRYNEKFKGRVTMTRDTSTSTVYMELSSLRSEDTAVYYCARRERGVYYGMDEWGQGT | |
| | | LVTVSS | |
| | VL | DIVMTQTPLSLSVTPGQPASISCKSSQSLLHSNGKTYLNWYLQKPGQSPQLLIYLVS | 236 |
| | | KLSSGVPDRFSGSGSGTDFTLKISRVEAEDVGVYYCLQATYFPYTFGQGTKLEIK | |
| | HCDR1 | TYVMH | 198 |
| | HCDR2 | YINPYNGVIRYNEKFKG | 238 |
| | HCDR3 | RERGVYYGMDE | 200 |
| | LCDR1 | KSSQSLLHSNGKTYLN | 201 |
| | LCDR2 | LVSKLSSG | 239 |
| | LCDR3 | LQATYFPYT | 203 |
| 8F2-87 | VH | QVQLVQSGAEVKKPGASVKVSCKASGYTFTTYVMHWVRQAPGQGLEWMGYINPYNGG | 240 |
| | | IRYNEKFKGRVTMTRDTSTSTVYMELSSLRSEDTAVYYCARRERGVYYGMSEWGQGT | |
| | | LVTVSS | |
| | VL | DIVMTQTPLSLSVTPGQPASISCKSSQSLLHSNGKTYLNWYLQKPGQSPQLLIYLVS | 241 |
| | | KLESGVPDRFSGSGSGTDFTLKISRVEAEDVGVYYCYQATYFPYTFGQGTKLEIK | |
| | HCDR1 | TYVMH | 198 |
| | HCDR2 | YINPYNGGIRYNEKFKG | 199 |
| | HCDR3 | RERGVYYGMSE | 242 |
| | LCDR1 | KSSQSLLHSNGKTYLN | 201 |
| | LCDR2 | LVSKLESG | 202 |
| | LCDR3 | YQATYFPYT | 243 |
| 8F2-87DS | VH | QVQLVQSGAEVKKPGASVKVSCKASGYTFTTYVMHWVRQAPGQGLEWMGYINPYSGG | 244 |
| (removal | | IRYNEKFKGRVTMTRDTSTSTVYMELSSLRSEDTAVYYCARRERGVYYGMSEWGQGT | |
| of | | LVTVSS | |
| potential | | | |
| deamidation | | | |
| site) | | | |
| | VL | DIVMTQTPLSLSVTPGQPASISCKSSQSLLHSNDKTYLNWYLQKPGQSPQLLIYLVS | 245 |
| | | KLESGVPDRFSGSGSGTDFTLKISRVEAEDVGVYYCYQATYFPYTFGQGTKLEIK | |
| | HCDR1 | TYVMH | 198 |
| | HCDR2 | YINPYSGGIRYNEKFKG | 246 |
| | HCDR3 | RERGVYYGMSE | 242 |
| | LCDR1 | KSSQSLLHSNDKTYLN | 247 |
| | LCDR2 | LVSKLESG | 202 |
| | LCDR3 | YQATYFPYT | 243 |
| 8F2-82 | VH | QVQLVQSGAEVKKPGASVKVSCKASGYTFTTYVMHWVRQAPGQGLEWMGYINPYSGG | 248 |
| | | IRYNEKFKGRVTMTRDTSTSTVYMELSSLRSEDTAVYYCARRERGVTAGMDEWGQGT | |
| | | LVTVSS | |
| | VL | DIVMTQTPLSLSVTPGQPASISCKSSQSLLHSNGKTYLNWYLQKPGQSPQLLIYLVS | 225 |
| | | KLESGVPDRFSGSGSGTDFTLKISRVEAEDVGVYYCLQATYFPYTFGQGTKLEIK | |
| | HCDR1 | TYVMH | 198 |
| | HCDR2 | YINPYSGGIRYNEKFKG | 246 |
| | HCDR3 | RERGVTAGMDE | 249 |
| | LCDR1 | KSSQSLLHSNGKTYLN | 201 |
| | LCDR2 | LVSKLESG | 202 |
| | LCDR3 | LQATYFPYT | 203 |
| 8F2-97 | VH | QVQLVQSGAEVKKPGASVKVSCKASGYTFTNYVMHWVRQAPGQGLEWMGYINPYSGG | 250 |
| | | IRYNEKFKGRVTMTRDTSTSTVYMELSSLRSEDTAVYYCARRERGVYYGMDEWGQGT | |
| | | LVTVSS | |
| | VL | DIVMTQTPLSLSVTPGQPASISCKSSQSLLHSNGKTYLNWYLQKPGQSPQLLIYLVS | 225 |
| | | KLESGVPDRFSGSGSGTDFTLKISRVEAEDVGVYYCLQATYFPYTFGQGTKLEIK | |
| | HCDR1 | NYVMH | 251 |
| | HCDR2 | YINPYSGGIRYNEKFKG | 246 |
| | HCDR3 | RERGVYYGMDE | 200 |
| | LCDR1 | KSSQSLLHSNGKTYLN | 201 |
| | LCDR2 | LVSKLESG | 202 |
| | LCDR3 | LQATYFPYT | 203 |

1. A multispecific construct comprising:
(1) a first antibody moiety that specifically binds to a tumor antigen; and
(2) a second antibody moiety that specifically binds to 4-1BB, wherein the binding of the first antibody moiety with the tumor antigen triggers the second antibody moiety to activate 4-1BB.

2. The multispecific construct of claim 1, further comprising a Fc domain with maintained or improved effector function.

3. (canceled)

4. The multispecific construct of claim 1, wherein the second antibody moiety specifically binds to CRD 3/4 region of 4-1BB.

5-6. (canceled)

7. The multispecific construct of claim 4, wherein the second antibody moiety is a sdAb which comprises a sdAb-CDR1, a sdAb-CDR2, and a sdAb-CDR3, respectively comprising the amino acid sequence of a CDR1, a CDR2, and a CDR3 within a single monomeric variable antibody domain comprising the amino acid sequence set forth in SEQ ID NO: 27.

8. (canceled)

9. The multispecific construct of claim 7, wherein the sdAb comprises:
(1) a sdAb-CDR1 comprising an amino acid sequence of SEQ ID NO: 24;
(2) a sdAb-CDR2 comprising an amino acid sequence of SEQ ID NO: 25; and
(3) a sdAb-CDR3 comprising an amino acid sequence of SEQ ID NO: 26.

10. The multispecific construct of claim 1, wherein the second antibody moiety comprises the amino acid sequence of SEQ ID NO:27, or a variant thereof having at least about 80% sequence identity to SEQ ID NO: 27.

11-13. (canceled)

14. The multispecific construct of claim 1, wherein the Fc domain comprises an amino acid sequence having at least 80% identity with any one of SEQ ID NOs: 46-56, 285-286 and 288-289.

15. The multispecific construct of claim 1, wherein the activation of 4-1BB by the second antibody moiety is enhanced by at least 10 folds after binding of the first antibody moiety with the tumor antigen.

16. The multispecific construct of claim 1, wherein the activation of the 4-1BB by the second antibody moiety results in an increase in IFN7 level, IL-2 level, or NFκB signaling.

17-22. (canceled)

23. The multispecific construct of claim 1, further comprises a third antibody moiety that specifically binds to a second tumor antigen.

24-25. (canceled)

26. The multispecific construct of claim Error!Reference source not found., wherein first antibody moiety is a Fab′ fused to N-terminus of the IgG Fc domain and the second antibody moiety is a sdAb fused to C-terminus of the IgG Fc domain, and the third antibody moiety is a scFv fused to N-terminus of the first antibody moiety.

27. (canceled)

28. The multispecific construct of claim Error!Reference source not found., wherein first antibody moiety is a Fab′ fused to N-terminus of a IgG Fc domain and the second antibody moiety is a sdAb fused to C-terminus of the IgG Fc domain, the third antibody moiety is a scFv fused to N-terminus of a pairing IgG Fc domain and the second antibody moiety is a sdAb fused to C-terminus of the pairing IgG Fc domain, wherein the pairing IgG Fc domain forms a heterodimer with the IgG Fc domain.

29. (canceled)

30. A pharmaceutical composition comprising the multispecific construct of claim 1, and a pharmaceutically acceptable carrier.

31. A nucleic acid encoding the multispecific construct of claim 1.

32-33. (canceled)

34. A method of treating a disease or condition in a subject in need thereof, comprising administering to the subject an effective amount of the multispecific construct of claim 1.

35. The method of claim 0, wherein the disease or condition is cancer.

36. (canceled)

37. An antibody or antigen-binding fragment thereof having specificity to a human Mucin 16 (MUC16) protein, wherein the antibody or antigen-binding fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein the HCDR1, HCDR2, HCDR3, LCDR1, LCDR2, and LCDR3 are selected from the group consisting of:

| | (a) |
|---|---|
| | HCDR1: |
| | (SEQ ID NO: 57) |
| | SYAIS, |
| | HCDR2: |
| | (SEQ ID NO: 58) |
| | GIIPIFGTANYAQKFQG, |
| | HCDR3: |
| | (SEQ ID NO: 59) |
| | DSRKYYYDSSGPALWGFDAFDI, |
| | LCDR1: |
| | (SEQ ID NO: 60) |
| | RASQSISSYLN, |
| | LCDR2: |
| | (SEQ ID NO: 61) |
| | AASSLQS, |
| | and |
| | LCDR3: |
| | (SEQ ID NQ: 62) |
| | QQSYSTLST, |
| | (b) |
| | HCDR1: |
| | (SEQ ID NO: 57) |
| | SYAIS, |
| | HCDR2: |
| | (SEQ ID NO: 58) |
| | GIIPIFGTANYAQKFQG, |
| | HCDR3: |
| | (SEQ ID NO: 67) |
| | EPPLSNYGDYATEQYYYGMDV, |
| | LCDR1: |
| | (SEQ ID NO: 60) |
| | RASQSISSYLN, |
| | LCDR2: |
| | (SEQ ID NO: 61) |
| | AASSLQS, |
| | and |
| | LCDR3: |
| | (SEQ ID NQ: 70) |
| | QQSYSTPLT, |
| | (c) |
| | HCDR1: |
| | (SEQ ID NO: 57) |
| | SYAIS, |
| | HCDR2: |
| | (SEQ ID NO: 58) |
| | GIIPIFGTANYAQKFQG, |
| | HCDR3: |
| | (SEQ ID NO: 75) |
| | APMVRGVPPTPYYYYYGMDV, |
| | LCDR1: |
| | (SEQ ID NO: 76) |
| | RASQSVSNYLA, |
| | LCDR2: |
| | (SEQ ID NO: 77) |
| | DASNRAT, |
| | and |
| | LCDR3: |
| | (SEQ ID NQ: 78) |
| | QQRSNWPS, |
| | (d) |
| | HCDR1: |
| | (SEQ ID NO: 57) |
| | SYAIS, |
| | HCDR2: |
| | (SEQ ID NO: 58) |
| | GIIPIFGTANYAQKFQG, |
| | HCDR3: |
| | (SEQ ID NO: 83) |
| | TPELLWFGELGGAYYFDY, |
| | LCDR1: |
| | (SEQ ID NQ: 84) |
| | RASESISSWLA, |
| | LCDR2: |
| | (SEQ ID NO: 85) |
| | KASTLEN, |
| | and |
| | LCDR3: |
| | (SEQ ID NO: 86) |
| | QQYRSHWSST, |
| | (e) |
| | HCDR1: |
| | (SEQ ID NO: 57) |
| | SYAIS, |
| | HCDR2: |
| | (SEQ ID NO: 58) |
| | GIIPIFGTANYAQKFQG, |
| | HCDR3: |
| | (SEQ ID NO: 91) |
| | ANFNIYYYYYGMDV, |
| | LCDR1: |
| | (SEQ ID NO: 92) |
| | RSSQSLLHSNGYNYLD, |
| | LCDR2: |
| | (SEQ ID NO: 93) |
| | LGSNRAS, |
| | and |
| | LCDR3: |
| | (SEQ ID NQ: 94) |
| | MQGTHWPRT, |
| | (f) |
| | HCDR1: |
| | (SEQ ID NO: 97) |
| | SYEMN, |
| | HCDR2: |
| | (SEQ ID NO: 98) |
| | RIKSKTDGGTTDYAAPV, |
| | HCDR3: |
| | (SEQ ID NO: 99) |
| | DLAAVAGLFDY, |
| | LCDR1: |
| | (SEQ ID NO: 100) |
| | QASQDISNYLN, |
| | LCDR2: |
| (SEQ ID NO: 101) | |
| | DASNLET, |
| | and |
| | LCDR3: |
| (SEQ ID NO: 102) | |
| | QQSYSTPWK, |
| | (g) |
| | HCDR1: |
| (SEQ ID NO: 57) | |
| | SYAIS, |
| | HCDR2: |
| (SEQ ID NO: 106) | |
| | RIIPIFGIANYAQKFQG, |
| | HCDR3: |
| (SEQ ID NO: 107) | |
| | TGDYDILTGSYYYGMDV, |
| | LCDR1: |
| (SEQ ID NQ: 108) | |
| | RASQGIRNDLG, |
| | LCDR2: |
| (SEQ ID NO: 61) | |
| | AASSLQS, |
| | and |
| | LCDR3: |
| (SEQ ID NO: 120) | |
| | LQDYNYPFT, |
| | (h) |
| | HCDR1: |
| (SEQ ID NO: 123) | |
| | DYYLS, |
| | HCDR2: |
| (SEQ ID NO: 58) | |
| | GIIPIFGTANYAQKFQG, |
| | HCDR3: |
| (SEQ ID NO: 125) | |
| | GGPHYDFWSGYTPGQHGGAFDI, |
| | LCDR1: |
| (SEQ ID NO: 126) | |
| | RASQSVSSSYLA, |
| | LCDR2: |
| (SEQ ID NO: 127) | |
| | GASSRAT, |
| | and |
| | LCDR3: |
| (SEQ ID NO: 128) | |
| | QQRSNWRNT, |
| | (i) |
| | HCDR1: |
| (SEQ ID NO: 57) | |
| | SYAIS, |
| | HCDR2: |
| (SEQ ID NO: 58) | |
| | GIIPIFGTANYAQKFQG, |
| | HCDR3: |
| (SEQ ID NO: 133) | |
| | DSGSSITMVRGGDYYYMDV, |
| | LCDR1: |
| (SEQ ID NO: 134) | |
| | RASQSVSSYLA, |
| | LCDR2: |
| (SEQ ID NO: 77) | |
| | DASNRAT, |
| | and |
| | LCDR3: |
| (SEQ ID NQ: 136) | |
| | QQRSNWPPT, |
| | (j) |
| | HCDR1: |
| (SEQ ID NO: 139) | |
| | YHAIS, |
| | HCDR2: |
| (SEQ ID NO: 140) | |
| | GIIPILGTANYAQKFQG, |
| | HCDR3: |
| (SEQ ID NO: 141) | |
| | GTTAARYYYYYYYMDV, |
| | LCDR1: |
| (SEQ ID NO: 100) | |
| | QASQDISNYLN, |
| | LCDR2: |
| (SEQ ID NO: 101) | |
| | DASNLET, |
| | and |
| | LCDR3: |
| (SEQ ID NO: 144) | |
| | QQYDNLPLT, |
| | (k) |
| | HCDR1: |
| (SEQ ID NO: 57) | |
| | SYAIS, |
| | HCDR2: |
| (SEQ ID NO: 58) | |
| | GIIPIFGTANYAQKFQG, |
| | HCDR3: |
| (SEQ ID NO: 149) | |
| | SITDYYDSSGYYFRPHENTGYYYGMDV, |
| | LCDR1: |
| (SEQ ID NO: 150) | |
| | RASQGINNYLA, |
| | LCDR2: |
| (SEQ ID NO: 151) | |
| | AASTLQS, |
| | and |
| | LCDR3: |
| (SEQ ID NO: 152) | |
| | QQYDTFSET, |
| | (l) |
| | HCDR1: |
| (SEQ ID NO: 57) | |
| | SYAIS, |
| | HCDR2: |
| (SEQ ID NO: 58) | |
| | GIIPIFGTANYAQKFQG, |
| | HCDR3: |
| (SEQ ID NO: 125) | |
| | GGPHYDFWSGYTPGQHGGAFDI, |
| | LCDR1: |
| (SEQ ID NO: 158) | |
| | RASQSISGWLA, |
| | LCDR2: |
| (SEQ ID NO: 159) | |
| | RTSYLES, |
| | and |
| | LCDR3: |
| (SEQ ID NO: 160) | |
| | QHYDTFSRA, |
| | or |
| | (m) |
| | HCDR1: |
| (SEQ ID NO: 139) | |
| | YHAIS, |
| | HCDR2: |
| (SEQ ID NO: 38) | |
| | SISSGGNTYYPDTVKGR, |
| | HCDR3: |
| (SEQ ID NO: 165) | |
| | EGPDYGDYSWSMDYYYGMDV, |
| | LCDR1: |
| (SEQ ID NO: 166) | |
| | RASQSVNSRYLA, |
| | LCDR2: |
| (SEQ ID NO: 167) | |
| | GASTRAT, |
| | and |
| | LCDR3: |
| (SEQ ID NO: 168) | |
| | QQYGTFSIT. |

38-39. (canceled)

40. The antibody or antigen-binding fragment thereof of claim 37, comprising
(a) a heavy chain variable region comprising the amino acid sequence of SEQ ID NO: 63, and a light chain variable region comprising the amino acid sequence of SEQ ID NO: 64;
(b) a heavy chain variable region comprising the amino acid sequence of SEQ ID NO: 71, and a light chain variable region comprising the amino acid sequence of SEQ ID NO: 72;
(c) a heavy chain variable region comprising the amino acid sequence of SEQ ID NO: 79, and a light chain variable region comprising the amino acid sequence of SEQ ID NO: 80;
(d) a heavy chain variable region comprising the amino acid sequence of SEQ ID NO: 87, and a light chain variable region comprising the amino acid sequence of SEQ ID NO: 88;
(e) a heavy chain variable region comprising the amino acid sequence of SEQ ID NO: 95, and a light chain variable region comprising the amino acid sequence of SEQ ID NO: 96;
(f) a heavy chain variable region comprising the amino acid sequence of SEQ ID NO: 103, and a light chain variable region comprising the amino acid sequence of SEQ ID NO: 104;
(g) a heavy chain variable region comprising the amino acid sequence of SEQ ID NO: 121, and a light chain variable region comprising the amino acid sequence of SEQ ID NO: 122;
(h) a heavy chain variable region comprising the amino acid sequence of SEQ ID NO: 129, and a light chain variable region comprising the amino acid sequence of SEQ ID NO: 130;
(i) a heavy chain variable region comprising the amino acid sequence of SEQ ID NO: 137, and a light chain variable region comprising the amino acid sequence of SEQ ID NO: 138;
(j) a heavy chain variable region comprising the amino acid sequence of SEQ ID NO: 145, and a light chain variable region comprising the amino acid sequence of SEQ ID NO: 146;
(k) a heavy chain variable region comprising the amino acid sequence of SEQ ID NO: 153, and a light chain variable region comprising the amino acid sequence of SEQ ID NO: 154;
(l) a heavy chain variable region comprising the amino acid sequence of SEQ ID NO: 161, and a light chain variable region comprising the amino acid sequence of SEQ ID NO: 162; or
(m) a heavy chain variable region comprising the amino acid sequence of SEQ ID NO: 169, and a light chain variable region comprising the amino acid sequence of SEQ ID NO: 170.

41-42. (canceled)

43. A bifunctional molecule, comprising a first antibody moiety having specificity to a human MUC16 protein and a second antibody moiety having specificity to a second protein, wherein the first antigen-binding moiety comprises an antibody or antigen-binding fragment thereof of claim 37.

44-48. (canceled)

49. The bifunctional molecule of claim 43, wherein the second antibody moiety comprises HCDR1 of SNCMG (SEQ ID NO: 24), HCDR2 of VICTGGGSPSYADSVKG (SEQ ID NO: 25), and HCDR3 of DLLRAGTPLSSYEFNY (SEQ ID NO: 26).

50-55. (canceled)
